(19) United States
(12) Patent Application Publication
(10) Pub. No.: US 2004/0063093 A1
(43) Pub. Date: Apr. 1, 2004

(54) Recombinant feline coronavirus S proteins

(75) Inventors: Timothy J. Miller, Malvern, PA (US); Albert Paul Reed, Exton, PA (US); Sharon R. Klepfer, Clifton Heights, PA (US); Nancy E. Pfeiffer, Seward, NE (US); Brian T. Suiter, Lincoln, NE (US); Elaine V. Jones, Wynnewood, PA (US)

Correspondence Address: SCULLY SCOTT MURPHY & PRESSER, PC, 400 GARDEN CITY PLAZA, GARDEN CITY, NY, 11530

(73) Assignee: Pfizer, Inc., New York, NY

(21) Appl. No.: 10/646,874

(22) Filed: Aug. 22, 2003

Related U.S. Application Data
(63) Continuation of application No. 09/854,799, filed on May 14, 2001, now Pat. No. 6,642,359.
Application No. 09/854,799 is a continuation of application No. 08/392,459, filed on Feb. 22, 1995, now Pat. No. 6,280,974.
Application No. 08/392,459 is a continuation of application No. 07/847,018, filed on Apr. 8, 1992, now abandoned.

(51) Int. Cl. C12Q 1/70; C07K 14/165

(52) U.S. Cl. 435/5; 530/350

(57) ABSTRACT

The present invention relates to polypeptides and proteins useful in the diagnosis and prevention of disease caused by feline infectious peritonitis virus (FIPV) and feline enteric coronavirus (FECV).

### FIELD OF THE INVENTION

This invention relates generally to polypeptides useful for diagnosis and both preventive and prophylactic treatment of feline infectious peritonitis virus disease. More specifically, the invention relates to new recombinant feline coronavirus S proteins and fusion proteins.

### BACKGROUND OF THE INVENTION

Feline Infectious Peritonitis (FIP) is a highly lethal disease in both wild and domestic cats, occurring predominantly in young animals although cats of all ages are susceptible. Symptoms of FIP may include anemia, neutrophilia, increased concentrations of immunoglobulin and/or fibrinogen, renal damage as indicated by high levels of urea and creatinine, and disseminated intravascular coagulation.

Previous attempts to develop an effective FIPV vaccine have been largely unsuccessful. Administration of traditional inactivated whole virus vaccines have actually predisposed cats to the development of FIP and produced a more rapid and fulminating disease after challenge. Cats vaccinated with an avirulent strain of FIPV were more readily infected than non-immunized cats and animals immunized with a sublethal dose of virulent FIPV showed inconsistent protection from challenge [Pedersen and Black, Am. J. Vet. Res., 44:229-234 (1983)].

Immunization of cats with other antigenically related coronaviruses has also not been successful. In most experiments, the administration of TGEV, CCV and human coronavirus 229E has neither sensitized nor protected cats [Woods and Pedersen, Vet. Microbiol., 4:11-16 (1979); Toma et al, Rec. Med. Vet., 155:788-803 (1979); Barlough et al, Can. J. Come. Med., 49:303-307 (1985); Barlough et al, Lab. Anim. Sci., 34:592-597 (1984); Stoddart et al, Res. Vet. Sci., 45:383-388 (1988)].

Recently, a temperature-sensitive FIPV (TS-FIPV) vaccine has been developed which, when administered intranasally, is efficacious and safe upon FIPV challenge [Christianson et al, Arch. Virol., 109:185-196 (1989)]. This vaccine has-limited efficacy when administered subcutaneously, but appears to be effective against homologous and heterologous strains. Generally, intranasal administration is not preferred because the dosage amount is less quantifiable than other routes.

There remains a need for effective diagnostic, therapeutic and protective compositions for use in diagnosing, treating, and vaccinating animals against FIPV and serologically related infections.

### SUMMARY OF THE INVENTION

In one aspect, the invention provides protein and peptide fragments of a feline coronavirus S gene. These peptides may be expressed recombinantly or synthetically and are useful as diagnostic, therapeutic or vaccinal components. In one embodiment, the feline coronavirus S-derived peptides fall within the range of amino acid numbers 1 to about 1454 of the S genomes of a variety of FIPV strains and 1 to about 1454 of the FECV S genome, or smaller peptide fragments therein. In a preferred embodiment, the feline coronavirus S-derived peptides fall within the range of amino acid numbers 1 to about 748 of the S genes of the FIPV strains or 1 to about 748 of the FECV S genome (SEQ ID NO: 32]. More particularly, peptides falling within the range of about amino acid #94 to about amino acid #223 of the FIPV or FECV S genomes are desirable. In a particularly preferred embodiment, the feline coronavirus S-derived peptides are found to be within the range of amino acid #97-222 of the FIPV or FECV S genomes. In still another embodiment, peptides falling within the range of about amino acids #121 to about amino acid #180 of the FIPV or FECV genome are disclosed.

Peptide fragments of the invention are capable of distinguishing between FIPV and FECV, or different strains of FIPV when used in diagnostic assays, such as enzyme linked immunosorbant assays (ELISA) or Western Blots. These peptides may also be used as antigens to screen cat sera for the presence of antibody or to generate antibodies capable of distinguishing between FIPV and FECV, or different strains of FIPV.

In another aspect, the present invention provides nucleotide sequences from FIPV and FECV within the regions of nucleotide #1 to about #4365 and #1 to about #2246, which encode the above-described peptides, or which flank the above-described peptide-encoding sequences. These nucleotide sequences are capable of distinguishing between the FIPV and FECV S genomes, when they are used in diagnostic assays as PCR primers or hybridization probes.

Another aspect of the invention provides novel recombinant FIPV or FECV S fusion proteins. The feline coronavirus S-derived peptides of the present invention may be fused with a selected protein which confers a desired advantage upon recombinant expression of the S peptide. For example, the fusion partner may be a protein which is highly expressed in the desired host cell system or which is characterized by a high degree of secretion. The fusion partner may also be a signal sequence or a sequence which enhances the stability of the S-derived peptide in a selected host cell system. In one embodiment of this aspect, peptides derived from the S gene of feline coronavirus are fused with the N-terminal 52 amino acids of galactokinase (GalK).

In another aspect the present invention provides a diagnostic reagent composition which comprises an FIPV S-derived peptide or fusion protein of the present invention, optionally associated with a detectable label. Such diagnostic reagents may be used to assay for the presence of FIPV or FECV in cats using standard assay formats.

In a similar aspect the present invention provides a diagnostic reagent composition which comprises a nucleotide sequence encoding or flanking an FIPV S-derived peptide or fusion protein of the present invention, the DNA sequence being optionally associated with a detectable label. Such diagnostic reagents may be used to assay for the presence of FIPV or FECV in cats in hybridization assays or in the PCR technique.

In still another aspect of the present invention, the S-derived peptides and/or the S-derived fusion proteins may be utilized as the active component in vaccines to protect animals against infection with FIPV or FECV. A vaccine composition includes an effective amount of an FIPV or FECV S-derived peptide or fusion protein of the present invention capable of stimulating immunity against one or more virulent feline coronaviruses and a carrier suitable for internal administration. Additionally, characterization of the immune response to these peptides and proteins may also suggest other region(s) of the FIPV or FECV sequences which should be included in vaccines.

In yet a further aspect, the present invention provides a pharmaceutical composition for the treatment of FIPV or FECV infection comprising a therapeutically effective amount of a FIPV or FECV S-derived peptide or fusion protein of the invention and a pharmaceutically effective carrier.

In still another aspect, the invention provides a diagnostic kit which may be used by veterinarians to identify cats which are uninfected or which have been exposed to FECV or native FIPV. The kit will also allow the identification of cats which have been vaccinated against these diseases. Such a kit may also allow one to distinguish between different strains of FIPV, or to identify cats at advanced stages of FIPV infection. The kit may be comprised of PCR primers of this invention selected from the S gene nucleotide sequences; a selected FIPV S-derived peptide or fusion protein; primers, peptides and fusion proteins of related or similar viruses, and primers, peptides and fusion protein-encoding regions from a “consensus” sequence as described below.

In a further aspect, the invention provides a method of using the PCR S-derived primers and/or the S-derived peptides and fusion proteins of this invention to identify previously exposed and naive cats, as well as to differentiate exposure to FIPV from exposure to other related coronaviruses. Another diagnostic method of this invention permits the use of an S-gene derived peptide in an ELISA to detect an antibody to the virus in cat sera.

Another aspect of this invention involves a method of vaccinating an animal against infection with FIPV by administering an effective vaccinal amount of an S-derived peptide or an S-derived fusion protein of this invention.

In still a further aspect, the invention provides a method for treating FIPV infection by administering to an animal a pharmaceutical composition of the present invention.

Still another aspect of this invention is an antibody directed to FIPV or FECV or related coronavirus epitopes, which antibody is capable of distinguishing between these viruses. These antibodies are generated by employing a peptide or fusion protein of the present invention as an antigen. Such antibodies may also be employed as diagnostic or therapeutic reagents, and may be optionally attached to a detectable label or toxin or other therapeutic compound.

Other aspects and advantages of the present invention are described further in the following detailed description of embodiments of the present invention.

### BRIEF DESCRIPTION OF THE DRAWINGS

FIG. 1 is a schematic diagram of the pOTSKF33 bacterial expression vector.

FIG. 2 illustrates a plasmid containing a PCR-amplified fragment cloned into the XmaI-StuI sites of pOTSKF33.

FIG. 3 illustrates the nucleotide [SEQ ID NO: 19] and amino acid sequence [SEQ ID NO: 20] of the PCR expression clone AR58-3.

FIG. 4 illustrates the S gene nucleotide and amino acid sequences of DF2 FIPV [SEQ ID NO: 21 and 22]. Also illustrated is a fragment of the sequences of DF2-HP [SEQ ID NO: 23 and 24] which are identical to the sequences of DF2 FIPV (to the extent DF2 FIPV has been sequenced) with the exception of the nucleotide changes above and amino acid differences below the DF2-HP sequences.

FIG. 5 illustrates a fragment of the S gene TS-BP nucleotide sequence [SEQ ID NO: 27] and amino acid sequence [SEQ ID NO: 28] by indicating the positions where the sequences differ from the sequences of TS FIPV [SEQ ID NO: 25 and 26]. The entire TS FIPV S gene sequence is provided.

FIG. 6 illustrates a fragment of the S gene nucleotide and amino acid sequences [SEQ ID NO: 29 and 30] of TN406.

FIG. 7 illustrates the complete nucleotide and amino acid sequences [SEQ ID NO: 31 and 32] of FECV S gene.

FIG. 8 illustrates fragments of the nucleotide and amino acid sequences of the UCD-2 S gene.

FIG. 9 illustrates the nucleotide and amino acid sequences [SEQ ID NO: 33 and 34] of a consensus partial S gene sequence.

### DETAILED DESCRIPTION OF THE INVENTION

The present invention provides novel compositions useful for FIPV and FECV diagnostic, vaccinal and therapeutic compositions as well as methods for using these compositions in the diagnosis, prophylaxis and treatment of FIP.

It is presently preferred to use the FIPV or FECV S gene or a portion thereof to construct the peptides useful in this invention. However, the S gene from other coronaviruses may be useful in a similar manner as that disclosed in this invention.

The S gene sequence from the published FIPV WT WSU 1146 strain was analyzed by computer analysis, as described in detail in Example 1, resulting in the prediction of antigenic regions which may differentiate virus strains. The inventors predicted that differences between various strains of FIPV and its sister virus, FECV, would be localized within the amino terminal half of the peplomer sequence. Using discrete portions of the S protein that differ in amino acid sequence, polypeptides could be used to generate reagents that discriminate between the serologically similar viruses.

The examples below specifically refer to the published FIPV strain WT WSU 1146 [DeGroot et al, J. Gen. Virol., 68:2639-2646 (1987)], and to newly identified sequences from strains WT DF2, TS-FIPV, WT TN406, WT UCD-1, and WT UCD-2 and to vaccine strains WT FIPV DF2 high passage (DF2-HP) and TS FIPV DF2 back passage (DF2-BP). WT FIPV DF2-HP was derived from WT DF2 by 99 serial passes in tissue culture. The DF2-HP was then mutagenized by exposure to ultraviolet light to generate the TS FIPV virus. To determine the stability of the TS FIPV virus, it was then passaged 5 times in cats and tissue culture to generate the TS-BP FIPV strain. Particularly disclosed are the complete nucleotide and amino acid sequences of the FECV S gene. DNA and amino acid sequences of a putative consensus sequence are also, useful in providing nucleotide and peptide sequences of this invention. The present invention is not limited to the particular FIPV strains employed in the examples. According to the teachings of this invention, the same analysis may be made from other virulent or avirulent feline or other coronavirus strains with similar results.

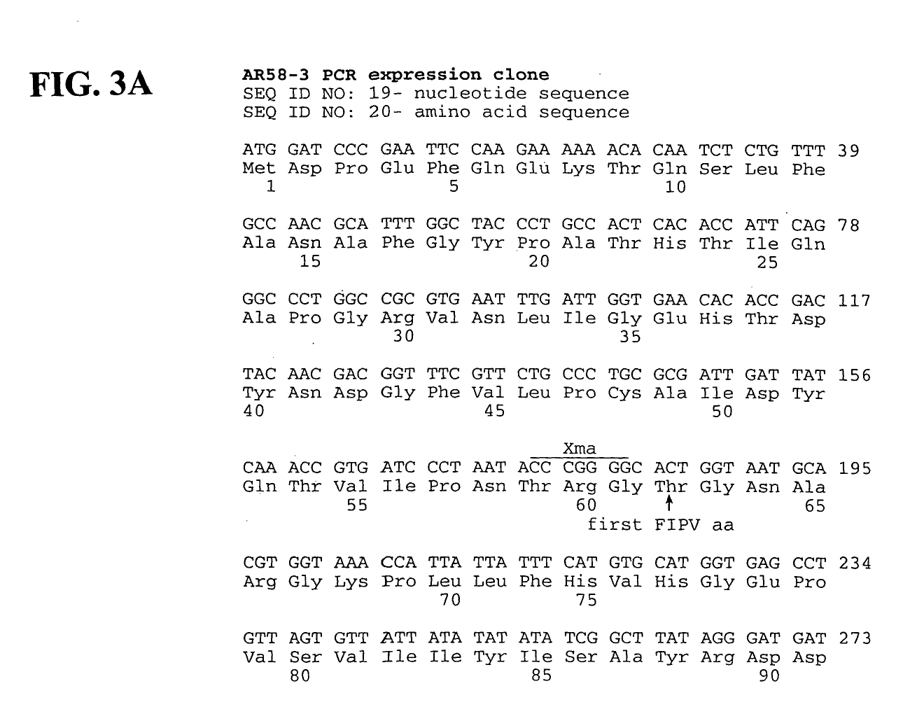

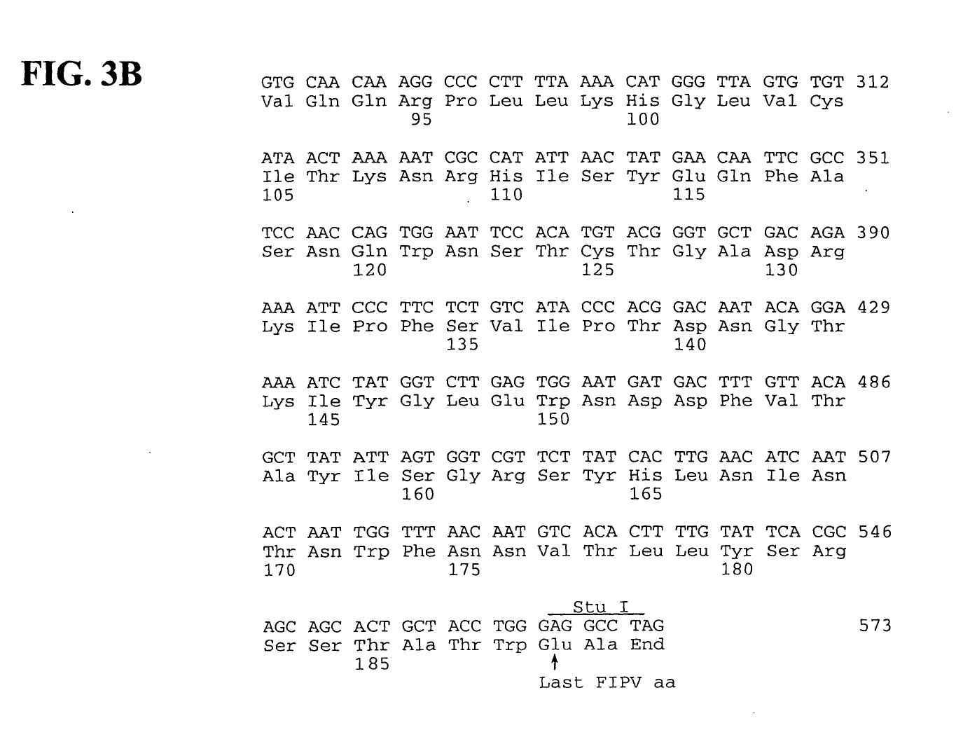

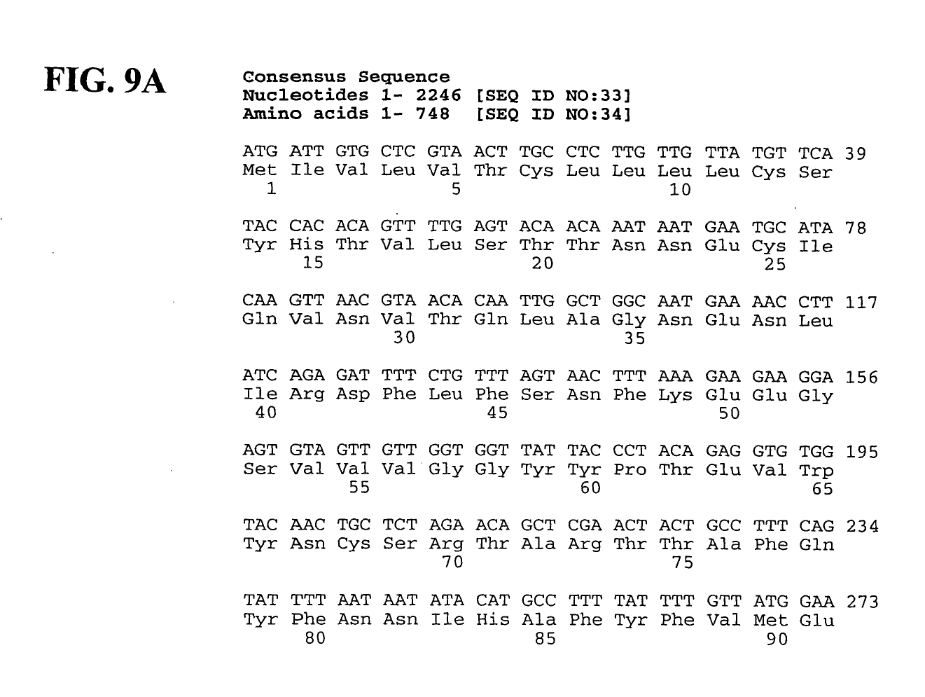

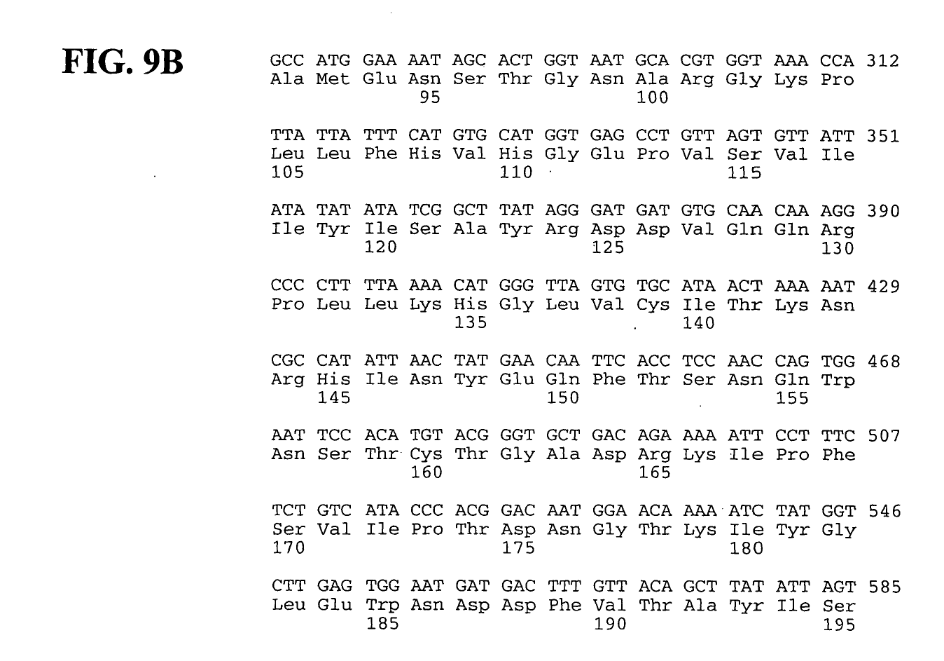

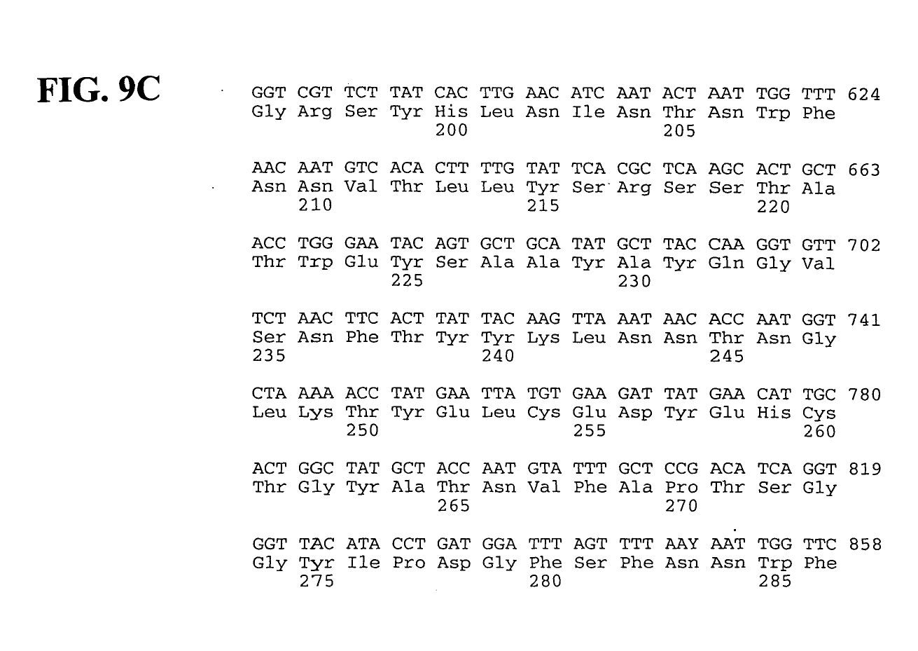

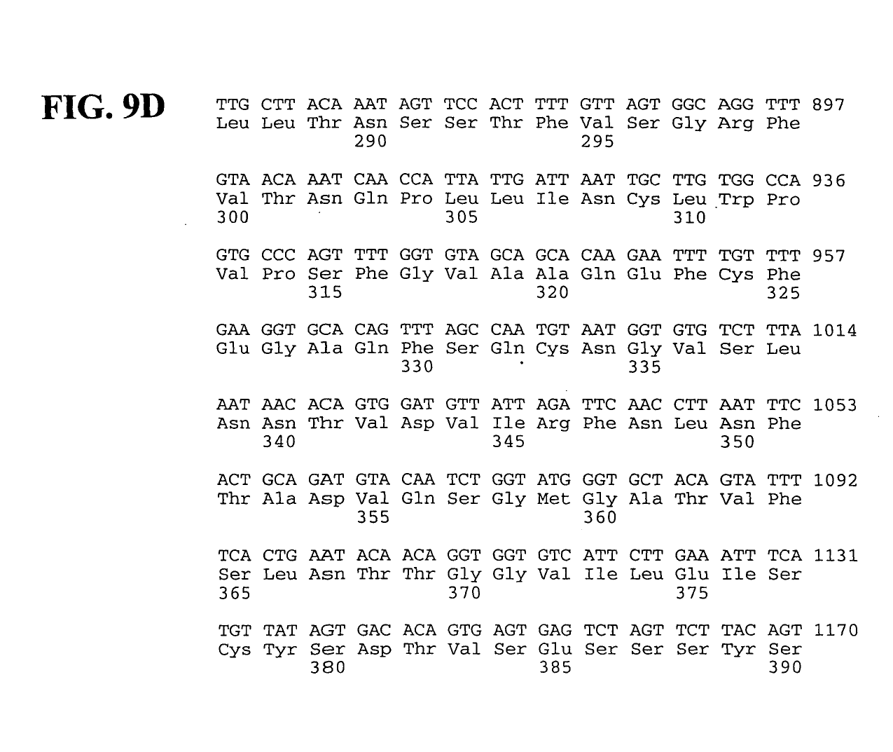

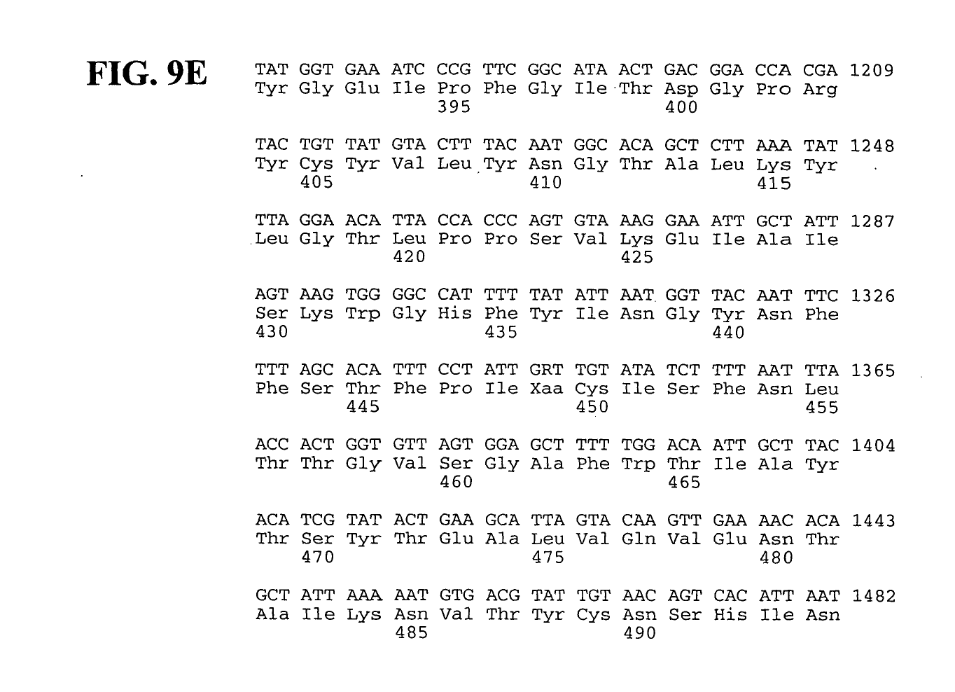

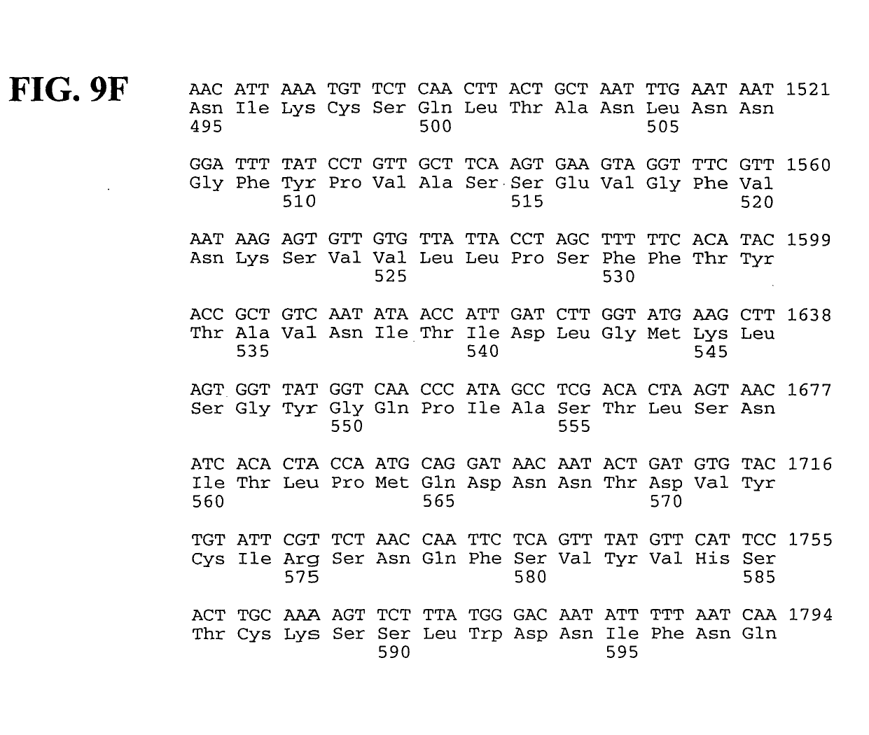

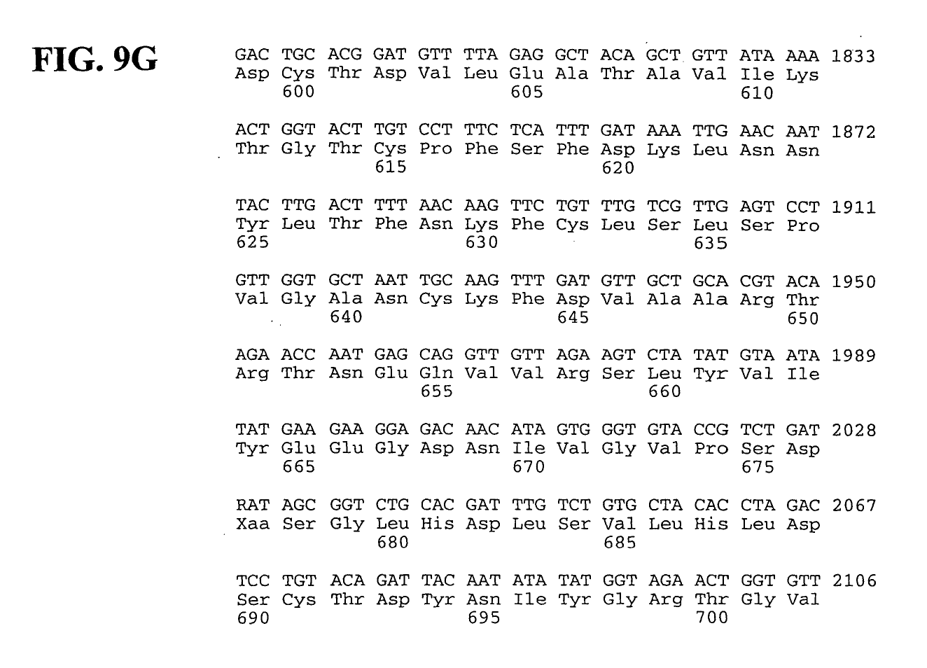

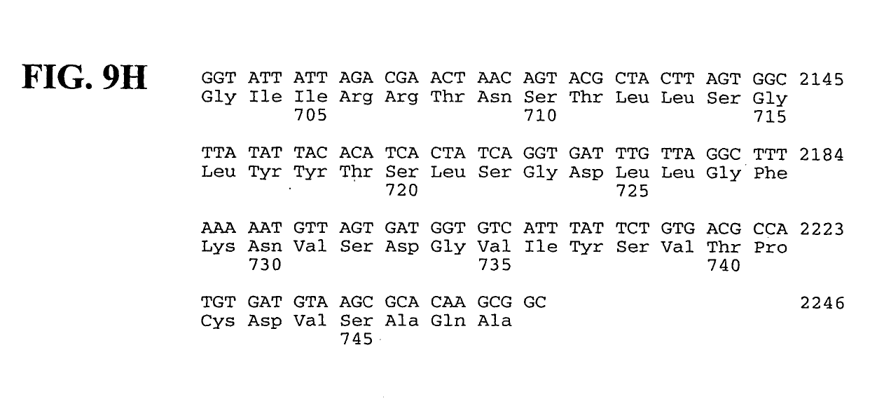

The amino acid and nucleotide numbers of the S-derived peptides and DNA sequences described herein from unpublished or newly identified FIPV or FIPV-related virus strains correspond to the numbering system of the published WT WSU 1146 S gene. However, as indicated in the viral sequences appearing in FIGS. 3-8 and by the formation of the consensus sequence of FIG. 9 and as described in detail in Example 12, the sequences in the other viruses are somewhat longer or shorter than the identified homologous WT WSU 1146 peptides, and the actual amino acid numbering of homologous WT WSU 1146 sequence regions in these previously unknown virus sequences differ. The consensus sequence of FIG. 9 is an artificial sequence which includes the most commonly employed amino acid in each position among the FIPV sequences WT WSU 1146, WT DF2, DF2-HP, TS, TS-BP, WT TN-406, and FECV.

The DNA and protein sequences from which regions suitable as candidates for differentiating between FIPV strains and FECV have been identified and are present in the variable N-terminal half of the S gene of both the FIPV strains, the consensus sequence, and FECV. DNA and protein sequences from the carboxy half of the S gene are also identified as possible vaccinal components. All of these regions may be cloned and expressed by conventional means. The location of polymerase chain reaction (PCR) primers can be shifted to amplify sequences spanning the entire S gene, and/or discrete portions of the gene.

In the practice of this invention, oligonucleotide sequences were designed to prime cDNA synthesis at specific sites within the FIPV S gene. Oligonucleotide primers specific for the DNA sequence of the FIPV S gene were designed as described in detail in Example 2. Table II below specifically identifies the 5′ and 3′ FIPV S oligonucleotide primers [SEQ ID NOS: 1-9 and 10-18, respectively] by nucleotide sequence and portion of S gene amino acid sequence covered. In addition to providing nucleotide sequences spanning the amino acid sequence regions of the S gene, the primers specifically identified in Table II [SEQ ID NOS: 1-18] also contain sequences for introducing a feline coronavirus S gene fragment in a specific orientation into a selected expression vector to produce fusion proteins of the invention.

These same primers, as well as the below-described optimized conditions for the PCR amplification of fragments from feline coronavirus RNA, e.g., the primers of Table II below [SEQ ID NOS: 1-18], may also be utilized as reagents in a diagnostic method employing the PCR technique to identify the presence of an FIPV or FIPV-like virus.

These primers were synthesized by the phosphoramidite method and gel purified prior to use. The primers were then used in the technique of polymerase chain reaction (PCR) analysis [see, e.g., Arnheim et al, Chem. & Eng. News, pages 36-47 (Oct. 1, 1990)], which reference is incorporated herein by reference. The PCR technique is known to those of skill in the art of genetic engineering and is described in detail in Example 3. The PCR technique may be used to generate additional fragments representing discrete regions of the FIPV and FECV peplomer gene. Thus this technique permits the isolation, identification and amplification of FIPV and FECV sequences which represent areas of homology or heterogeneity among significant strains of feline coronaviruses. Such DNA sequences or fragments thereof are useful in both diagnosis and therapy of infected animals.

The identification of heterogenous gene sequences provides reagents useful in diagnostic assays to detect and distinguish the presence of specific viruses from each other, e.g., to distinguish one feline coronavirus from another or one species of coronavirus from another by means of conventional assay formats.

PCR analysis of related feline coronaviruses also generates information on regions of homology or non-homology among virus strains with different disease-causing characteristics. Information obtained by the PCR mapping of the feline coronavirus and other related viruses, such as porcine transmissible gastroenteritis virus (TGEV) [Jacobs et al, Virus Res., 8:363-371 (1987)] canine CCV and human 229E, is useful in formulating vaccines effective against other closely related coronaviruses or to more than one FIPV strain. For example, exemplary vaccines may contain effective amounts of the above-described homologous amplified sequences, possibly effective against more than one species of coronavirus.

Briefly described, PCR employs two oligonucleotide primers which are complementary to the opposite strands of a double stranded nucleic acid of interest which strands are oriented such that when they are extended by DNA polymerase, synthesis occurs across the region which separates the oligonucleotides. By repeated cycles of heat denaturation, annealing of the primers to their complementary sequences and extension of the annealed primers with a temperature stable DNA polymerase, millions of copies of the target gene sequence are generated.

The template for the reaction is total RNA, which is isolated from FIPV infected cells. DNA fragments generated by PCR were amplified from cDNA which had been synthesized from this RNA. In initial experiments, the RNA was purified and prepared from the following-strains of FIPV or FIPV-related viruses: WT FIPV DF2, WT FIPV WSU 1146, TS FIPV DF2, WT FIPV UCD-2, WT FIPV TN406, FECV and WT FIPV UCD-1. The RNA and cDNA preparation is described in detail in Example. 3 below. Other strains of FIPV or FIPV-related sequences may also provide PCR templates in a similar manner.

The specific regions of the S gene which are amplified by PCR permit differentiation of the feline coronavirus and other related viruses. Mixing and matching the oligonucleotide primers permitted the synthesis of regions representing as little as 105 amino acids of S or as large as 1454 amino acids (complete S). Such primers are identified in Table II below. As described in Example 4 below, PCR primers designed to span amino acid #94-223, produced the following amplified fragments of the FIPV S gene among which are shorter peptides than the spanned region. Presently preferred peptides are those spanning from about amino acid number 94 to about amino acid number 223 of the FIPV S genome the consensus sequence and the FECV genome, and more particularly, from about amino acid number 97 to about amino acid number 222 of the FIPV S genome, the consensus sequence and the FECV genome.

Specific amplified sequences of the FIPV strains, of the invention and FECV include the regions recited below:

From WT DF2, the amplified regions spanned amino acids #1-105, 1-223, 1-362, 1-555, 1-748, 1-1040, 1-1203, 1-1452, 94-223, 94-362, 94-555, 94-748, 94-1040, 94-1203, 94-1452, 213-362, 213-555, 213-748, 213-1040, 213-1203, 213-1452, 352-555, 352-748, 544-748, 544-905, 544-1040, 554-1203, 554-1452, 737-905, 737-1040, 737-1203, 737-1452, 894-1040, 894-1203, 894-1452, 1029-1203, 1029-1452, and 1192-1452.

From TS DF2, the amplified regions spanned amino acids #1-105, 1-223, 1-362, 1-555, 1-748, 1-1040, 1-1203, 94-223, 94-362, 94-555, 94-748, 94-1040, 94-1203, 94-1452, 213-362, 213-555, 213-748, 213-1040, 213-1203, 213-1452, 352-748, 544-748, 544-905, 544-1040, 544-1203, 544-1452, 737-905, 737-1040, 737-1203, 737-1452, 894-1040, 894-1203, 894-1452, 1029-1203, 1029-1452, and 1192-1452.

From FECV, the amplified regions spanned amino acids #1-105, 1-223, 1-362, 94-223, 94-362, 94-555, 94-748, 94-1040, 213-362, 213-748, 352-555, 352-748, 544-748, 544-905, 544-1040, 544-1203, 544-1452, 737-905, 737-1040, 737-1203, 737-1452, 894-1040, 894-1203, 894-1452, 1029-1203, 1029-1452, and 1192-1452.

From WT WSU 1146, the amplified regions spanned amino acids #1-105, 1-223, 1-362, 1-555, 94-223, 94-362, 94-555, 94-748, 213-362, 213-748, 352-555, 352-748, 544-748, 544-905, 544-1040, 544-1203, 737-905, 737-1040, 737-1203, 894-1040, 894-1203, 894-1452, 1029-1203, 1029-1452, and 1192-1452.

From WT UCD-1, the amplified regions spanned amino acids #94-223, 94-362, 352-555, 352-748, 544-748, 737-905, 737-1040, 737-1203, :894-1040, 894-1203, 1029-1203, 1029-1452, and 1192-1452.

From WT TN406, the amplified region spanned amino acids #94-223. From WT UCD-4, the amplified region spanned amino acids #94-223.

Many of these fragments have been cloned and expressed as galK fusion proteins. They are listed in Table IV of Example 5 below.

Similarly, PCR DNA fragments were isolated which show areas of homology or heterogeneity among different strains. For example, the DNA primers flanking amino acid #737-1452 of the FIPV or FECV S genomes provide fragments of predicted size (2168 bp) and DNA primers flanking amino acid #1029-1452 of the FIPV and FECV sequences provide fragments of predicted size (1290 bp). These fragments were amplified from each of the DF2, TS and FECV viral templates. DNA fragments spanning amino acids #1-748 were amplified from DF2, DF2-HP, TS-BP, TS and FECV. A DNA fragment was also amplified for amino acids #94-223 for WT TN406.

Specific fragments which were not amplified, despite appropriate priming events, included the fragments extending from amino acid #1-555 and 352-555 for FECV, indicating regions of suspected heterogeneity with the WSU 1146 based primers. These polypeptides or shorter fragments thereof are useful in distinguishing FECV from the FIPV strains.

After identifying roughly homologous regions of the S gene sequence and of the amino acid sequences encoded thereby, the sequences were compared to determine their percent homologies. In general, nucleic acid and amino acid homologies of less than 95% may indicate that certain regions of the virus may be useful as a diagnostic capable of distinguishing between the apathogenic FECV and the virulent FIPV. The following Table I illustrates the homologies between the S gene regions of the FIPV strains indicated and FECV, indicating the FECV and the FIP viruses were sufficiently different to supply useful differentiating sequences for diagnostic and therapeutic use.

Homologies reported in the Table I are in percent and numbers of mismatching/nonmatching base pairs or amino acids are in parentheses. AA (I) represents perfect match amino acid homology. AA (S) represents similarity match amino acid homology based on the rules of M. O. Dayhoff, “Sequence and Atlas of Protein Structure”, National Biomedical Research Foundation, Silver Spring, Md. (1968).

TABLE I

| Strain 1 | Strain 2 | Nucleic Acid | AA (I) | AA (S) |
|---|---|---|---|---|
| WSU | FECV | 92.9 (159) | 93.0 (52) | 93.0 (52) |
| DF2 | FECV | 93.1 (154) | 93.3 (50) | 93.3 (50) |
| DF2-HP | FECV | 93.0 (158) | 93.3 (50) | 93.3 (50) |
| TS | FECV | 92.9 (160) | 92.9 (53) | 92.9 (53) |
| TS-BP | FECV | 93.1 (156) | 93.3 (50) | 93.3 (50) |
| TN406 | FECV | 90.0 (37) | 86.1 (17) | 86.1 (17) |

Comparison of the nucleotide and amino acid sequences of the six FIP coronaviruses WT DF2, WT WSU 1146, DF2-HP, TS, TS-BP, and 14T TN406 to FECV and to the Consensus Sequence (FIG. 9) revealed that overall, FECV shares only ˜93.0% homology with the FIPV strains. Greater than 50 amino acids differ between FECV and the illustrated FIPV strains in the first 748 amino acids of the S gene. Some of these changes occur in clusters in regions of the FECV sequence which differ from homologous regions of the FIPV sequences. Such clustered regions represent sites for differentiation of the virus and are desirable as diagnostic reagents capable of distinguishing between FIPV and FECV or as therapeutic or vaccinal agents. Corresponding regions of the FIPV strains or consensus sequence, i.e., regions demonstrating clustered amino acid differences from FECV or other strains of FIPV, may be employed in the same way.

The nucleotide sequence of the S gene of FECV provides desirable sequences for hybridization probes and PCR primers, e.g., the sequence between base pairs 1-1080. Corresponding amino acid sequences provide peptides useful in ELISA or Western assay or as antigens for the screening of sera or development of antibodies, e.g., the sequence between amino acids 1-360. Such probes, primers, antigens and antibodies would react positively with tissue or serum samples of cats infected with FECV, but negatively with cats infected with a FIPV strain.

In particular, the following regions of FECV appear particularly suitable for the generation of peptide fragments and DNA sequences for such purposes. Corresponding regions of the FIPV strains and consensus sequence may also be useful for the same purposes.

These FECV regions are: amino acid residues 18-26 [SEQ ID NO: 36], 46-53 [SEQ ID NO: 38], 73-78 [SEQ ID NO: 40], 124-174 [SEQ ID NO: 42], 145-150 [SEQ ID NO: 44], 138-159 [SEQ ID NO: 46], 143-150 [SEQ ID NO: 48], 200-205 [SEQ ID NO: 50], and 529-536 [SEQ ID NO: 52] and corresponding nucleotide fragments 52-78 [SEQ ID NO: 35], 136-159 [SEQ ID NO: 37], 214-231 [SEQ ID NO: 39], 370-519 [SEQ ID NO: 41], 433-450 [SEQ ID NO: 43], 412-477 [SEQ ID NO: 45], 427-450 [SEQ ID NO: 47], 598-615 [SEQ ID NO: 49], and 1585-1608 [SEQ ID NO: 51].

Smaller peptide fragments in these regions or larger fragments containing these regions may be employed in biological and serological assays, e.g. at least 10 amino acids in length. Preferably, a sequence of at least 7 or 8 different amino acids in a peptide of 15 amino acids is needed for most conventional veterinarian performed assays [see, Posthumus et al, J. Virol., 68:2639-2646 (1987)]. Of course, genetic techniques are capable of detecting a single amino acid change in a small peptide.

Smaller or larger DNA fragments in these regions may also be employed as PCR primers or hybridization probes. Desirably PCR primer sequences are between 15 to 30 bases in length, with an intervening sequence of at least 100 bases to as large as 1500 bases there between, according to conventional PCR technology. However, it is possible that larger or smaller sequence lengths may be useful based upon modifications to the PCR technology.

In general, in order to achieve satisfactory discrimination, a probe made up of one or more of these sequences would consist of between 15 and 50 bases in length based on current technology. However, shorter regions may be used if they are bound to a carrier. Suitable carriers include ovalbumin, keyhole limpet hemocyanin, bovine serum albumin, sepharose beads and polydextran beads.

The PCR amplification technique itself may be used as a diagnostic tool. Using protocols similar to those used for forensic purposes, tissue or blood samples from a cat suspected to be infected with FIPV would be subjected to PCR amplification with a selected FIPV-specific set of primers, such as those DNA sequences disclosed above and in Table II. Amplification of DNA would correlate to the presence of FIPV. Absence of FIPV in the sample would result in no amplification. Similarly, the selection of specific sets of S primers would allow the identification of a particular strain of FIPV as well. Similar results may be obtained to diagnose FECV using FECV primers to other regions of heterogeneity vs. FIPV strains, as indicated above.

When used as diagnostic reagents, the primers, probes and peptides of this invention may be optionally associated with detectable labels or label systems known to those skilled in the art. The diagnostic assays may be any conventionally employed assay, e.g., a sandwich ELISA assay, a Western blot, a Southern blot and the like.

It is anticipated that PCR primers, hybridization probes and, alternatively peptide diagnostic reagents, could be similarly designed to distinguish CCV and TGEV from FIPV. For example, the PCR amplification of nucleic acid from a sample tissue or biological fluid from an animal suspected of infection using primers specific for regions of viral gene sequences may identify or rule out the presence of a specific virus. Thus, appropriate treatments may be selected for the infected animal.

The nucleotide and peptide fragments of the S genes of feline coronaviruses according to this invention may be readily synthesized by conventional means, e.g., Merrifield synthesis [Merrifield, J.A.C.S, 85:2149-2154 (1963)]. Alternatively, they may be produced by recombinant methods. Cloning procedures are conventional and as described by T. Maniatis et al, Molecular Cloning (A Laboratory Manual), Cold Spring Harbor Laboratory (1982).

A selected PCR-derived fragment of-this invention representing a portion of the S gene sequence, as determined by a fragment produced during PCR, is cloned into a selected expression vector. Vectors for use in the method of producing S gene proteins comprise a novel S gene fragment DNA sequence of the invention and selected regulatory sequences in operative association with the DNA coding sequence, capable of directing the replication and expression of the S-derived peptide in a selected host cell.

The above-identified S gene nucleotide sequences, proteins and peptide fragments are also desirably produced in the form of fusion proteins. Such fusion proteins may be produced synthetically as described above for the peptide fragments themselves. However, to facilitate the production of fusion proteins of this invention, recombinant methods are preferred. The selected primer sets used in the PCR reaction may be designed to produce PCR amplified fragments containing restriction endonuclease cleavage site sequences for introduction of a feline coronavirus S gene fragment in a specific orientation into a selected expression vector to produce fusion proteins of the invention. The vector may contain a desired protein or fragment thereof to which the S gene fragment is fused in frame to produce a fusion protein.

Proteins or peptides may be selected to form fusion proteins with the selected S gene sequence based on a number of considerations. For example, a fusion partner for the S-derived fragment may be selected because it is highly expressed in the selected host cell system and may confer high expression levels on the S-derived sequence fused to it. As described in detail in Example 5 below, a selected fusion protein of this invention is produced by fusing the selected S gene sequence in frame to 52 amino acids of the bacterial enzyme, galactokinase (galK), which catalyzes the first step of galactose metabolism in bacteria. The sequence of this enzyme has been manipulated to permit insertion of foreign genes and the construction of fusion proteins. GalK is highly expressed in E. coli expression systems.

Similarly, the fusion partner may be a preferred signal sequence, a sequence which is characterized by enhanced secretion in a selected host cell system, or a sequence which enhances the stability of the S-derived peptide. Some other exemplary fusion partners which may be selected in place of galactokinase include, without limitation, ubiquitin and a mating factor for yeast expression systems, and beta-galactosidase and influenza NS-1 protein for bacterial systems. One of skill in the art can readily select an appropriate fusion partner for a selected expression system. The present invention is not limited to the use of any particular fusion partner.

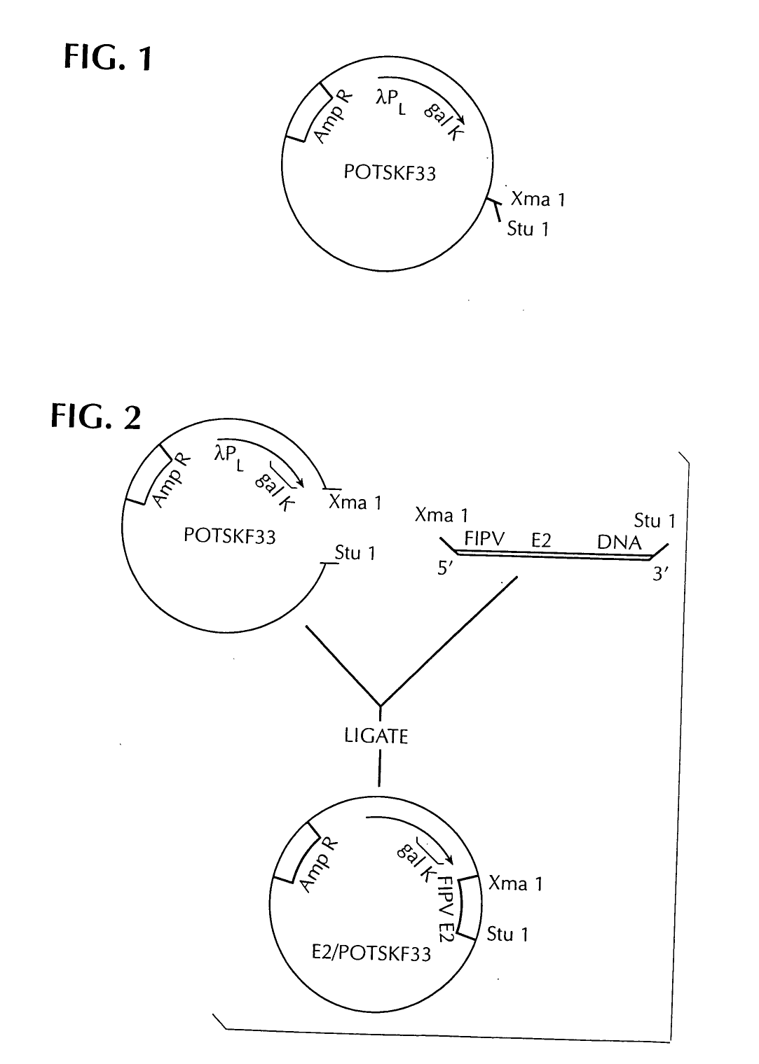

Vectors of the invention may be designed for expression of S gene pep tides or fusion proteins in bacterial, mammalian, fungal or insect cells or in selected viruses. Suitable vectors are known to one skilled in the art by resort to known publications or suppliers. The vector employed in the construction of the fusion proteins of the examples below is a bacterial pBR322-derived expression vector, pOTSKF33 (see FIG. 1 and Example 5). Plasmid pOTSKF33 is a derivative of pBR322 [Bethesda Research Laboratories] and carries regulatory signals from bacteriophage lambda. Phage regulatory information was chosen because of its high efficiency and its ability to be regulated. The system provides a promoter which can be controlled (λP_{L}), antitermination mechanisms to ensure efficient transcription across any gene insert, high vector stability, antibiotic selection, and flexible sites for insertion of any gene downstream of the regulatory sequences. The S gene sequence PCR fragments were engineered so that cloning into the unique restriction sites of pOTSKF33 (using XmaI and StuI) results in the construction of galactokinase/FIPV S peplomer fusion genes. One such fusion gene is illustrated in FIG. 2.

The resulting DNA molecules or vectors containing the sequences encoding the feline coronavirus S-derived peptides or fusion genes are then introduced into host cells and expression of the heterologous protein induced. Suitable cells or cell lines for use in expressing the S-derived peptides or fusion proteins of this invention are presently preferred to be bacterial cells. For example, the various strains of E. coli (e.g., HB101, MC1061) are well-known as host cells in the field of biotechnology. Various strains of B. subtilis, Pseudomonas, other bacilli and the like may also be employed in this method.

It is also anticipated that mammalian cells, such as Chinese hamster ovary cells (CHO) or COS-1 cells, may be used in the expression of the proteins, peptides and fusion proteins of this invention. The selection of other suitable mammalian host cells and methods for transformation, culture, amplification, screening and product production and purification are known in the art. See, e.g., Gething and Sambrook, Nature, 293:620-625 (1981), or alternatively, Kaufman et al, Mol. Cell. Biol., 5(7): 1750-1759 (1985) or Howley et al, U. S. Pat. No. 4,419,446.

Similarly many strains of yeast, or other fungal cells known to those skilled in the art are also available as host cells for expression of the proteins, peptides and fusion proteins of the present invention. Yeast expression vectors are constructed employing yeast regulatory sequences to express the DNA encoding a protein, peptide or fusion protein in yeast cells so that they yield secreted extracellular active inhibitor. [See, e.g., procedures described in published PCT application WO 86/00639 and European patent application EP 123,289.] Insect cells are also known host cells used in the expression of recombinant proteins and may be employed as host cells herein. Additional expression systems may include the known viral expression systems, e.g., vaccinia, fowlpox, swine pox. It is understood additionally, that the design of the expression vector will depend on the choice of host cell. A variety of suitable expression systems are known to those skilled in the art.

After the transformed host cells are cultured for suitable times and under suitable culture conditions known to those skilled in the art, the cells may be lysed. It may also be possible depending on the construct employed, that the recombinant proteins are secreted extracellularly and obtained from the culture medium. Cell lysates or culture medium are then screened for the presence of S-derived peptides or fusion proteins which are recognized by antibodies, preferably MAbs, to a peptide antigenic site from FIPV, FECV or consensus sequence, and in the case of a fusion protein, to the fusion partner, e.g., E. coli galactokinase.

The crude cell lysates containing the S-derived peptides or fusion polypeptides can be used directly as vaccinal components, therapeutic compositions or diagnostic reagents. Alternatively, the S-derived peptides or fusion proteins can be purified from the crude lysate or medium by conventional means. For example, galactokinase/FIPV S fusion polypeptides can be purified from bacterial lysates by affinity chromatography. Briefly, columns are prepared with monoclonal antibodies to galactokinase. The selected MAbs recognize epitopes within the first 52 amino acids of the enzyme. Bacterial lysates containing the fusion proteins are adsorbed onto the affinity matrix forming antigen-antibody complexes as the material moves through the column. After washing the column, the bound galK/S peplomer (FIPV, FECV or consensus) fusion protein is eluted by treatment with acid, base or chaotropic agents. The purified S-derived peptide or fusion protein is then more desirable for use as a vaccine component or a diagnostic reagent.

Thus the expression of the PCR amplified S gene sequence or S gene/fusion partner DNA sequences in the host cells, e.g., the galK/FIPV or FECV S fragments produced in bacterial cells, produces recombinant proteins which may be employed in diagnostic assays or as components of therapeutic and vaccinal compositions. As one example, the purified recombinant fusion protein, 58-3 (SEQ ID NOS: 19 and 20, nucleic acid and amino acid sequences, respectively), prepared according to the present invention contains a feline coronavirus S gene portion corresponding to amino acids 97 to 223 of TS FIPV. In the same manner fusion proteins may be formed with FECV amino acid sequences or amino acid sequences of the other FIP strains disclosed herein.

The recombinant proteins of this invention may thus be incorporated in a vaccine composition. Such a vaccine composition may contain an immunogenic amount of one or more selected S-derived peptides, proteins, e.g., encoded by the complete S gene sequence of FECV, or fusion proteins prepared according to the method of the present invention, together with a carrier suitable for parenteral administration as a vaccine composition for prophylactic treatment of FIPV infections. It is preferable that the recombinant protein employed in the vaccine composition contains an S gene sequence which induces protective immune responses against more than one strain of FIPV.

It is additionally desirable that the S-derived peptides, proteins or fusion proteins of this invention be employed in a vaccine composition which includes additional antigens, e.g. other coronaviruses or other pathogens in general. For example, an S-derived peptide, protein or fusion protein of the present invention may be employed as an additional antigen in the temperature sensitive FIPV vaccine described in detail in co-owned, co-pending U.S. patent application Ser. No. 07/428,796 filed Oct. 30, 1989 [SKB 14393], incorporated by reference herein. Alternatively, the peptides, proteins and fusion proteins of this invention may also be included in other feline vaccine compositions, e.g., a vaccine for feline leukemia.

The preparation of a pharmaceutically acceptable vaccine composition, having appropriate pH isotonicity, stability and other conventional characteristics is within the skill of the art. Thus such vaccines may optimally contain other conventional components, such as adjuvants and/or carriers, e.g. aqueous suspensions of aluminum and magnesium hydroxides, liposomes and the like.

The vaccine composition may be employed to vaccinate naive animals against the clinical symptoms associated with FIP. The vaccines according to the present invention can be administered by an appropriate route, e.g., by the oral, intranasal, subcutaneous, intraperitoneal or intramuscular routes. The presently preferred methods of administration are the subcutaneous and intranasal routes.

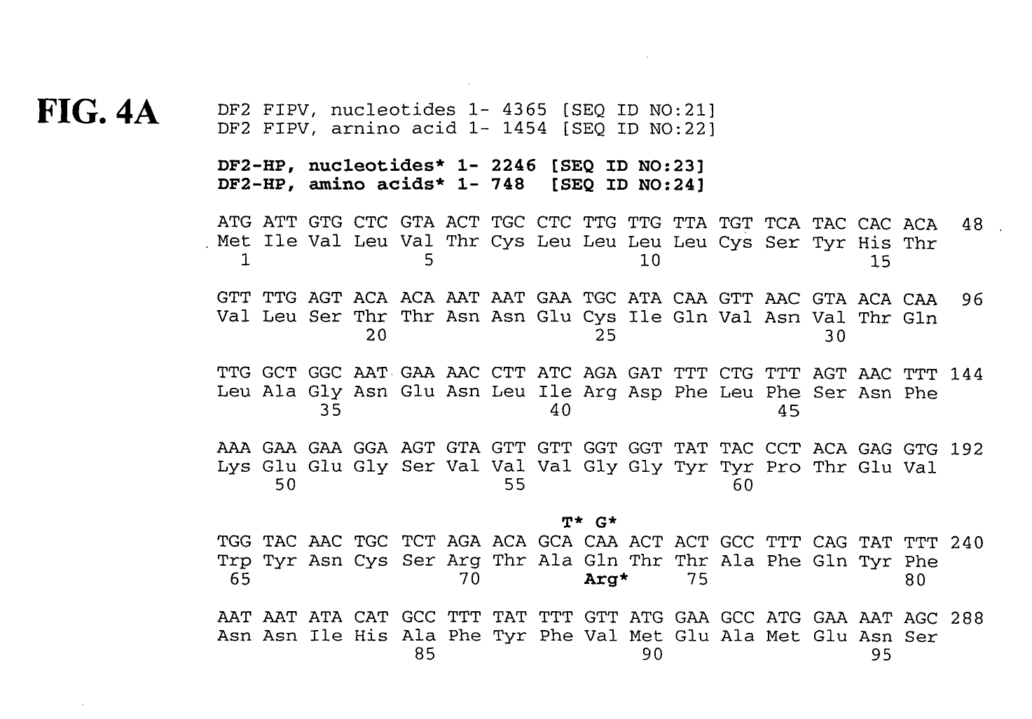

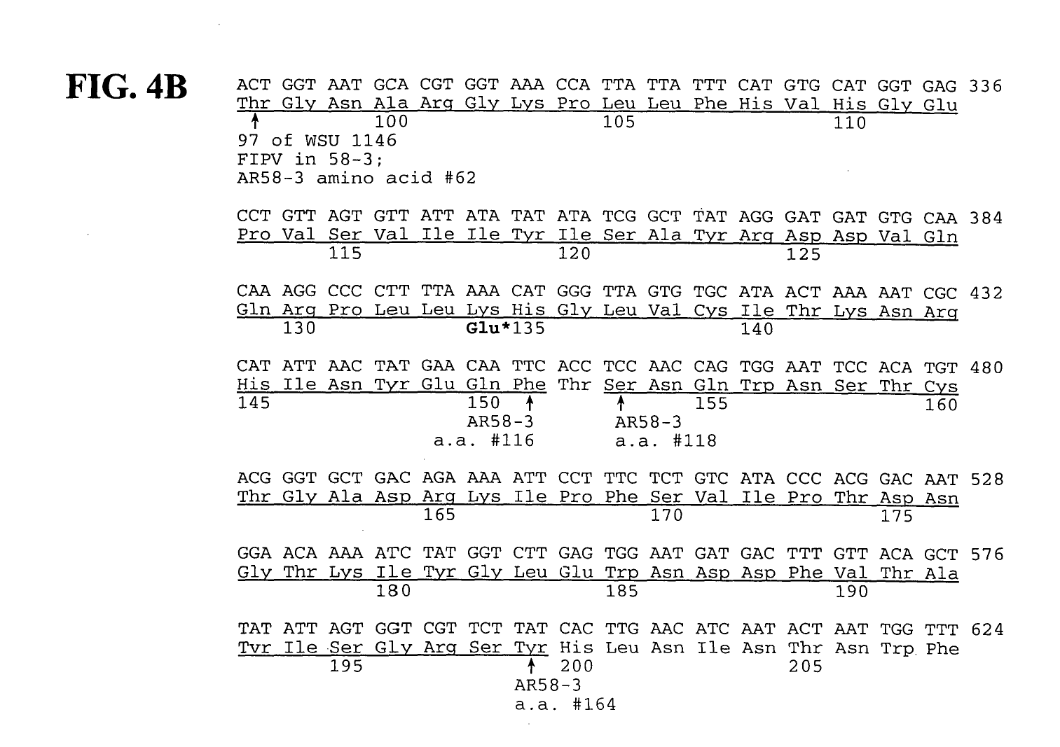

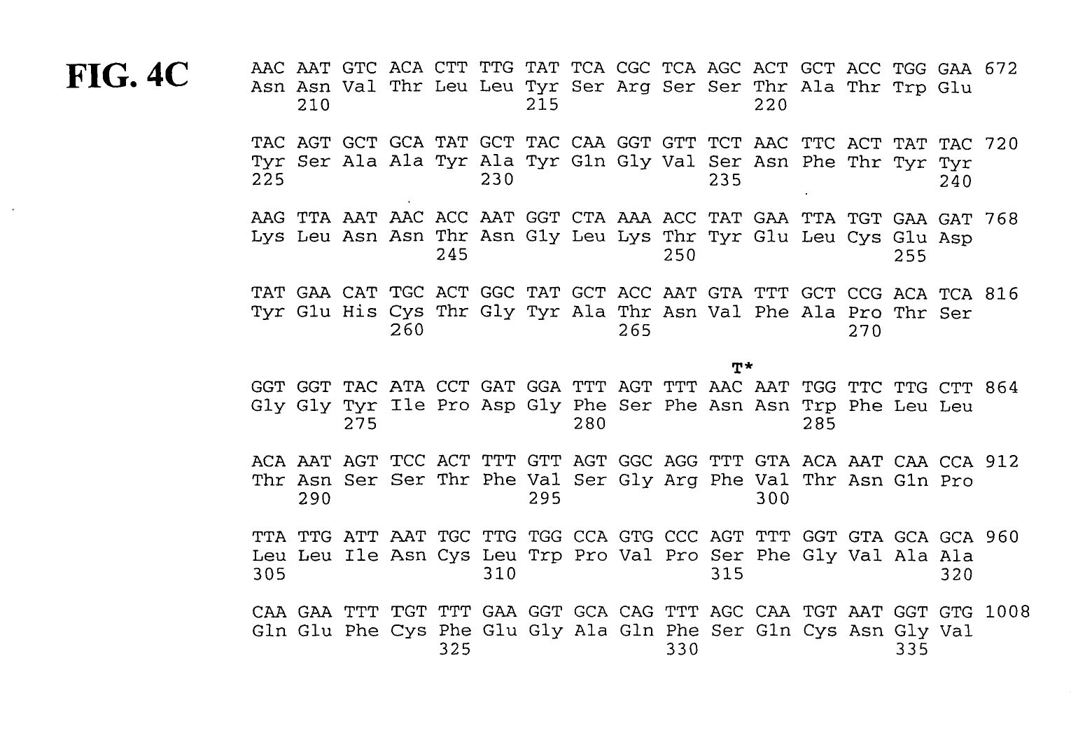

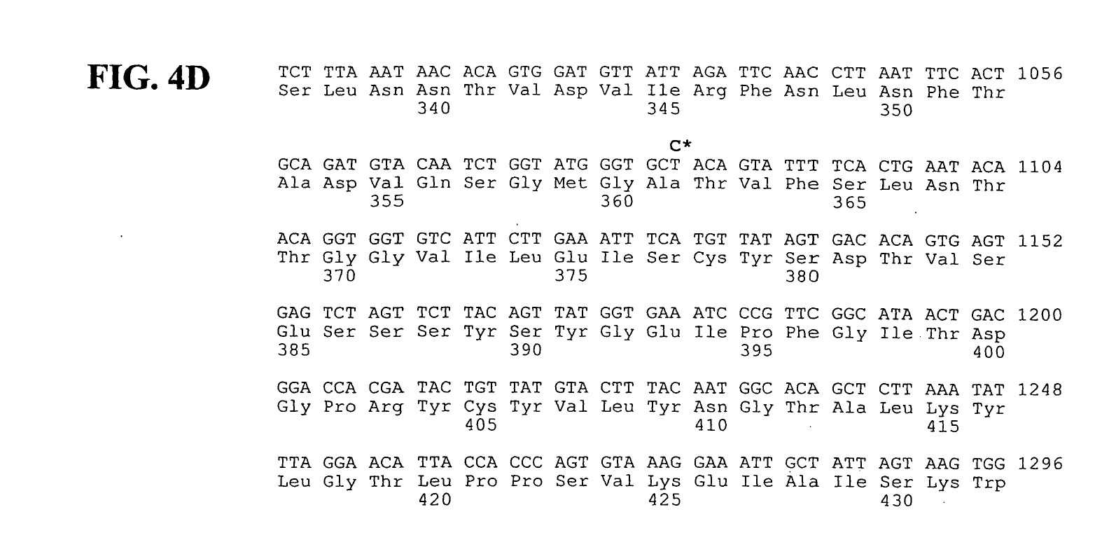

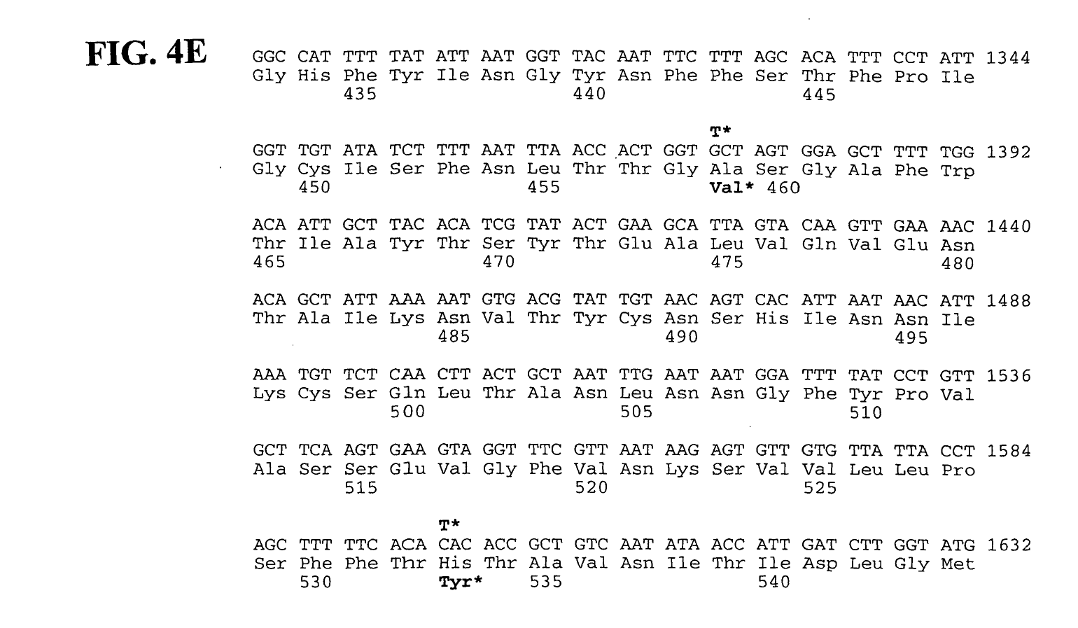

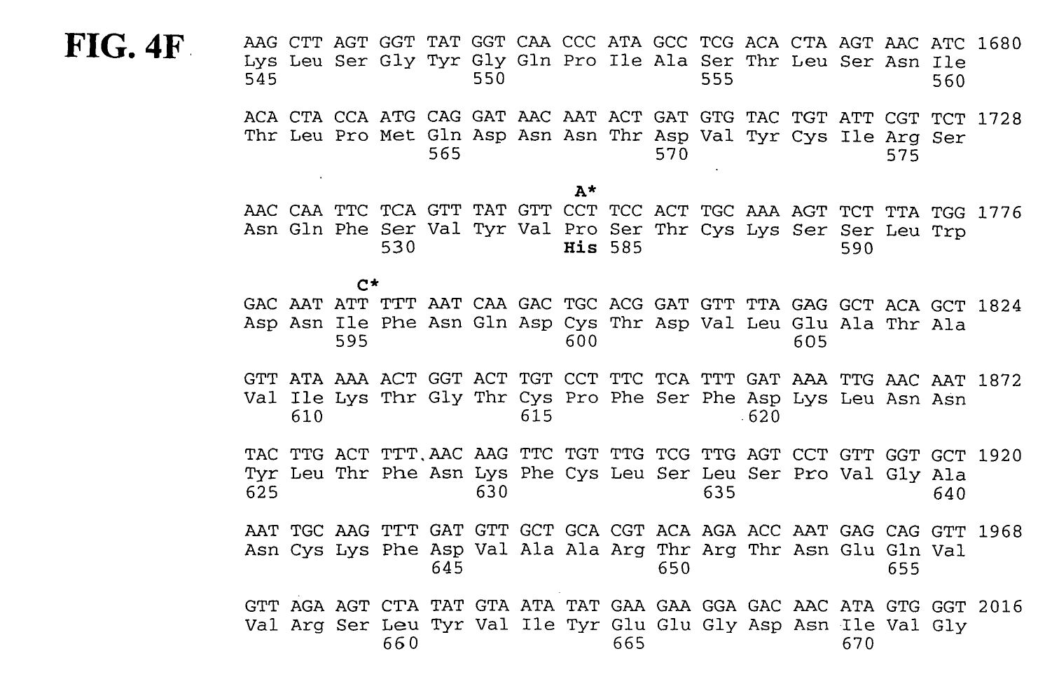

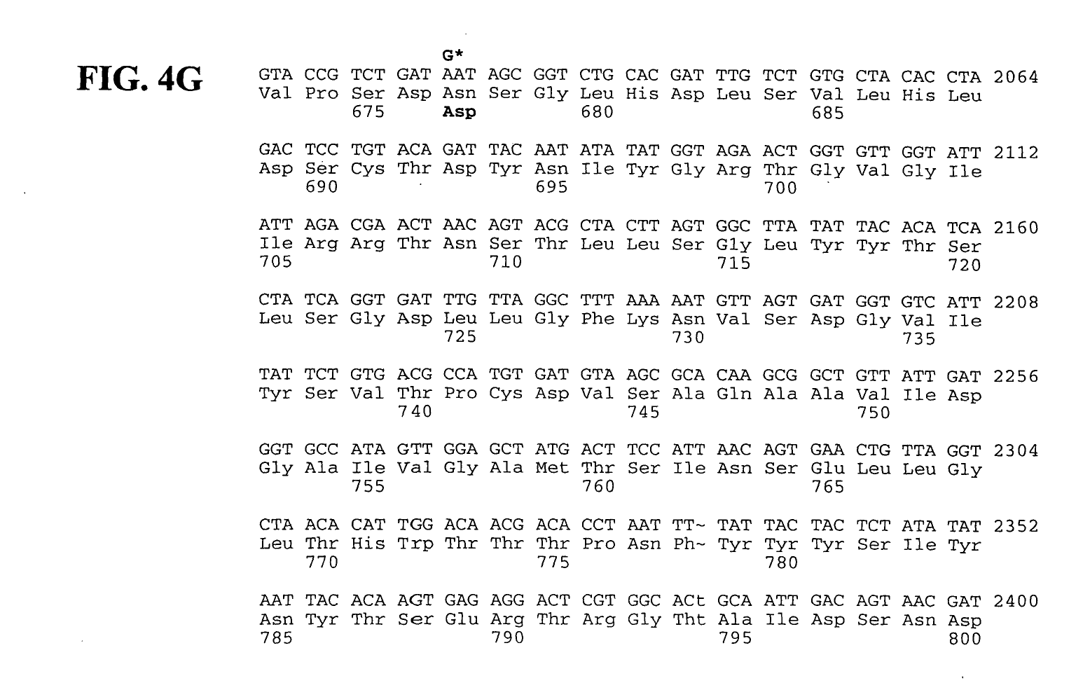

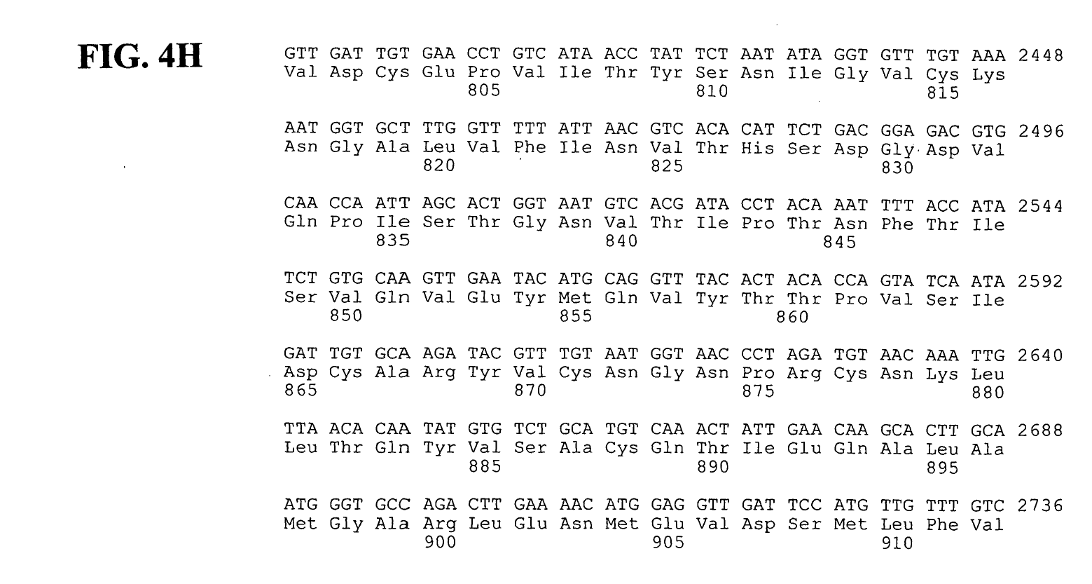

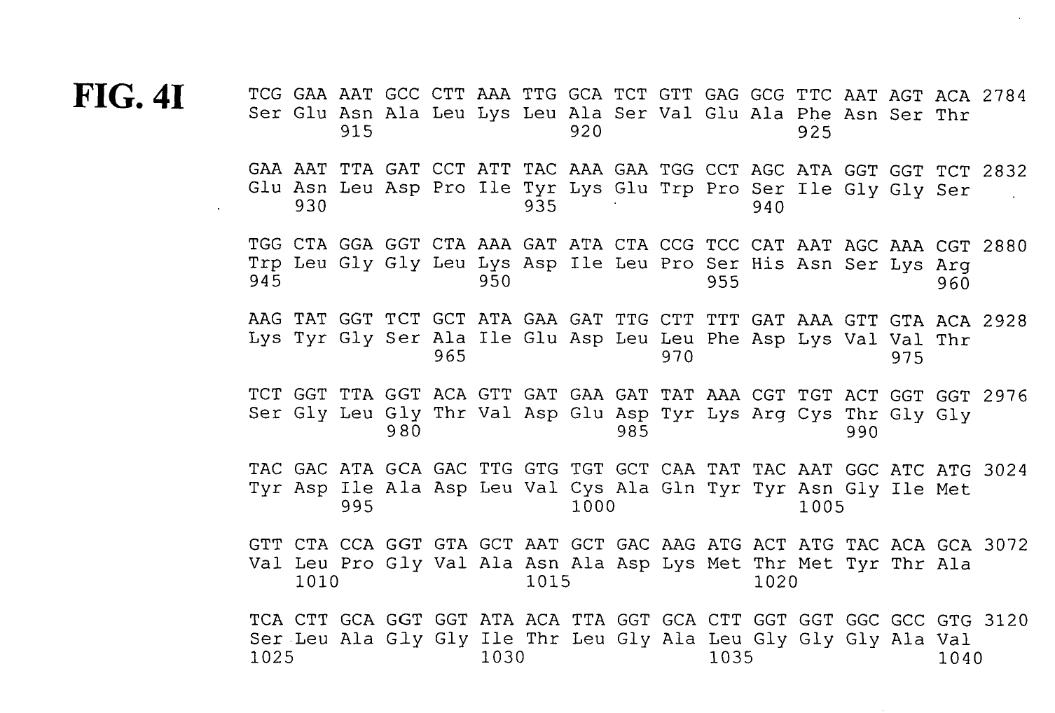

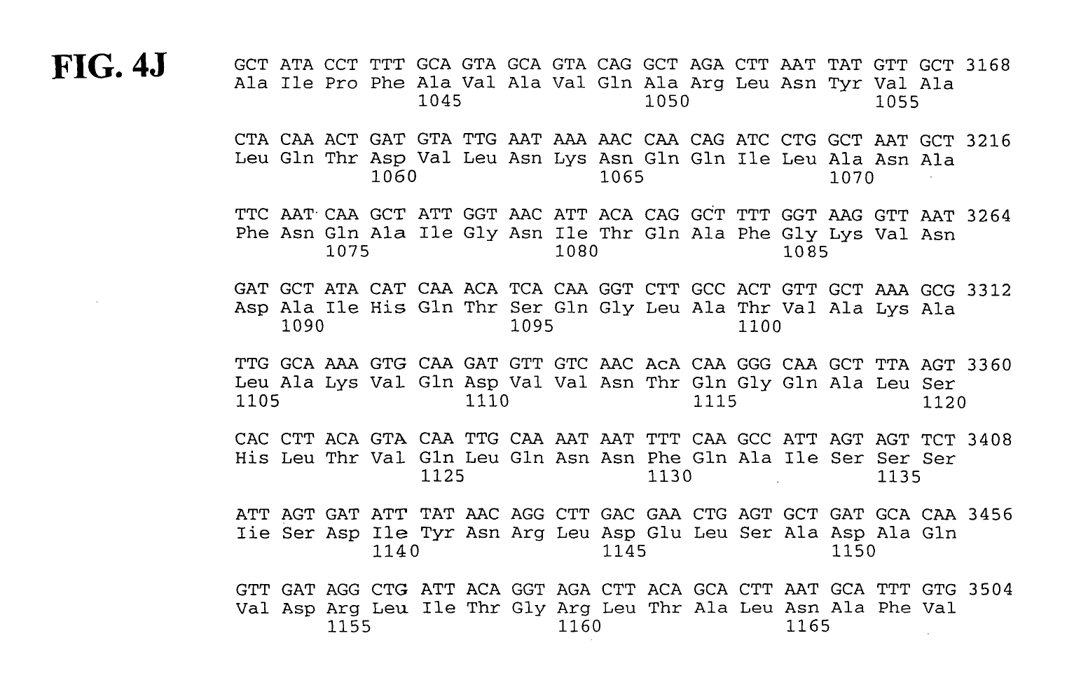

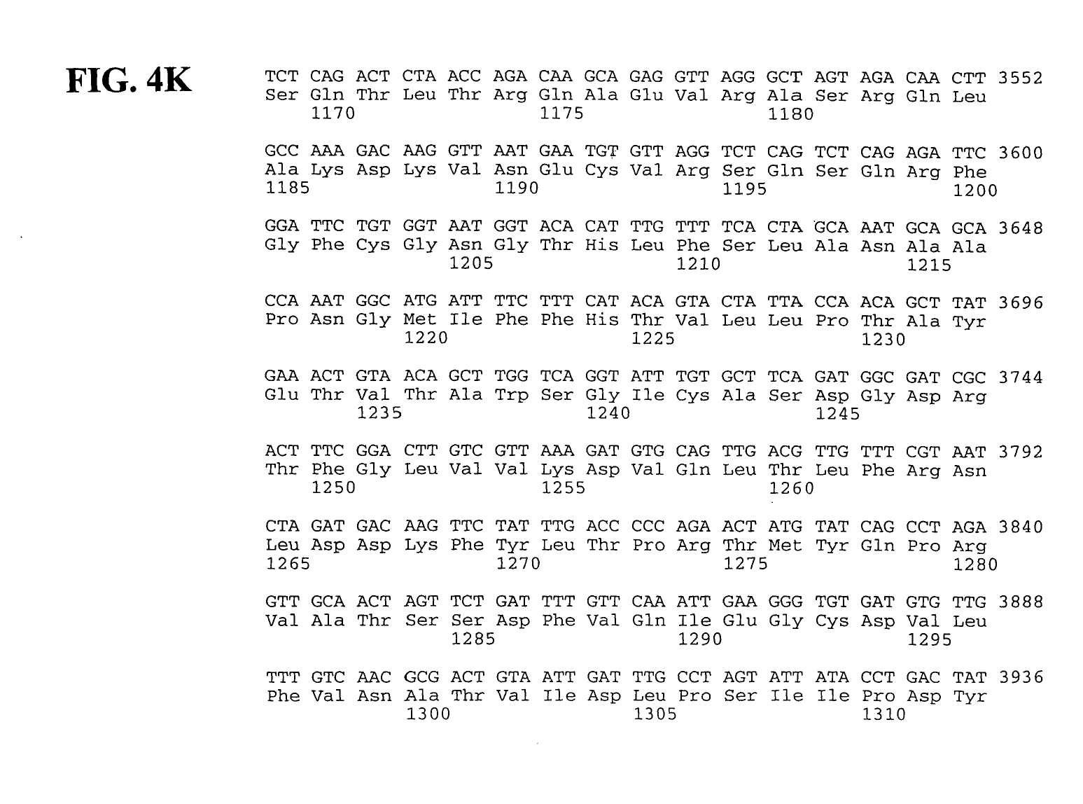

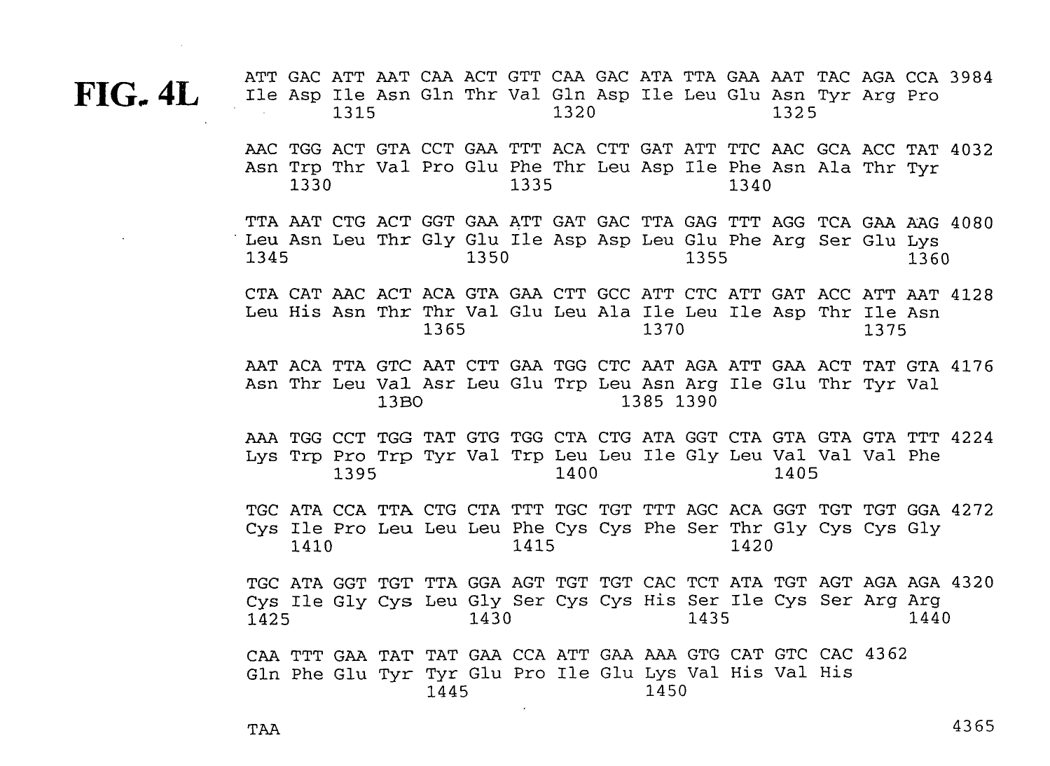

The amount of the S-derived peptide, protein or fusion protein of the invention present in each vaccine dose is selected with regard to consideration of the animal's age, weight, sex, general physical condition and the like. The amount required to induce an immunoprotective response in the animal without significant adverse side effects may vary depending upon the recombinant protein employed as immunogen and the optional presence of an adjuvant. Generally, it is expected that each dose will comprise 0.1-1000 micrograms of protein per mL, and preferably 0.1-100 micrograms per mL of a sterile solution of an immunogenic amount of a recombinant protein or peptide of this invention. Initial doses may be optionally followed by repeated boosts, where desirable. The presently preferred vaccine composition comprises at least 1-10 fusion proteins per mL. Another vaccine agent of the present invention is an anti-sense RNA sequence generated to a sequence of FIGS. 4-8. This sequence may easily be generated synthetically by one of skill in the art either synthetically or recombinantly. Under appropriate delivery, such an anti-sense RNA sequence upon administration to an infected animal should be capable of binding to the RNA of the virus, thereby preventing viral replication in the cell.

The invention also provides a pharmaceutical composition comprising S-derived peptides, proteins or fusion proteins prepared according to the present invention and a pharmaceutically effective carrier. Suitable pharmaceutically effective carriers for internal administration are known to those skilled in the art. One selected carrier is sterile saline. The pharmaceutical composition can be adapted for administration by any appropriate route, but is designed preferentially for administration by injection or intranasal administration.

The S-derived proteins, fusion proteins, or peptide fragments, as well as the PCR primers produced as described above, may also be employed in diagnostic assays which rely on recombinant derived protein immunogens as targets for sera recognition. For example, the invention provides a method of using peptides derived from the S gene of feline coronavirus, optionally fused with, e.g., the N-terminal 52 amino acids of galactokinase, as diagnostic agents useful for identifying previously exposed and naive cats, as well as for differentiating exposure to FIPV from other related coronaviruses. Other galK/FIPV S peptides or fusion proteins which show differential reactivity to FECV and FIPV sera may also be useful as FIPV-specific reagents in ELISA-based screening assays to detect FIPV exposure in cats. Similarly, an S-derived peptide or fusion protein which contained epitopes recognized only by sera from FECV infected cats or by sera from FIPV positive cats could be employed to distinguish or differentiate among coronavirus infections.

As one assay format, the reactivity of affinity purified FIPV or FECV S proteins, peptides or fusion polypeptides, e.g., galK/S fragments, to feline biological fluids or cells can be assayed by Western blot. The assay is preferably employed on sera, but may also be adapted to be performed on other appropriate fluids or cells, for example, macrophages or white blood cells. In the Western blot technique, the purified protein, separated by a preparative gel, is transferred to nitrocellulose and cut into multiple strips. The <strips are then probed with cat sera from uninfected or infected cats. Binding of the cat sera to the protein is detected by incubation with alkaline phosphatase tagged goat anti-cat IgG followed by the enzyme substrate BCIP/NBT. Color development is stopped by washing the strip in water.

Western blot screening of cat sera samples has been performed with the purified recombinant fusion protein, 58-3 (SEQ ID NO: 20), prepared according to the present invention and as described in detail in Examples 5 through 7. The feline coronavirus S gene portion of this recombinant protein is obtained from TS FIPV and corresponds to amino acids 97 to 223 of the published WSU 1146 strain. When screened with a battery of cat sera, only sera-of those cats which were sick and/or dying with DF2 or WSU 1146 FIPV reacted with the 58-3 polypeptide (SEQ ID NO: 20). Healthy cats did not react to this peptide nor did cats which were challenged with the nonvirulent FECV coronavirus strain. Other peptides of this invention may be employed similarly to distinguish between FIPV strains and FECV, or among different strains of FIPV.

Fusion protein 58-3 (SEQ ID NO: 20) may also be used in an ELISA based assay for detecting FIPV disease. Other S derived peptides or fusion proteins which show differential reactivity to FECV and FIPV sera may also be useful as FIPV-specific reagents in ELISA-based screening assays to detect FIPV exposure in cats.

A typical ELISA protocol would involve the adherence of antigen (e.g., a recombinant galK/S fusion protein) to the well of a 96-well tray. The serum to be tested is then added. If the serum contains antibody to the antigen, it will bind. Specificity of the reaction is determined by the antigen absorbed to the plate. With the 58-3 galK/FIPV S fusion protein (SEQ ID NO: 20), only sera from those cats sick or dying from FIPV would bind to the plate; sera from naive or healthy virus-exposed cats would not bind.

Similarly, an S-derived protein, peptide or fusion protein which contained epitopes recognized only by sera from FECV infected cats or by sera from FIPV positive cats could be employed to distinguish coronavirus infections. After the primary antibody is bound, an enzyme-labelled antibody directed against the globulin of the animal whose serum is tested is added. Substrate is then added. The enzyme linked to antibody bound to the well will convert the substrate to a visible form. The amount of color measured is proportional to the amount of antibody in the test material. In this manner, cats previously infected with FIPV can be identified and treated, or cats naive to the virus can be protected by vaccination.

The present invention also encompasses the development of an antibody to one of the above identified amino acid residue regions of FECV or to fusion proteins carrying such a region, which region does not react with other coronavirus, e.g. FIPV. In one embodiment, the antibody is capable of identifying or binding to an FECV antigenic site encoded by all or a portion of the DNA sequences identified below in FIGS. 3-8. Such an antibody may be used in a diagnostic screening test or as therapeutic agents.

Antibodies to peptides of the regions identified above or to other regions capable of distinguishing between FIPV and FECV for use in the assays of this invention may be polyclonal. However, it is desirable for purposes of increased target specificity to utilize monoclonal antibodies (MAbs), both in the assays of this invention and as potential therapeutic and prophylactic agents. Additionally, synthetically designed monoclonal antibodies may be made by known genetic engineering techniques [W. D. Huse et al, Science, 246:1275-1281 (1989)] and employed in the methods described herein. For purposes of simplicity the term MAb(s) will be used throughout this specification; however, it should be understood that certain polyclonal antibodies, particularly high titer polyclonal antibodies and recombinant antibodies, may also be employed.

A MAb may be generated by the well-known Kohler and Milstein techniques and modifications thereof and directed to one or more of the amino acid residue regions identified above, or to other FECV-encoded peptides or epitopes containing differences between itself and FIPV, such as those identified in Example 12 below. For example, such a portion of the FECV sequence encoding an antigenic site, which differs from that of FIPV, may be presented as an antigen in conventional techniques for developing MAbs. A cell line secreting an antibody which recognizes an epitope of FECV only, not on FIPV or any other coronavirus, may then be identified for this use. One of skill in the art may generate any number of MAbs by using fragments of the amino acid residue regions identified herein as an immunogen and employing these teachings.

For diagnostic purposes, the antibodies (as well as the diagnostic probes) may be associated with individual labels, and where more than one antibody is employed in a diagnostic method, the labels are desirably interactive to produce a detectable signal. Most desirably, the label is detectable visually, e.g. colorimetrically. Detectable labels for attachment to antibodies useful in the diagnostic assays of this,invention may also be easily selected by one skilled in the art of diagnostic assays. Labels detectable visually are preferred for use in clinical applications due to the rapidity of the signal and its easy readability. For colorimetric detection, a variety of enzyme systems have been described in the art which will operate appropriately. Colorimetric enzyme systems include, e.g., horseradish peroxidase (HRP) or alkaline phosphatase (AP). Other proximal enzyme systems are known to those of skill in the art, including hexokinase in conjunction with glucose-6-phosphate dehydrogenase. Also, bioluminescence or chemiluminescence can be detected using, respectively, NAD oxidoreductase with luciferase and substrates NADH and FMN or peroxidase with luminol and substrate peroxide. Other conventional label systems that may be employed include fluorescent compounds, radioactive compounds or elements, or immunoelectrodes. These and other appropriate label systems and methods for coupling them to antibodies or peptides are known to those of skill in the art.

Antibodies specific for epitopes on FIPV, which are not capable of binding FECV, or alternatively which are specific to epitopes on virulent strains of FIPV but not avirulent strains, may also be used therapeutically as targeting agents to deliver virus-toxic or infected cell-toxic agents to infected cells. Rather than being associated with labels for diagnostic uses, a therapeutic agent employs the antibody linked to an agent or ligand capable of disabling the replicating mechanism of the virus or of destroying the virally-infected cell. The identity of the toxic ligand does not limit the present invention. It is expected that preferred antibodies to peptides encoded by the sequences identified herein may be screened for the ability to internalize into the infected cell and deliver the ligand into the cell.

The assay methods, PCR primers, S-derived proteins, peptides and fusion proteins and antibodies described herein may be efficiently utilized in the assembly of a diagnostic kit, which may be used by veterinarians. The kit would be useful in distinguishing between native FIPV exposed animals and vaccinated animals, as well as non-exposed cats, and between FIPV-infected animals and animals infected with serologically related viruses, such as FECV. Such a diagnostic kit contains the components necessary to practice the assays described above.

Thus, the kit may contain a sufficient amount of at least one fusion protein or at least one S gene protein or peptide or PCR primer pair of this invention, a MAb directed to a first epitope on the FIPV S fragment, (which Mab may be labeled), optional additional components of a detectable labelling system, vials for containing the serum samples, protein samples and the like, and a second mAb conjugated to the second enzyme, which in proximity to the first enzyme, produces a visible product. Other conventional components of such diagnostic kits may also be included.

Alternatively, a kit may contain a selected FIPV S peptide or fusion protein, a Mab directed against a selected FIPV S peptide fragment bound to a solid surface and associated with a first enzyme, a different MAb associated with a second enzyme, and a sufficient amount of the substrate for the first enzyme, which, when added to the serum and MAbs, provides the reactant for the second enzyme, resulting in the color change.

Other known assay formats will indicate the inclusion of additional components for a diagnostic kit according to this invention.

The examples which follow are intended as illustrative only and do not limit the scope of the present invention.

### EXAMPLE 1

Prediction of Potential Antigenic Sites

The computer program developed by Jameson and Wolf, Cabios, 4:181-186 (1988) was used to predict potential antigenic sites on the amino acid sequence of the published FIPV WSU 1146 strain (available upon request from the Washington State University). This program was designed to integrate the influence of five major factors that historically have been important in accurate prediction of antigenic sites. Hydrophilicity values are determined according to Hopp and Woods, Proc. Natl. Acad. Sci. USA, 78:3824-3828 (1981). Potential surface probabilities are primarily determined by the method of Janin et al, J. Mol. Biol., 125:357-386 (1978), but more recently modified according to Emini et al, J. Virol., 55:836-839 (1985).

Backbone flexibility of the protein was determined as described by Karplus and Shultz, Naturwissenschaften, 72:212-213 (1985), while prediction of protein secondary structure was computed by two methods. The algorithm of Chou and Fasman, Adv. Enzymol., 47:145-147 (1978) as modified by Nishikawa Biochim. Biophys Acta, 748:285-299 (1983) to include overall probability, was the first method used for secondary structure prediction. In addition, a program developed by Garnier et al, J. Mol. Biol., 120:97-120 (1978) was used in support of Chou-Fasman. The greatest accuracy of secondary structure prediction occurs at points where the two different subroutines are in agreement [Jameson and Wolf, supra).

Each of these factors are computed in concert to produce a summary value, the antigenic index. Output of the program was plotted in linear fashion along the amino acid sequence of the S gene. Analysis of the FIPV S protein was performed on a host computer consisting of a Vax 8800 series (Digital Equipment Corporation) cluster running under the VMS operating system. These programs are available as part of the University of Wisconsin Computer Group (GCG) package environment [Devereux, Nucleic Acids Research, 12:387-395 (1984)].

This analysis of the protein sequence using the WT WSU 1146 and TGE coronavirus sequences showed that the FIPV S protein is conserved in the C terminus (⅔ of gene) while variation was concentrated in the N-terminus (⅓ of gene). As predicted by computer analysis, there is little differentiation of the carboxy terminus of the S gene.

### EXAMPLE 2

Oligonucleotide Design

Oligonucleotides were designed to divide the WSU 1146 S gene of 4500 base pairs (1452 amino acids) into approximately 300-500 base pair fragments. Each of these fragments was chosen to encompass one or more major antigenic peaks as determined from the computer analysis described above. Primers were typically 30-40 base pairs in length and included an XmaI restriction site in the upstream (5′) primer and a StuI restriction site in the downstream (3′) primer. [See Table I below, SEQ ID NOS: 1-18]. These sites were incorporated into the primers to allow for directional, in-frame cloning into the expression vector.

In addition, five additional FIPV matching base pairs were added upstream of each restriction site in order to stabilize the DNA-RNA hybrid and allow amplification to occur efficiently. The oligonucleotides were designed to have a relatively high G-C content (approximately 50% or greater) which provided additional stability to the hybrid.

Primer sequences were compared by computer against the published WSU 1146 sequence to insure that they only primed a specific area, did not form “primer dimer” structures with other primers and had no internal secondary structure that could inhibit proper hybridization with the coronavirus RNA/DNA during amplification.

Table II illustrates the FIPV S oligonucleotide primers amplified by PCR technique, 5′ through 3′ (SEQ ID NOS: 1-18). These primers, designed as described above, were synthesized on an Applied Biosystem Model 380B DNA Synthesizer by the phosphoramidite method, and were gel purified prior to use. At nucleotide #6-11, primer SEQ ID NOS: 1-9 contain an Xma site (CCCGGG) and primer SEQ ID NOS: 10-18 contain an Stu I site.

These primers used for the PCR amplification and resulting fusion proteins of this invention may contain stop codons after fusion with GalK. However, effective binding of the oligonucleotide is important for effective PCR priming, which is not dependent on expression and is therefore unaffected by the presence of a stop codon in the primer.

Nucleotides may be changed at the primer level to eliminate the stop codon problem, and several such changes are indicated in Table II below by asterisks. For example, the second, third, fifth, sixth and ninth 5′ primers (SEQ ID NOS: 2, 3, 5 and 9, respectively) may be changed by deleting the nucleotide below the asterisk in each sequence. The fourth and eighth 5′ primers (SEQ ID NOS: 4 and 8, respectively) may be changed by adding a T or A, respectively, in front of the nucleotide marked with an asterisk. Additionally, DNA sequence can be added, deleted, or altered as a result of PCR and/or gene expression in bacteria. Therefore, the sequence of all clones must be verified to detect errors in sequence. Any sequence errors can be corrected at the nucleotide level in the expression clones by one of skill in the art with resort to conventional techniques.

The PCR data so far has been obtained using the Table II primers (SEQ ID NOS: 1-18). These same primers were also used in making the expression fusion proteins described in detail below. However, some of the resulting clones were corrected to obtain effective expression data.

TABLE II

| | | |
|---|---|---|
| Position (BP) | Position (AA) | Sequence |

5′ (sequence same polarity as published WSU 1146, contains Xma site)

| | | |
|---|---|---|
| 5′ Xma/3′ Xma | | |
| | | Xma |
| 65-69/70-96 (Start) | 1-9 | GTGCCCCCGGGTATGATTGTGCTCGTAACTTGCCTCTTG |
| SEQ ID NO:1 | | start codon |
| | | * |
| 351-355/356-380 | 95-104 | AATACCCGGGGCACTGGTAATGCACGTGGTAAACC |
| SEQ ID NO:2 | | |
| | | * |
| 705-709/710-733 | 213-219 | GTATTCCCGGGCACGCTCAAGCACTGCTACCTGGG |
| SEQ ID NO:3 | | |
| | | * |
| 1121-1125/1126-1150 | 352-360 | CAGATCCCGGGGTACAATCTGGTATGGGTGCTACAG |
| SEQ ID NO:4 | | |
| | | * |
| 1698-1702/1703-1730 | 544-554 | GCTTACCCGGGGTGGTTATGGTCAACCCATAGCCTCGAC |
| SEQ ID NO:5 | | |
| | | * |
| 2277-2281/2282-2309 | 737-746 | TGTGACCCGGGCGCCATGTGATGTAAGCGCACAAGCGGC |
| SEQ ID NO:6 | | |
| 2749-2753/2754-2779 | 894-903 | GCAATCCCGGGGGGTGCCAGACTTGAAAACATGGAGG |
| SEQ ID NO:7 | | |
| | | * |
| 3155-3159/3160-3185 | 1030-1038 | CATTACCCGGGGGTGCACTTGGTGGTGGCGCCGTGGC |
| SEQ ID NO:8 | | |
| | | * |
| 3642-3646/3647-3674 | 1192-1201 | TAGGTCCCGGGCTCAGTCTCAGAGATTCGGATTCTGTGG |
| SEQ ID NO:9 | | |

3′ (sequence reverse complement of published WSU 1146, contains Stu I site)

| | | |
|---|---|---|
| 5′ StuI/3′ Stu I | | |
| | | Stu I |
| 385-381/380-356 | 97-105 | ATAATAGGCCTGGTTTACCACGTGCATTACCAGTGC |
| SEQ ID NO:10 | | |
| 738-734/733-710 | 213-223 | GTATTAGGCCTCCCAGGTAGCAGTGCTTGAGCGTG |
| SEQ ID NO:11 | | |
| 1155-1151/1150-1126 | 353-362 | AAATAAGGCCTCTGTAGCACCCATACCAGATTGTAC |
| SEQ ID NO:12 | | |
| 1735-1731/1730-1703 | 546-555 | TTAGTAGGCCTGTCGAGGCTATGGTTGACCATAACCAC |
| SEQ ID NO:13 | | |
| 2314-2310/2309-2282 | 739-748 | TAACAAGGCCTGCCGCTTGTGCGCTTACATCACATGGCG |
| SEQ ID NO:14 | | |
| 2784-2780/2779-2754 | 896-905 | ATCAAAGGCCTCCTCCATGTTTTCAAGTCTGGCACCC |
| SEQ ID NO:15 | | |
| 3190-3186/3185-3160 | 1031-1040 | GTATAAGGCCTGCCACGGCGCCACCACCAAGTGCACC |
| SEQ ID NO:16 | | |
| 3679-3675/3674-3647 | 1194-1203 | CATTAAGGCCTCCACAGAATCCGAATCTCTGAGACTGAG |
| SEQ ID NO:17 | | |
| 4433-4429/4428-4405 (Stop) | 1444-1452 | TAAATAGGCCTTTAGTGGACATGCACTTTTTCAATTGG |
| SEQ ID NO:18 | | *stop codon |

### EXAMPLE 3

Preparation of RNA and cDNA for PCR

The RNA which was used as a template for generation of the PCR amplified fragments useful in this invention was obtained from the following coronavirus strains: WT WSU 1146 and FECV (WSU 1683) from Washington State University, WT UCD-1, WT-UCD-2, and WT UCD-4 from N. Pedersen at the University of California-Davis, WT TN406 from Dr. J. Black, Tennessee, and WT DF2 and TS DF2 from SmithKline Beecham Animal Health, Lincoln. WT UCD-1, WT WSU 1146, and WSU 1683 are available from the American Type Culture Collection, Rockville, Maryland. The other strains are available upon request from their respective suppliers.

Viruses were cultivated as follows. Roller bottles of confluent Norden Laboratories feline kidney (NLFK) cells were infected with either WT DF2, WT WSU 1146 or FECV 1683 virus using the following protocol. The WT DF-2 FIP virus was originally isolated from a cat liver explant. After several passages of tissue homogenates in specific pathogen free (SPF) cats, the virus was adapted to Norden Laboratory Feline Kidney (NKLF) cells by cocultivation with infected primary spleens.

The TS DF-2 virus mutant was derived from WT DF2 FIP virus which had been passaged 60 times on NKLF cells at 39° C. followed by 39 passages at 31° C. The virus collected at pass 99 was ultraviolet irradiated for 5 minutes and then plaque purified prior to use as described in Christianson et al, Arch. Virol., 109:185-196 (1989).

The growth medium was removed and virus (MOI=0.1) was absorbed in 50 ml of BME supplemented with 2% FBS. Only WT DF2 FIPV infections were performed in serum-free medium. The virus was absorbed for 2 hours and then 250 ml of growth medium added. The cultures were monitored for cytopathic effect (CPE) and typically harvested at 24-36 hours post-infection.

A similar protocol was followed for infections with the TS FIPV strain except all incubations were performed at 31° C.

WT TN406, WT UCD-1 and WT UCD-2 were grown in T150 flasks of Felis catus whole fetus (FCWF) cells. Cells were split 1:2 and inoculated with approximately 10⁵ TCID₅₀ of virus in 50 ml of BME+2% FBS. The cultures were again monitored for CPE and typically harvested at 48-72 hours post-infection.

Total cytoplasmic RNA was prepared from the infected monolayers by guanidine isothiocyanate extraction according to Chirgwin, Biochemistry, 18:52.94 (1979). Where indicated, poly A+ mRNA was isolated from total RNA by absorption to and subsequent batch elution from oligo dT cellulose. The cDNA was synthesized from this total RNA by standard techniques.

### EXAMPLE 4

PCR Amplification

PCR amplification was performed on the cDNA of the FIPV of Example 3 under the following conditions:

In a final reaction volume of 20 μl of 1×PCR buffer (10×PCR buffer: 100 mM Tris-HCl, 500 mM KCl, 15 mM MgCl₂, 0.01% (w/v) gelatin) the following components were assembled in RNAse free siliconized 500 μl microcentrifuge tubes: 1.0 mM of DATP, dCTP, dGTP and dTTP (dNTPs), 20 units of RNAsin (Promega Corp), 100 picomoles of random hexamer oligonucleotides (Pharmacia, 100 picomoles/μl solution in TE buffer (10 mM Tris-HCl, 1 mM EDTA, pH 7.5)), 200 units of reverse transcriptase (Moloney MuLV, Bethesda Research Labs) and 1.0 μg of respective RNA isolated as described above.

To avoid pipetting errors and contamination, all solutions were aliquoted from master mixes made with diethyl pyrocarbonate (DEPC) treated water and consisted of all of the reaction components except the RNA which was added last. The mixture was incubated in a programmable thermal cycler (Perkin-Elmer Cetus) at 21° C. for ten minutes followed by 42° C. for one hour, then 95° C. for five minutes and finally held at 4° C. until PCR amplification.

Amplification of the cDNA was performed essentially according to the method of Saiki et al, Science, 230:1350-1354 (1985) using the Taq polymerase. Briefly, to the 20 μL cDNA reaction mixture from above was added 8.0 μL 10×PCR buffer, 1.0 μL of each upstream and downstream primer previously diluted in water to 30 picomoles per microliter and 5.0 units of Taq polymerase (Perkin-Elmer Cetus). Final volume was made up to 100 μL using DEPC (diethyl pyrocarbonate) treated water and overlaid with 100 μL of mineral oil. As above master mixes were prepared to avoid contamination.

The reaction was performed in the Perkin-Elmer Cetus thermal cycler for one cycle by denaturing at 95° C. for 1 minute, annealing at 37° C. for 2 minutes followed by extension at 72° C. for 40 minutes. This initial cycle increased the likelihood of first strand DNA synthesis. A standard PCR profile was then performed by a 95° C.-1 minute denaturation, 37° C.-2 minute annealing, 72° C.-3 minute extension for 40 cycles. A final extension profile was done by 95° C.-1 minute denaturation, 37° C.-2 minute annealing, 72° C.-15 minute extension and held at 4° C. until analyzed.

A small aliquot (5 μl) of the completed PCR reactions were analyzed by agarose gel electrophoresis to confirm amplification of the predicted DNA fragment.

For the galK/FIPV S clone 58-3 only (SEQ ID NO: 19 and 20) [See FIG. 3], double stranded cDNA was first synthesized using 2 mg poly A+ mRNA isolated from TS FIPV infected NLFK cells. Boehringer Mannheim's cDNA synthesis kit was used according to the manufacturer's specifications. The cDNAs were extracted with phenol/chloroform (1:1), ethanol precipitated and sized on 1.4% alkaline agarose gels. The yield of cDNA was determined as specified by Boehringer.

In the PCR reaction then, 100 ng of cDNA and 100 ng of each primer were added to all 4 dNTPs, MgCl₂ and 5 units Taq polymerase in a 100 μL standard reaction mixture at concentrations as described above (see Table II]. The mixture was overlaid with 100 μL mineral oil and incubated in a Perkin Elmer Cetus thermocycler for 30 cycles. Each complete cycle incubated the samples at 94° C. for 1 minute, followed by 37° C. for 2 minutes, and ending at 72° C. for 3 minutes.

PCR amplified products were analyzed by electrophoresing 5.0 μl of the mix on a 1.2% agarose gel run overnight. Bands were visualized by ethidium bromide staining the gel and UV fluorescence. Photography using Polaroid type 55 film provided a negative that could be digitized for sample distance migration and comparison against markers run on each gel. The actual sizes of the bands were then calculated using the Microgenie (Beckman) software running on an IBM AT. Reactions distinguishing WT WSU 1146 or WT DF2 from WT UCD-1 and FECV are described below in Table III.

TABLE III
S Regions (aa) Differentiated by PCR

| Virus | 1-555 | 352-555 | 894-1452 |
|---|---|---|---|
| WT WSU 1146 or DF2 | + | + | + |
| WT UCD-1 | 0 | + | 0 |
| FECV | 0 | + | + |

The results presented in Table III indicate that the 5′ primer starting at position 1 is not able to efficiently initiate DNA synthesis from any template except. WT WSU 1146 and WT DF2. However, the 5′ primer starting at position 352 works on all strain templates. The 3′ primers starting at position 555 prime efficiently on all strains shown. The 5′ and 3′ primers at position 894 and 1452, respectively, prime DNA synthesis from WT WSU 1146, WT DF2 and FECV template, but not WT UCD-1. in this manner different strains of feline coronavirus can be distinguished.

The results of PCR amplification showed the amplification of amino acid range 737-1452 for the WT DF2, TS and FECV strains, respectively. A fragment of predicted size (2168 bp) was obtained from each virus. Amplification of a second and smaller region (amino acid range 1029-1452) provided additional evidence of similarity among the strains. A fragment of predicted size (1290 bp) was again obtained from WT DF2, TS and FECV viral templates.

The differences among the strains can be demonstrated by amplification of sites within the amino terminus. Results showed amplification of amino acid range 1-748 for WT DF2 and TS. A fragment of predicted size (2261 bp) was obtained. Repeated attempts to amplify the same region from the FECV virus yielded no fragment. In addition, PCR of the amino acid range 1-223 demonstrated that the correct fragment was obtained (685 bp) for the WT DF2 and TS strains, but extra fragments were obtained for the FECV virus. Other S gene sequences generated by PCR for each virus strain are listed in Table IV below.

### EXAMPLE 5

Cloning FIPV S Regions

The E. coli-derived vector, pOTSKF33, was chosen for the cloning of the FIPV peplomer fragments generated by PCR. Cloning procedures were as described by T. Maniatis et al, cited above. The bacterial expression vector, pOTSKF33, shown schematically in FIG. 1, is being maintained at SmithKline Beecham Laboratories and is available to the public through the company.

This plasmid is a derivative of pBR322 [Bethesda Research Laboratories] and carries regulatory signals from bacteriophage lambda. The system provides a promoter which can be controlled (λP_{L}), and an antitermination mechanism to ensure efficient transcription across any gene insert, high vector stability, antibiotic selection, and flexible sites for insertion of any gene downstream of the regulatory sequences. The pOTSKF33 vector also contains the coding sequence for 52 amino acids of the enzyme galactokinase, immediately adjacent to the λP_{L} promoter. The sequence of this enzyme has been manipulated to permit insertion of foreign genes and the construction of fusion proteins.

Linkers containing restriction sites for fusion in any of the three reading frames, stop codons for each phase and some additional cloning sites for fusion in any of the three reading frames, have been introduced after the first 52 amino acids of galactokinase.

Transcription from the P_{L} promoter is tightly controlled by maintaining the plasmid in bacteria expressing the c1⁺ repressor protein. Induction of foreign protein expression is obtained by removing the repressor. In the bacterial strains used in this study, the repressor protein is temperature-sensitive. At the permissive temperature, 32° C., the repressor functions normally to inhibit transcription from the P_{L} regulatory sequences. An increase in growth temperature (to 42° C.) results in degradation of the repressor and expression of the fusion polypeptide is induced.

In some cases, fusion proteins can represent up to 20% of total bacterial protein. These fusion proteins can be detected with monoclonal antibodies to galK.

The method for cloning of an illustrative galK/FIPV S fusion protein 58-3 (SEQ ID NO: 20) is described as follows: The mineral oil overlay was removed from the PCR reaction mixture and a 100 μl DNA fraction was digested with XmaI and StuI in a 300 μl final volume for 18 hours at 37° C. The digested DNA was first extracted with phenol followed by phenol/chloroform (1:1) and then ethanol precipitated at −20° C. XmaI/StuI digested DNAs were incubated at 15° C. for 24 hours in a ligation mixture containing pOTSKF33 vector DNA which was digested with XmaI/StuI and phosphatased.

E. coli HB101 cells were transformed and insert-bearing clones identified by restriction digest of mini prep DNA. Mini prep DNA from confirmed clones was then used to transform the heat-inducible AR58 strain of E. coli [SmithKline Beecham Laboratories]. Stocks of confirmed clones in AR58 were used to prepare induced cultures for expression analysis. As known to those skilled in the art, HB101 cells are not universally lambda cI857⁺. As a result, the P_{L} promoter will not be correctly regulated during culture growth in this strain. Additional transformations are performed in E. coli strain AR120, as AR120 has been characterized as being exclusively lambda cI⁺.

A plasmid containing a PCR-amplified fragment cloned into the XmaI-StuI sites of pOTSKF33 is illustrated in FIG. 2.

The remainder of the clones containing galK/FIPV S fusion proteins (SEQ ID NO: 20) were isolated using the following procedures. 2 μl of the designated PCR amplified reaction mix (approximately 500-1000 ng DNA) were digested with XmaI and StuI in a 30 μl volume of 50 mM Tris, pH 7.5, 10 mM MgCl₂, 10 mM BME, and 10 μg/ml BSA overnight at 37° C. One half of the digest reaction was loaded on 1% low-melting temperature agarose (Seakem) gels prepared and run in TBE. DNA fragments were isolated and eluted as described by T. Maniatis et al, cited above.

Briefly, DNA fragments were visualized after staining with ethidium bromide, excised from the gel with a scalpel and transferred to Eppendorf tubes. Gel slices were incubated 5 min at 65° C., vortexed, and 5 volumes of 20 ml Tris, pH 8.0, 1 mM EDTA were added. Samples were incubated an additional 2 minutes at 65° C. and were then extracted once with phenol and once with phenol:chloroform. The DNA was precipitated with 1/10 volume 3 M NaOAc and 2.5 volumes of cold 95% EtOH overnight at −20° C. Pelleted DNAs were resuspended and ligated overnight at 15° C. to pOTSKF33 plasmid DNA that was also digested with XmaI and StuI and phosphatased.

E. coli strain AR120 [SmithKline Beecham Laboratories] cells were transformed with ligation mixes and ampicillin-resistant transformants selected. Clones were screened for presence of insert by BamHI and PstI digestion of mini prep DNA. Mini prep DNA from insert-bearing clones was then used to transform AR58 cells. Confirmed clones in AR58 were used to prepare induced lysates for Western blot analysis.

FIG. 3 illustrates the PCR expression clone, AR58-3 (SEQ ID NO: 19 and 20, nucleotide and amino acid sequences, respectively). Sequencing was performed using a double stranded plasmid as a template. The construction consists of the following sequences: Nucleotides 1-168 originate from the pOTSKF33 at nucleotides 1880-2047, and encode 52 amino acids of galK. Nucleotides 169-181 of the AR58-3 encode an extraneous five amino acids. Nucleotides 182-573 of the clone originated from FIPV TS at nucleotides 356-734 and encode a 128 amino acid S gene region corresponding to published WSU 1146 strain amino acid #97-222. The total protein is 188 amino acids or about 22,500 kD using 120 as an average amino acid weight.

The predicted protein size agrees well with the band seen on both Coomassie gels and Western blots and contains functional XmaI and StuI sites. One additional amino acid results at the end of the FIPV protein due to the relegation of the StuI into pOTSKF33.

When compared to the WSU 1146 published strain, three base pair differences are apparent. The first difference lies at base number 312 in FIG. 3 (#480 of published sequence). WSU 1146 shows a “C”, while AR58-3 shows a “T”. No amino acid change results. The second difference lies at base number 349 of FIG. 3 (#517 of the published sequence). WSU 1146 shows an “A” and AR58-3 contains a “G”. An amino acid change from Threonine to Alanine results. The third difference lies at base number 399 of FIG. 3 (#567 of the published sequence). The published strain shows a “T” and AR58-3 contains a “C”. No amino acid change results. Additionally, a two amino acid insert (Tyr Ile) occurs in AR58-3 at amino acid numbers 84 and 85 in FIG. 3. These amino acids do not appear at a homologous position in the published WT WSU 1146 sequence.

### EXAMPLE 6

Western Blot of Induced Lysates

The bacterial clones containing the galK/FIPV S fusion genes are screened for expression by Western analysis.

Expression lysates of the fusion proteins in AR58 clones containing S sequences in pOTSKF33 were prepared as follows. 3 ml of LB+50 μg/ml ampicillin (amp) were inoculated with a bacterial colony using a sterile toothpick from a master plate and grown for 18 hours at 32° C. One ml of the overnight culture was then used to inoculate 50 ml of fresh LB+amp in 250 ml Erlenmeyer flasks. Cultures were grown at 32° C. in an air shaker until A₆₅₀=0.5-0.6. At this time, 1 ml T₀ samples were taken and cultures were induced by adding one-third volume of LB preheated to 65° C. Flasks were immediately transferred to a shaking water bath and incubated at 42° C. for 4 hours. A₆₅₀ values were again determined and 1.3 ml T₄ samples were taken.

Cells were pelleted and resuspended in sample buffer (0.1 M DTT, 2% SDS, 80 mM Tris, pH 6.8, 10% glycerol, 0.02% bromophenol blue) and stored at 20° C. Prior to electrophoresis, samples were denatured by boiling for 5 minutes at 100° C. Samples were vortexed and 15-20 μl loaded on 15% SDS-polyacrylamide gels as described by Laemmli, Nature, 227:680-685 (1970). Proteins were transferred to 0.2 um Schleicher+Schuell BAS/NC nitrocellulose for 30-45 minutes at 250 mA using a Milliblot apparatus or for 2 hours at 250 mA at 4° C. in Tris/glycine buffer using a Transblot apparatus (Bio-Rad).

Filters were blocked in 2% dry milk, 1% gelatin, TBS (20 mM Tris, pH 7.5, 500 mM NaCl) for 1 hour at room temperature, rinsed with TTBS (TBS+0.05% Tween-20) and incubated with rabbit polyclonal galK antisera, or the galK monoclonal antibody HIV env 41 AS1 [Beckman Instruments] in mouse ascites fluid, at a 1:1000 dilution in TTBS and 1% gelatin for 1 hour at room temperature. Filters were washed 3×10 min in TTBS and labelled with I¹²⁵ Protein A (1 uCi/10 ml) (Amersham) in TTBS and 1% gelatin for 1 hour at room temperature. Filters were washed as before, air-dried, and exposed to XAR film for various time periods at −70° C.

Table IV summarizes expression results of several FIPV S/pOTSKF33 AR58 clones. Bacterial lysates were prepared, run on SDS polyacrylamide gels, transferred to nitrocellulose, and analyzed by Western blot using both polyclonal and monoclonal galK antiserum as described above. The virus from which RNA was extracted for PCR amplification, the S amino acid region cloned in pOTSKF33, and the predicted size of the galK/S fusion protein are also shown.

TABLE IV

| | | | Fusion | |
|---|---|---|---|---|
| Clone | Virus | S Region* | Protein | Expression |
| 58-53 | TS DF2 | 1-105aa | 18.84 (kd) | ++ |
| 58-43 | TS DF2 | 1-223aa | 33 | ++ |
| 58-107 | FECV | 1-223aa | | ++ |
| 58-735 | WT DF2 | 1-223aa | | ++ |
| 58-3 | TS DF2 | 94-223aa | 21.86 | +++ |
| 58-399 | FECV | 94-223aa | | +++ |
| 58-465 | WT TN406 | 94-223aa | | +++ |
| 58-558 | WT DF2 | 94-223aa | | +++ |
| 58-565 | WT WSU 1146 | 94-223aa | | +++ |
| 58-494 | WT UCD-1 | 94-223aa | | +++ |
| 58-131 | WT DF2 | 94-223aa | | +++ |
| 58-885 | WT UCD-2 | 94-223aa | | +++ |
| 58-1542 | UCD-4 | 94-223aa | | +++ |
| 58-396 | FECV | 213-362aa | 24.24 | ++ |
| 58-437 | TS DF2 | 213-362aa | | ++ |
| 120-643-6 | WT DF2 | 213-362aa | | ++ |
| 58-462 | UCD-1 | 352-555aa | 30.72 | ++ |
| 58-470 | WSU | 352-555aa | | ++ |
| 58-515 | WT DF2 | 352-555aa | | ++ |
| 58-385 | WT DF2 | 352-748aa | 54 | ++ |
| 58-389 | TS DF2 | 352-748aa | | ++ |
| 58-391 | FECV | 352-748aa | | ++ |
| 58-438 | WT DF2 | 894-1040aa | 23.88 | ++ |
| 58-441 | TS DF2 | 894-1040aa | | ++ |
| 58-476 | FECV | 894-1040aa | | ++ |
| 58-426 | WT WSU 1146 | 894-1040aa | | ++ |
| 58-569 | WT UCD-1 | 894-1040aa | | ++ |
| 58-1133 | TS DF2 | 737-1040aa | 42.7 | +++ |
| 58-1138 | TS DF2 | 1029-1452aa | 57.1 | +++ |
| 120-896 | FECV | 94-748aa | | ++ |

*Amino acid numbers indicate sequences which correspond to published amino acid sequence of WT WSU 1146.

The results in Table IV show that the induced lysates of S/pOTSKF33 AR58 clones express fusion proteins of the predicted size as detected by polyclonal and monoclonal galK antiserum. Bands representing fusion proteins were not detected in uninduced lysates or control lysates of pOTSKF33 alone. Levels of expression are quantitated in Table IV as “+++” or “++”. The symbol “+++” indicates expression comparable to the level of expression produced by clone 58-3. Fusion proteins expressed to this high level are easily visualized on Coomassie stained polyacrylamide gels and may represent 5-10% of total cell protein.

The symbol “++” designates a level of expression less than that produced by 58-3 (SEQ ID NO: 20). In general, fusion proteins from these clones are not easily visualized in lysates stained with Coomassie Blues and may represent 1-2% of total cell protein.

### EXAMPLE 7

Induction of Large Cultures of Bacteria Expressing GalK/FIPV S Fusion Protein

Overnight stationary cultures of AR58 strain E. coli containing the fusion plasmid were used as inoculum for 500 mls of L Broth+100 μg/ml ampicillin. The cultures were incubated at 32° C. until OD₆₅₀ reached 0.5-0.6. One third culture volume of L Broth preheated to 65° C. was added and the cultures shifted to 42° C. for an additional 4 hours of growth. The bacteria were collected by centrifugation (3500, 10° C., 15 min) and resuspended in 100 ml H₂O. Lysozyme and EDTA (1% and 200 mM, respectively, 100 ml of each) were added to the cell pellet and cultures incubated on ice for 1 hour. The cultures were then sonicated in 50 ml aliquots for six minutes on ice (Branson sonifier) to completely disrupt the bacteria. Following sonication, thimerosal was added to a final concentration of 0.01-0.2% for 4-18 hours at 4° C. to inactivate the lysate. Aliquots of the inactivated material were used to inoculate LB plates with and without ampicillin. None of the cultures showed visible growth after 24 hours incubation.

### EXAMPLE 8

Solubilization of GalK/FIPV S Fusion Protein from Bacteria

Following induction of expression, the following purification protocol for isolation of pure galK/FIPV S fusion protein from bacterial lysates was performed.

Ten mls of the inactivated extract was centrifuged at 27,000×g for 30 min (JA20). The supernatant was discarded and the pellet resuspended by vortexing for 10 minutes in 10 mls of Buffer A plus 0.2% sodium deoxycholic acid and 1% Triton X-100. Buffer A contains 50 mM Tris-HCl, pH 8.5, 5 mM EDTA, 1 mM DTT, and 5% glycerol. The extract was centrifuged at 27,000×g for 30 minutes and again the resulting supernatant was discarded. The pellet was resuspended by vortexing 10 minutes in 10 mls of Buffer A containing Triton X-100 (1%) and 0.5 M KCl.

Following centrifugation (27,000×g, 30 minutes), the pellet was resuspended by vortexing 10 minutes in 2 mls of Buffer A containing 8 M urea. The solution was again centrifuged at 27,000×g for 30 minutes and the pellet discarded. The pH of the supernatant was adjusted by stepwise addition of 10 mM Na phosphate buffer, pH 7.4, until the solution reached a volume of 20 mls (final urea concentration, 0.8M).

### EXAMPLE 9

Purification of Anti-Galactokinase Monoclonal Antibodies

Ascites fluid containing anti-galactokinase mAbs was produced in mice against the first 52 amino acids of galK, e.g., HIV env 41 AS1 [Beckman Instruments].

The BCA Protein Assay Kit [Pierce Chemical Co.] which consists of a bicinchoninic acid solution and a copper sulfate solution, was used according to manufacturer's instructions to determine the concentration of protein in the fluid. Copper 2+ ions in the assay are converted to copper 1+ in the presence of protein. Copper 1+ ions are then chelated to BCA molecules, resulting in a colorimetric change. The higher the protein concentration, the deeper the color. Protein concentrations are determined from absorbance measurements at 562 nm.

90 mg of total protein was added to sterile phosphate buffered saline (PBS), pH 7.4. The material was stirred on ice while ammonium sulfate was added to a final concentration of 45%. After 2 hours on ice, the precipitate was collected by centrifugation at 3000×g for 30 minutes at 4° C. The supernatant was discarded and the pellet resuspended in PBS with gentle vortexing. Again while slowly stirring on ice, saturated ammonium sulfate was added to 40%. After 1 hour, the precipitate was collected by centrifugation as previously described.

The supernatant was discarded and the pellet resuspended in PBS by vortexing. The mAb mixture was added to Spectrapor membrane tubing (M.W. cutoff 12-14000) [Fisher Scientific] and dialyzed against 4 changes of 4 liters of PBS, pH 7.4. The post-dialysate contained 19.5 mg of total protein.

### EXAMPLE 10

Affinity Purification of GalK/FIPV S Fusion Proteins

The anti-galactokinase mAbs were coupled to column matrix using the Immunopure™ Ag/Ab Immobilization Kit [Pierce Chemical Co]. Ten mg of anti-galactokinase Abs were immobilized on Aminolink™ (agarose) as described by the manufacturer. Columns were stored at 4° C.

Both the column and the pre-column solution containing fusion protein were brought to room temperature. The column was equilibrated with 16 mls of 10 mM Na phosphate buffer, pH 7.4. The pre-column solution was applied to the column in 4 aliquots of 5 ml each. The total pre-column eluate was re-added to column three times.

The column was then washed with 6 mls of 0.8 M urea followed by washing with 10 mM phosphate buffer, pH 7.4 (16 mls). The bound fusion protein was eluted by the addition of 5 mls of 0.1 M glycine, pH 2.8. Five 1 ml fractions were collected and neutralized with 50 μl of 1 M Tris-HCl, pH 9.5. The fractions were stored at 4° C. until needed. The column was re-equilibrated with 10 mM Na phosphate buffer, pH 7.4.

Galactokinase ELISA affinity column eluted fractions were diluted 1:100 in 10 mM borate buffer, pH 9.6, and 100 μl aliquots added to each well of 96 well plates (Nunc Inmuno plates). The plates were incubated overnight at 4° C., then brought to room temperature and washed once with PBS (pH 7.4) containing 0.05% Tween-20 (PBS-Tween). Blocking agent (PBS+1% polyvinyl alcohol, PVA) was added in 200 μl aliquots to each well for 30 min at 37° C. The plate was washed once with PBS-Tween and then 100 μl of the mouse anti-galactokinase, mAb (1:1000) in PBS+1% PVA added to each well.

After 1 hour at 37° C., the plates were washed once with PBS-Tween. Goat anti-mouse IgG peroxidase labelled conjugate (Kirkegaard and Perry) was diluted 1:1000 in PBS+1% PVA and aliquots of 100 μl added to each well. The plates were incubated for 1 hour at 37° C. and then washed one time with PBS-Tween. Aliquots of 100 μl of the ABTS peroxidase substrate system (Kirkegaard and Perry) were added to each well and after 10 minutes of incubation at room temperature, the intensity of chromogenic-reaction was measured at 405 nm on a Molecular Devices Vmax plate reader.

### EXAMPLE 11

Western Analysis Using Cat Sera

Affinity column eluted fractions of fusion proteins were denatured with Laemmli sample buffer and electrophoresed on preparative 10% SDS-polyacrylamide gels. Following electrophoresis, the proteins were transferred to nitrocellulose according to the procedure of Towbin et al, Proc. Natl. Acad. Sci. USA, 76:4350-4354. The nitrocellulose was incubated overnight at room temperature in blocking solution containing 50 mM Tris, pH 7.4, 150 mM NaCl and 5% non-fat dried milk (Buffer A). Following blocking, the nitrocellulose was sliced into 5 mm strips and placed into individual incubation chambers.

Each strip was incubated for 1 hour at room temperature with unique cat sera diluted 1:30 in 3 ml of 50 mM Tris, pH 7.4, 150 mM NaCl, 0.2% Triton X-100 and 5% non-fat dried milk (Buffer B). The strips were then washed for 15 minutes with Buffer A followed by one Buffer B wash. Goat anti-cat IgG phosphatase labelled conjugate (Kirkegaard and Perry), diluted 1:1000 in Buffer A, was added to each chamber for 1 hour at room temperature. The nitrocellulose strips were then washed successively with Buffer B. Buffer A and then twice with Buffer C (20 mM Tris, pH 7.4, 500 mM NaCl, 5% non-fat dried milk).

BCIP/NBT phosphatase substrate system [Kirkegaard and Perry] was added to each strip; the reaction was stopped by decanting the substrate and washing with H₂O after 30 minutes at room temperature.

Western blots were performed to determine the binding affinity of purified fusion proteins to sera from cats challenged either with WT DF2 FIPV or WT WSU 1146. For cats challenged with WT DF2 FIPV, the following was performed: three weeks post second TS-FIPV vaccinated serum and four weeks post WT DF2 FIPV challenged serum, both from non-symptomatic cat #IR03; three weeks post second TS-FIPV vaccinated serum and four weeks post WT DF2 FIPV challenged serum, both from symptomatic cat #JI1; non-vaccinated pre-challenge serum and four weeks post WT DF2 FIPV challenged serum, both from non-symptomatic cat #G26; non-vaccinated pre-challenge serum and four weeks post WT DF2 FIPV challenged serum, both from symptomatic cat #IRV5; (mAb) anti-galactokinase monoclonal antibody; (J736) serum from rabbit which was immunized with S peptide conjugate ovalbumin-glutaraldehyde-137-151 amino acid fragment; and (J739) serum from rabbit which was immunized with S peptide conjugate ovalbumin-glutaraldehyde-150-180 amino acid fragment.

Only post FIPV challenged sera from symptomatic cats recognized the 22 kD FIPV galK/S fusion protein expressed by recombinant 58-3 (SEQ ID NOS: 19 and 20). The anti-galactokinase mAb and rabbit sera served as positive controls.

For cats challenged with WT WSU 1146, the following was performed: three weeks post second TS-FIPV vaccinated serum, and four weeks post WT WSU 1146 FIPV challenged serum, both from non-symptomatic cat #EU6; three weeks post second TS-FIPV vaccinated serum and four weeks post WT WSU 1146 FIPV challenged serum, both from symptomatic cat #FW3; non-vaccinated pre-challenge serum,and four weeks post WT WSU 1146 FIPV challenged serum, both from non-symptomatic cat #FV2; non-vaccinated pre-challenge serum and four weeks post WT WSU 1146 FIPV challenged serum, both from symptomatic cat #PB2; (mAb) anti-galactokinase monoclonal antibody; (J736) serum from rabbit which was immunized with S peptide conjugate ovalbumin-glutaraldehyde-137aa-151aa; (J739) serum from rabbit which was immunized with S peptide conjugate ovalbumin-glutaraldehyde-150aa-180aa; and a Control which received only second conjugated goat-anti-cat IgG-Phosphatase antibody. Only post FIPV challenged sera from symptomatic cats recognized the 22 kd FIPV galK/S fusion protein expressed by recombinant 58-3 (SEQ ID NOS: 19 and 20). The anti-galactokinase mAb and rabbit sera served as positive controls.

### EXAMPLE 12

Partial Sequences of Other Strains

In the course of the present invention, the following DNA and amino acid sequences of the complete S gene WT DF2 FIPV (SEQ ID NOS: 21 and 22, respectively), a fragment of the S gene of DF2-HP (SEQ ID NOS: 23 and 24, respectively), the complete S gene TS (SEQ ID NOS: 25 and 26, respectively), a fragment of the S gene of TS-BP (SEQ ID NOS: 27 and 28, respectively), a fragment of the S gene of the WT TN406 (SEQ ID NOS: 29 and 30, respectively), a fragment of the S gene of UCD-2. (SEQ ID NOS: 53 and 54, respectively) and the complete S gene of the FECV (SEQ ID NOS: 31 and 32, respectively) have been obtained by procedures substantially similar to those described in Example 1 for WT WSU 1146.

FIG. 4 provides the sequences of the complete S gene of the WT FIPV DF2 virus (SEQ ID NOS: 21 and 22) and a fragment of the S gene of the FIPV DF2-HP virus (SEQ ID NOS: 23 and 24). The bold print indicates the places where the sequence of DF2-HP differs from WT DF2. Nucleotide changes in DF2-HP from WT DF2 are indicated above the WT DF2 sequence with an asterisk and amino acid differences are indicated below the WT DF2 sequence with an asterisk.

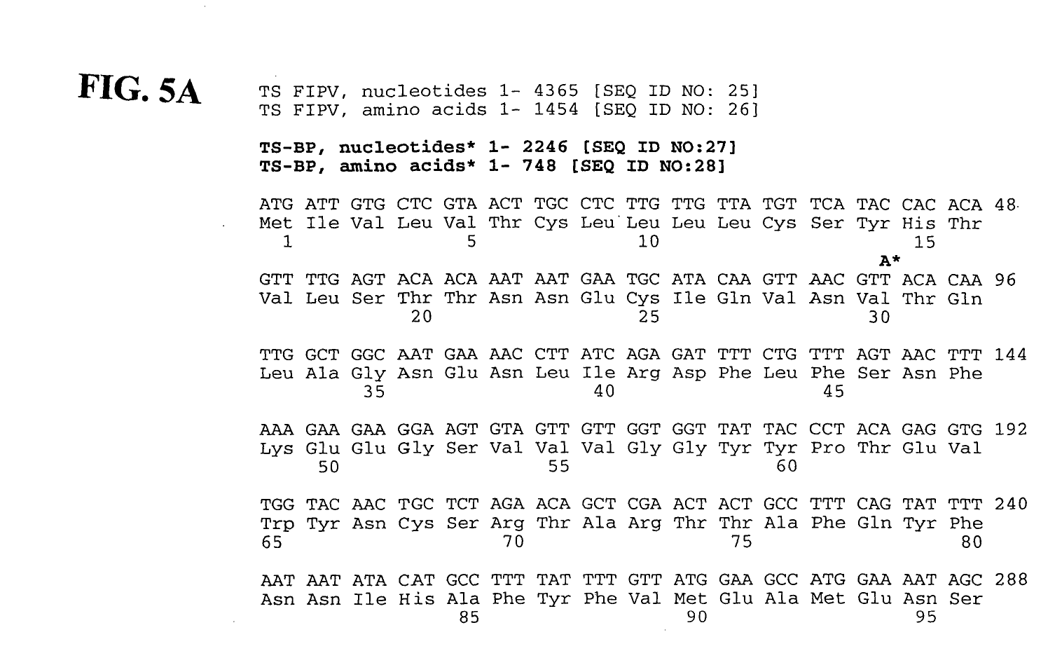

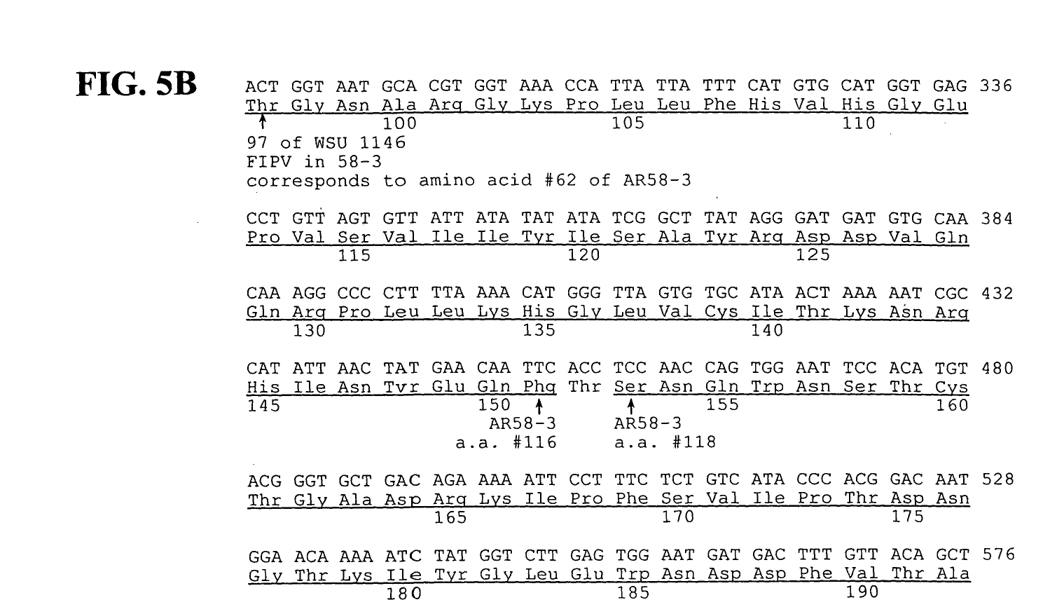

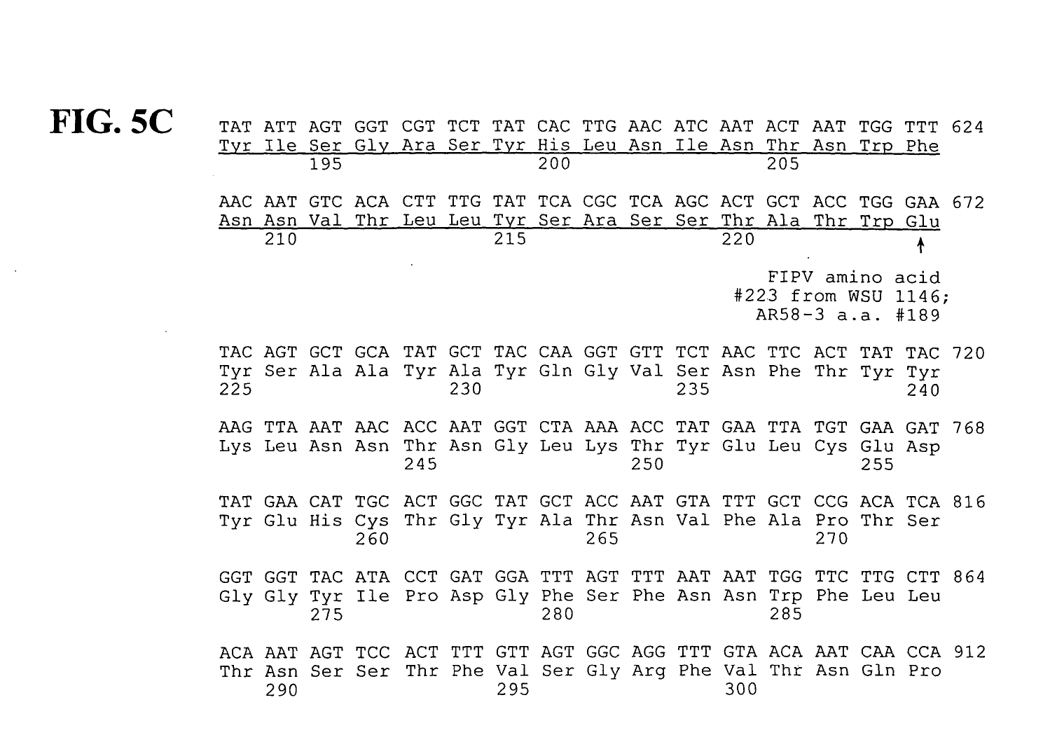

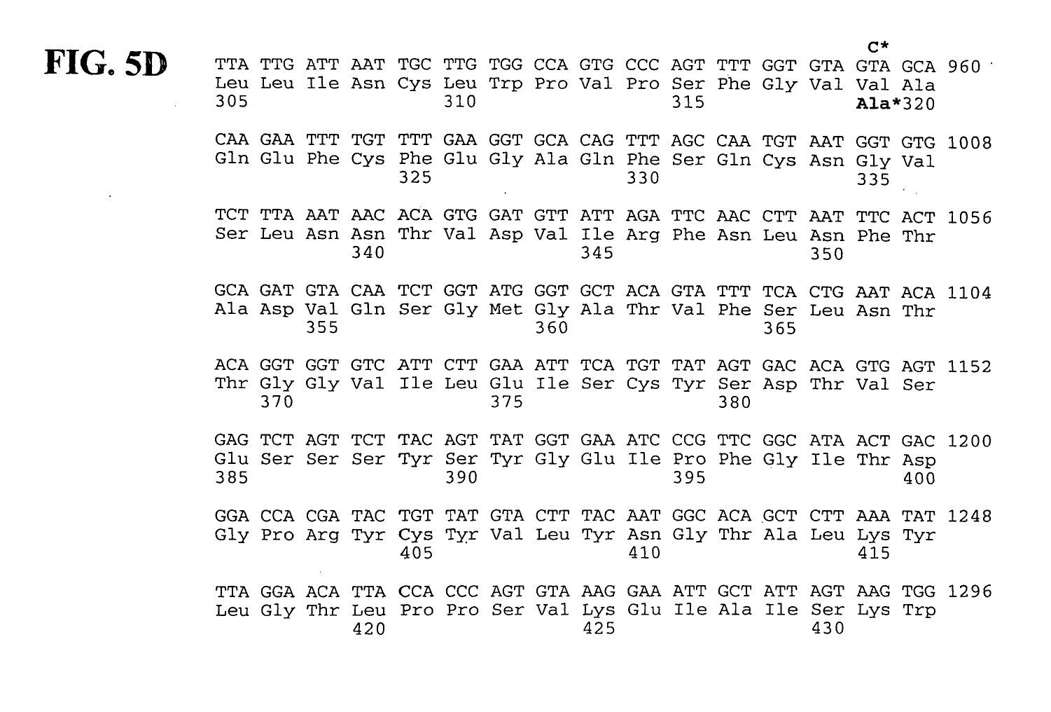

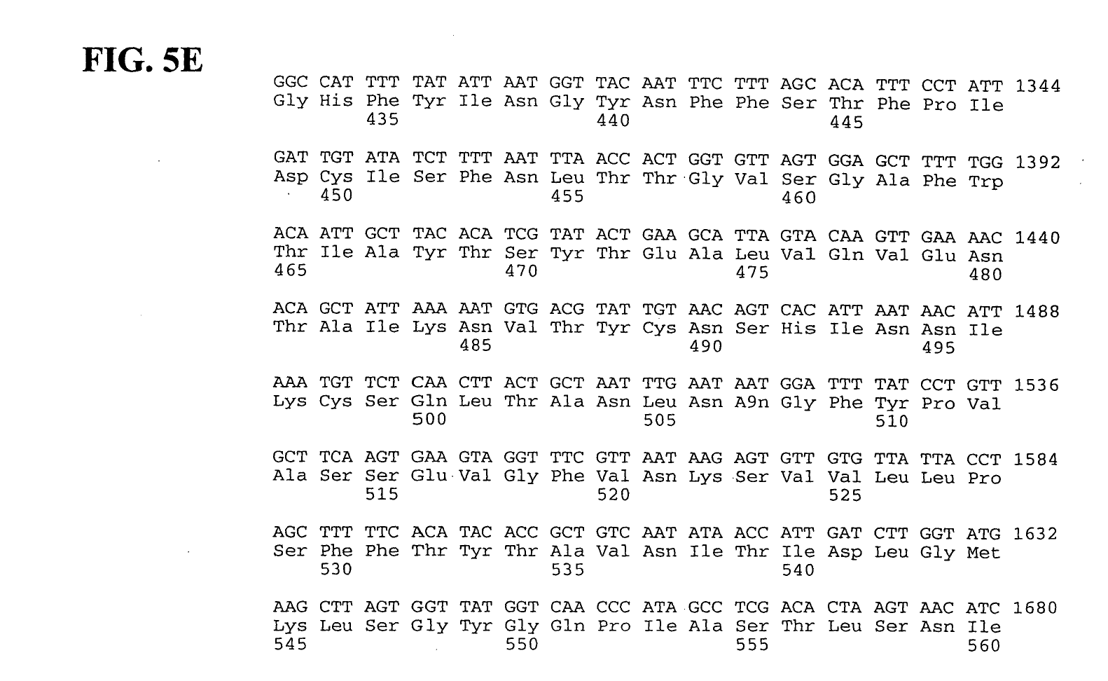

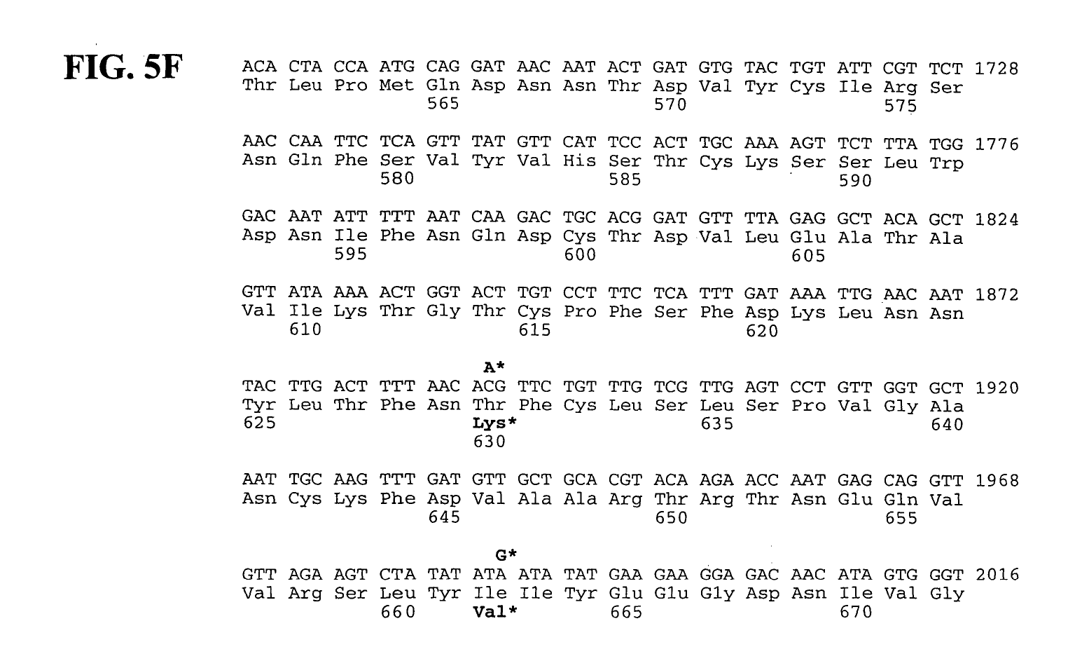

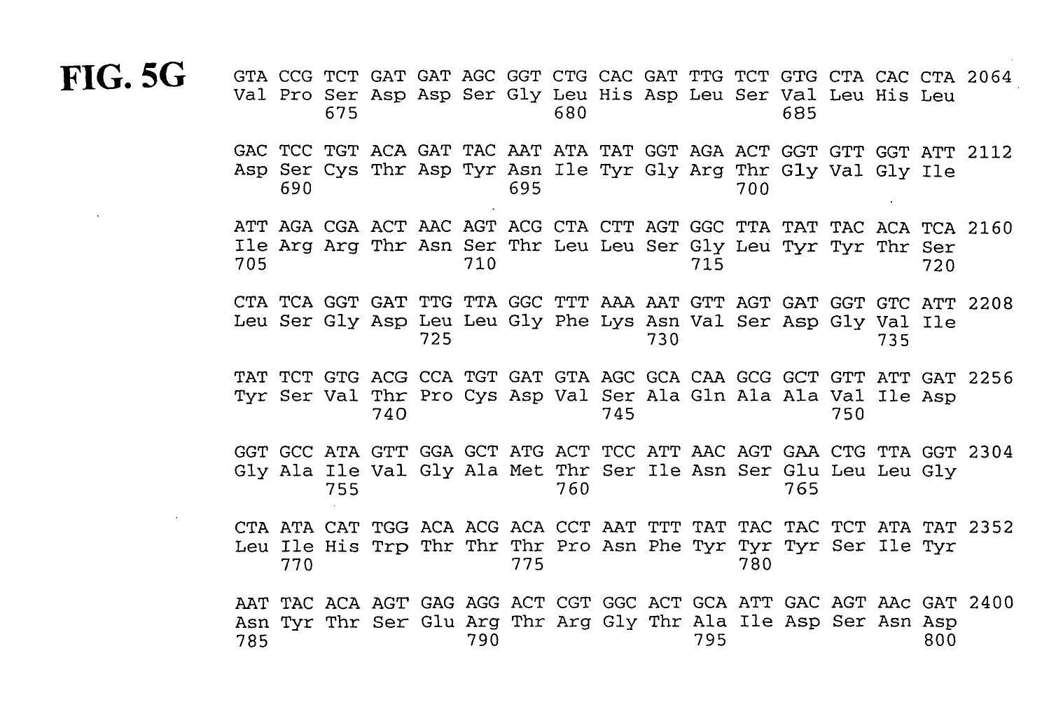

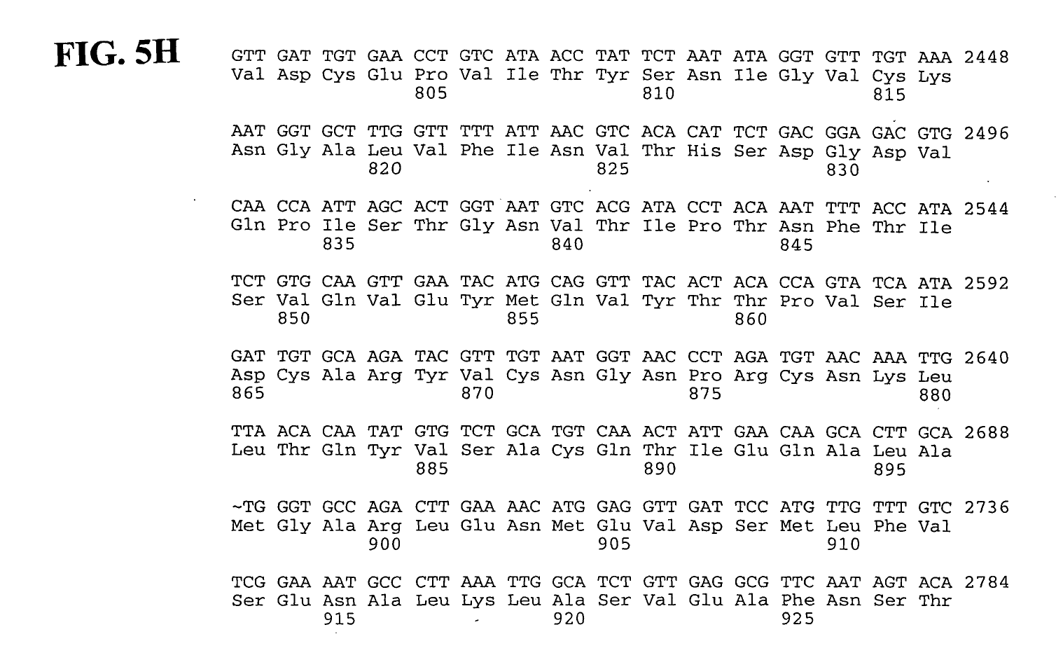

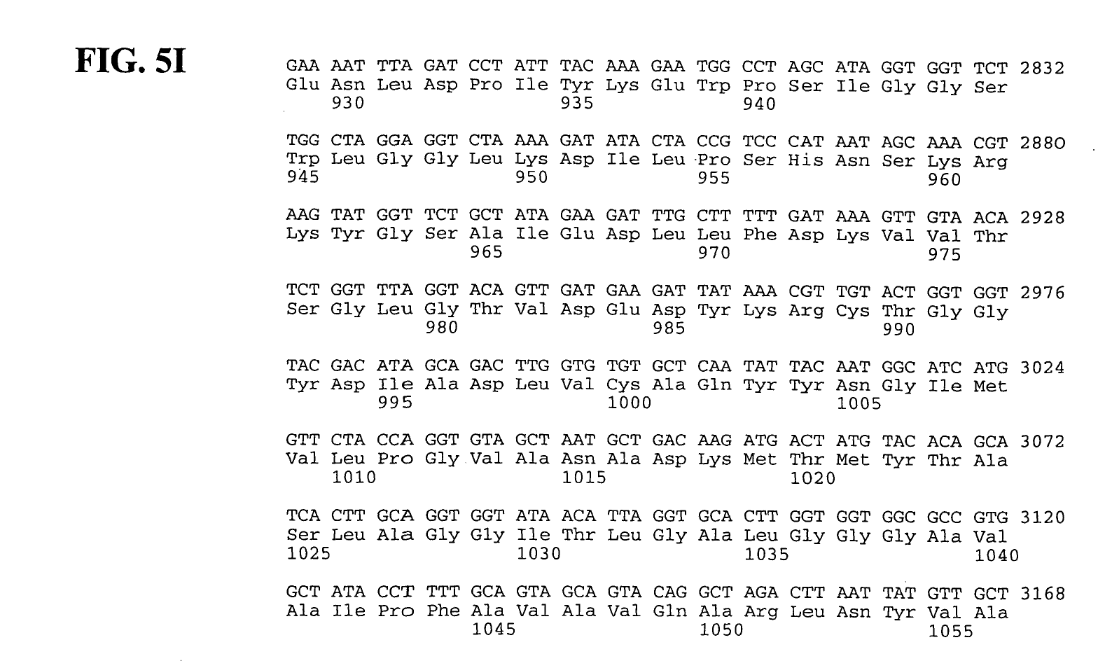

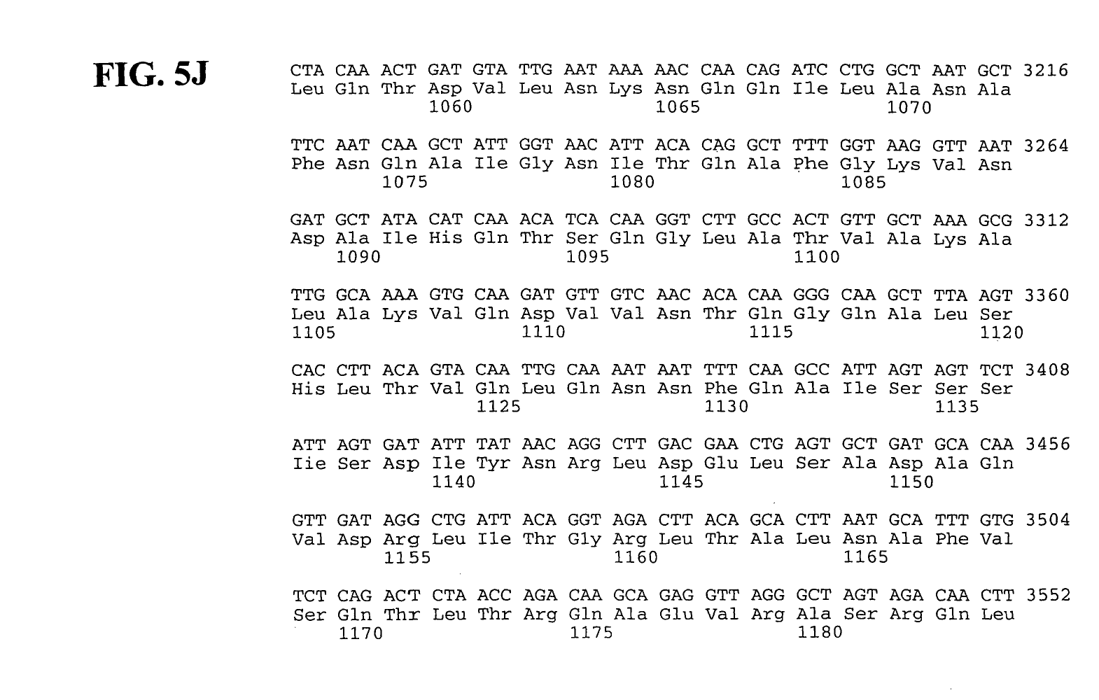

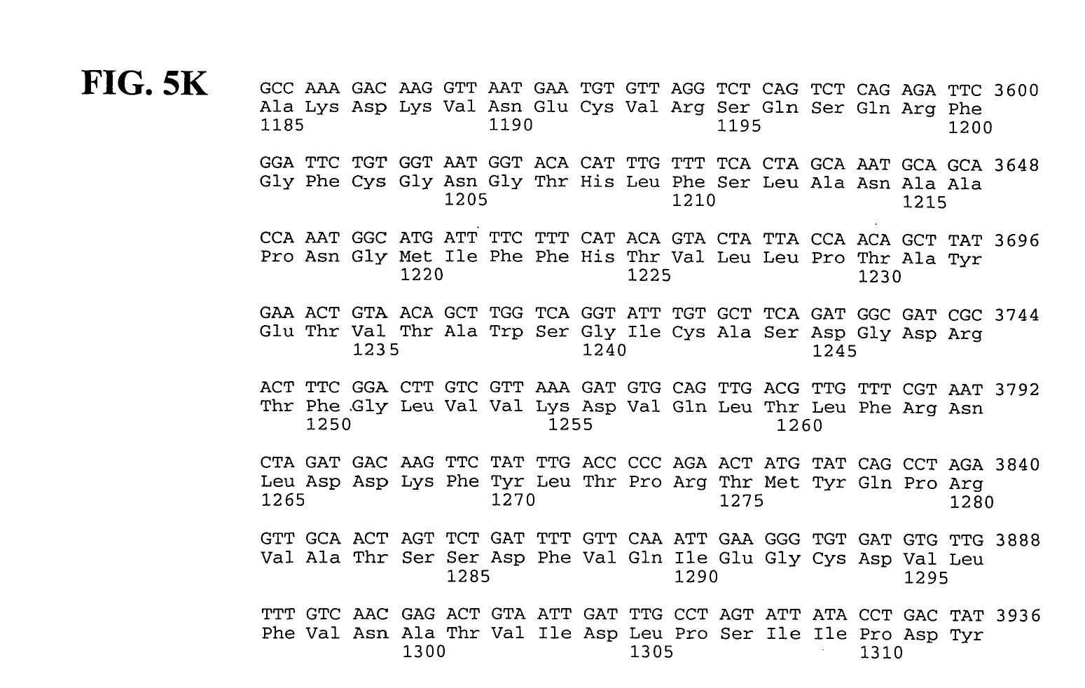

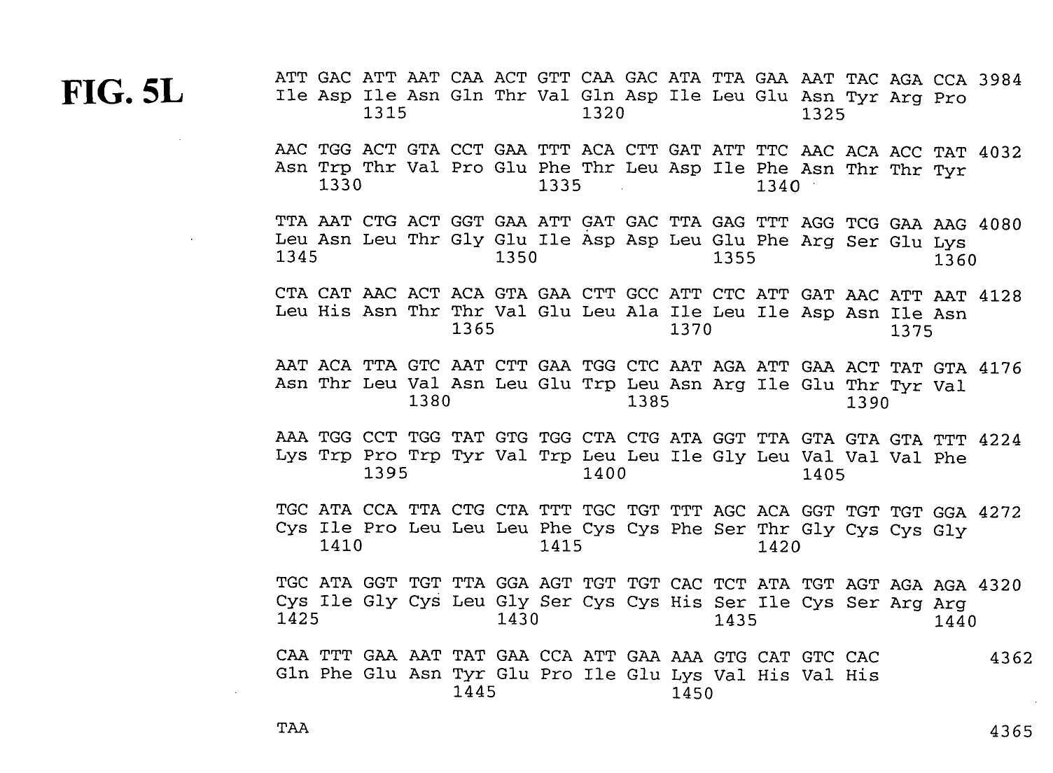

FIG. 5 provides the sequences of the complete S gene of the TS FIPV (SEQ ID NOS: 25 and 26) and a fragment of the S gene of the TS-BP (SEQ ID NOS: 27 and 28) from amino acids 1-748, which each include a sequence homologous to the AR58-3 S-derived peptide (SEQ ID NO: 20). Nucleotide differences in the TS-BP sequence from TS are indicated in bold type above the TS sequence with an asterisk and amino acid differences are similarly indicated below the TS sequence.

Certain areas of homology between AR58-3, as illustrated in FIG. 3 (SEQ ID NO: 20), and the sequences of TS FIPV (SEQ ID NOS: 25 and 26), WT DF2 FIPV (SEQ ID NOS: 21 and 22) are indicated by underlining in FIGS. 4 and 5 below.

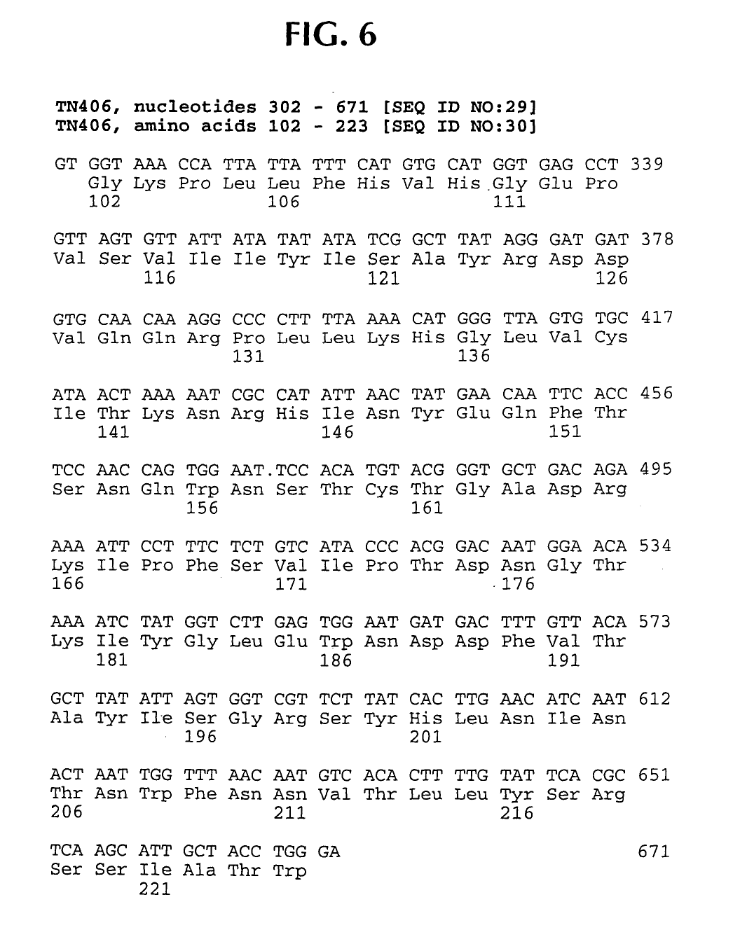

FIG. 6 provides the sequences of the WT TN406 FIPV from amino acid 102-223 (SEQ ID NO: 29 and 30).

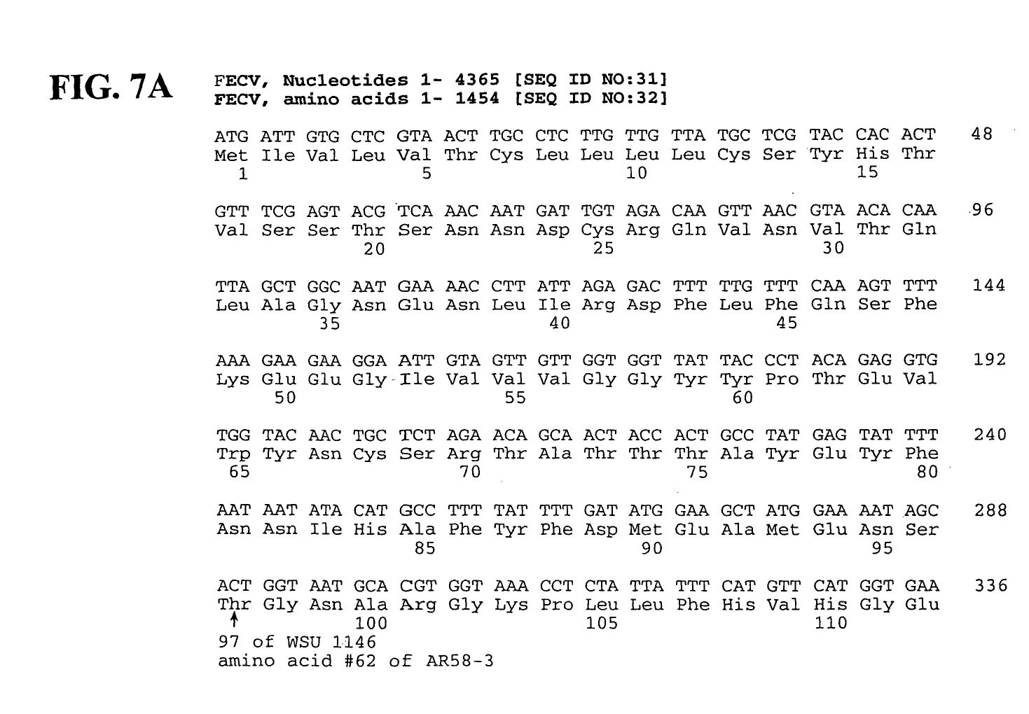

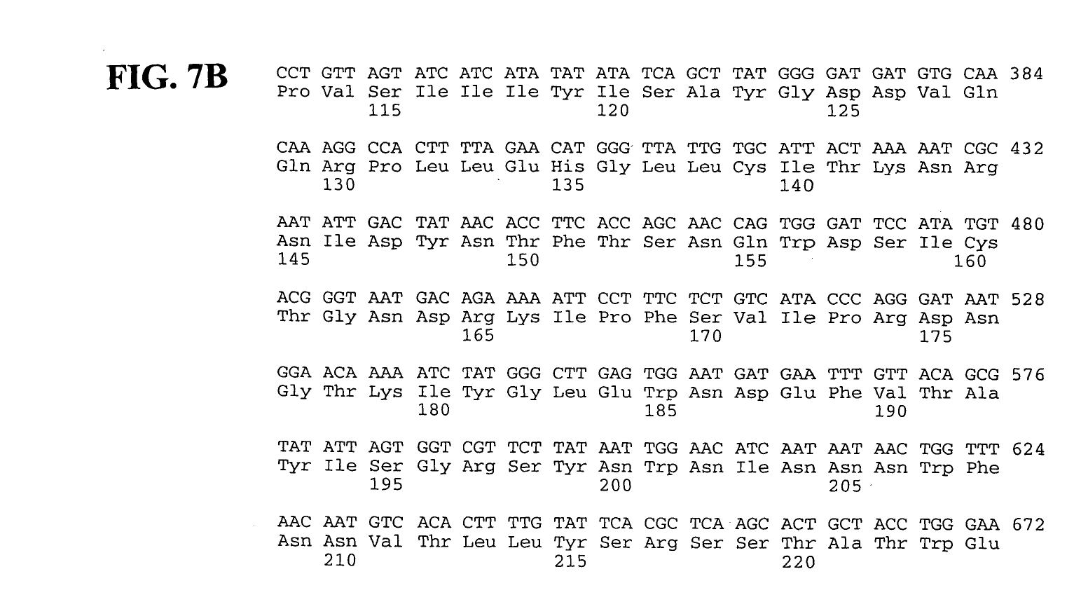

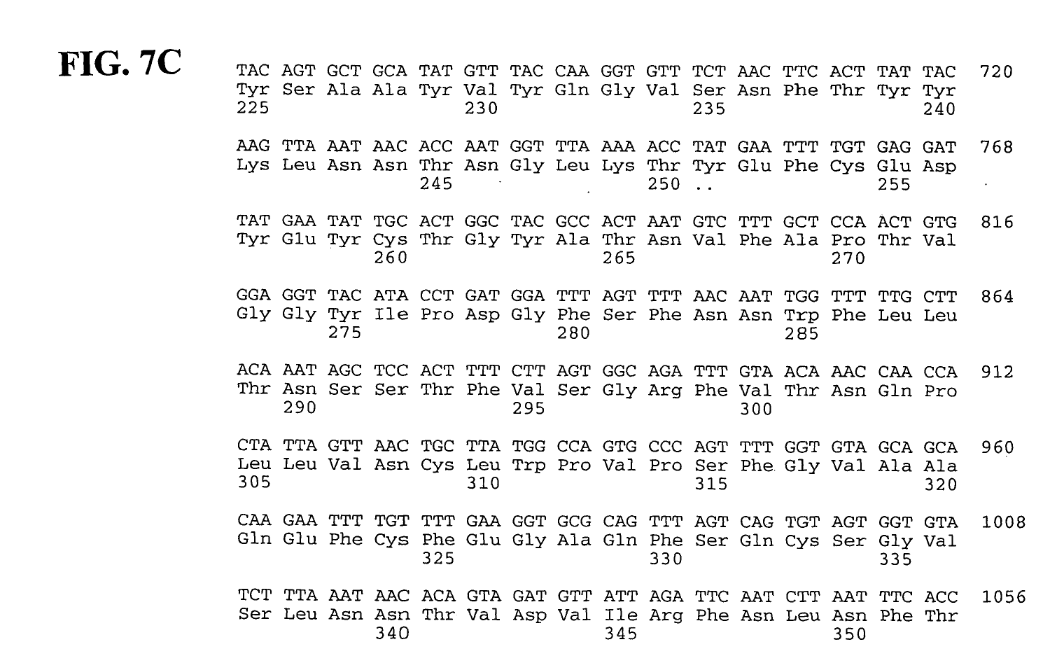

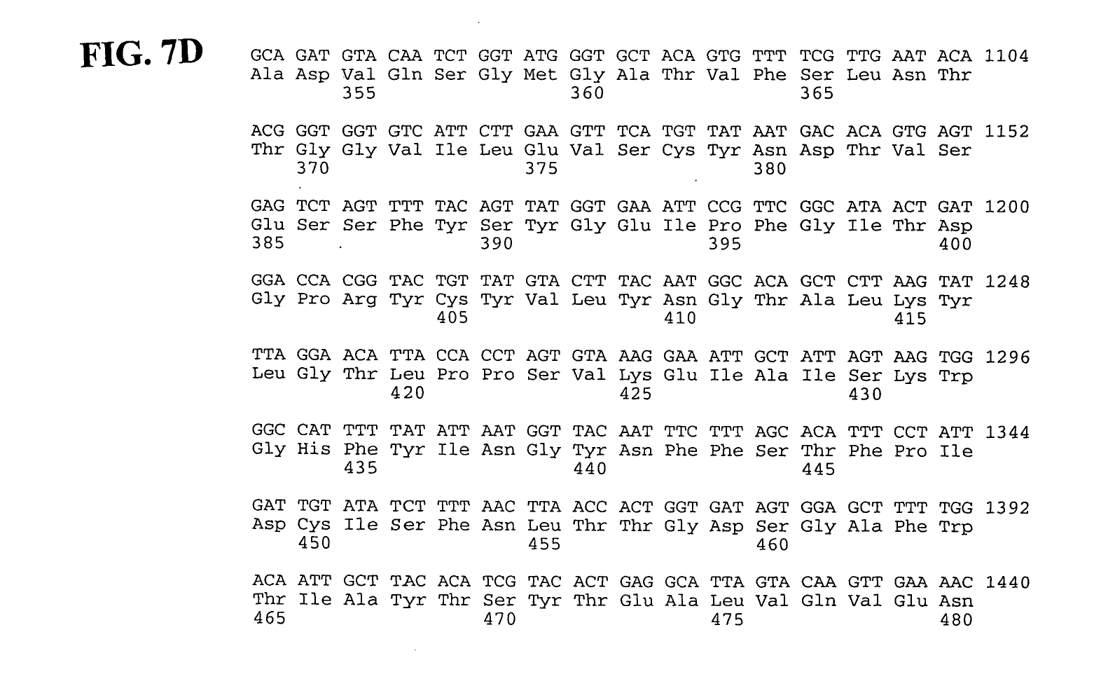

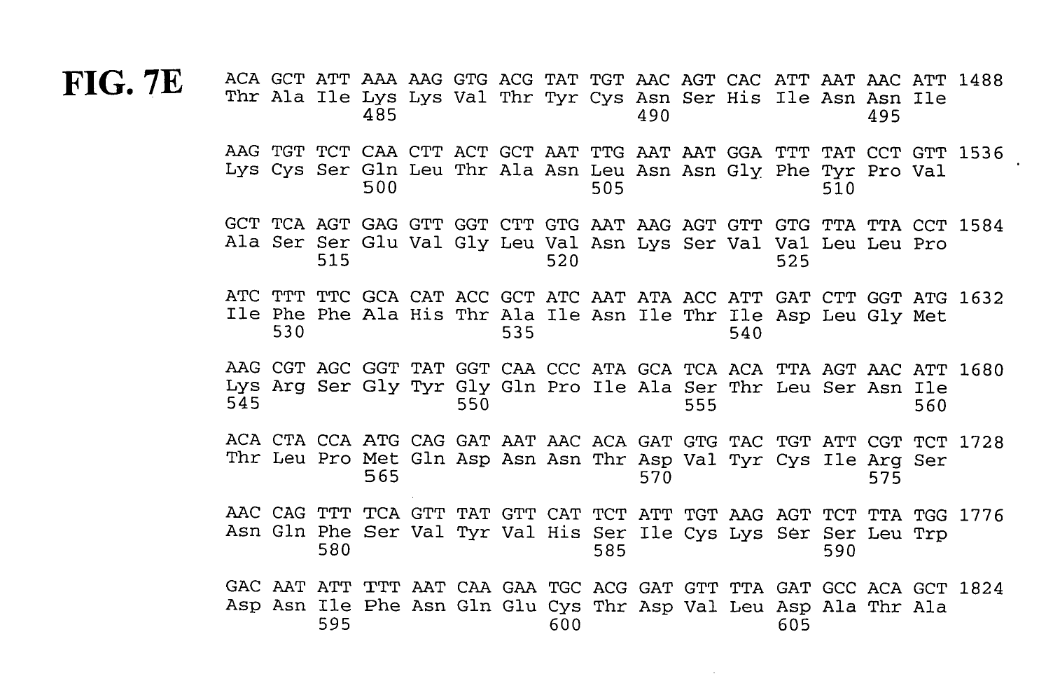

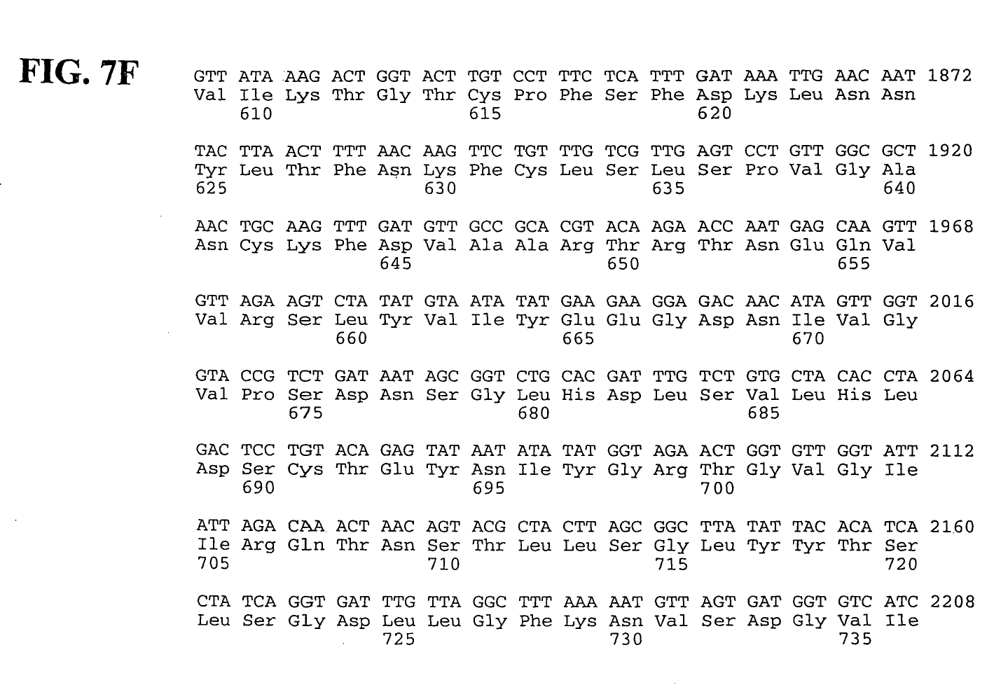

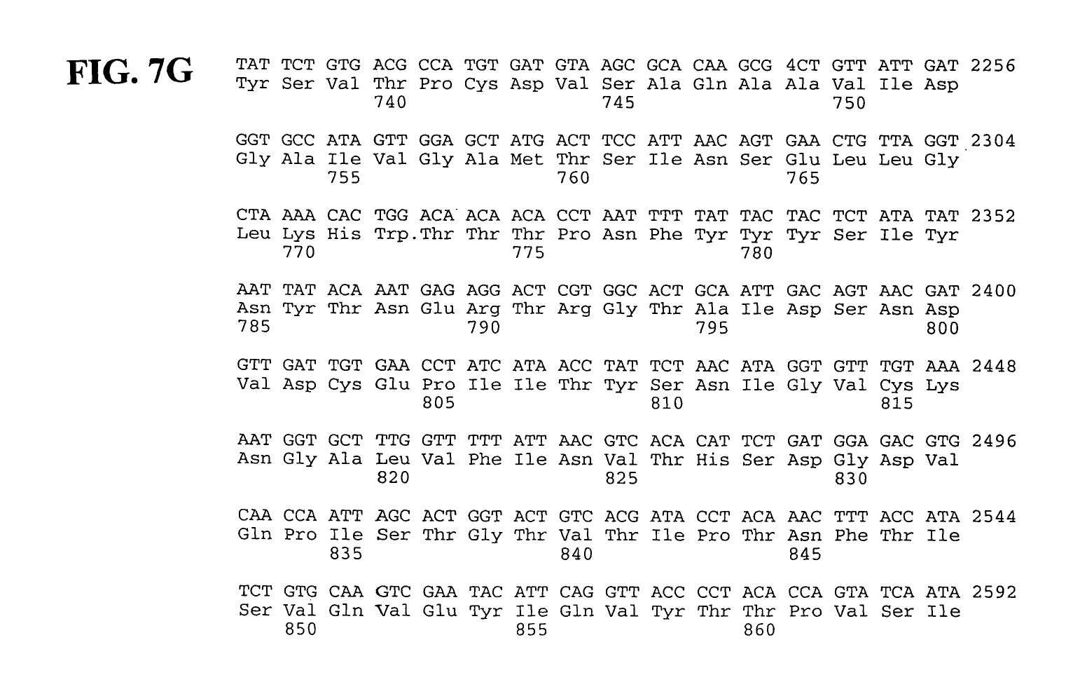

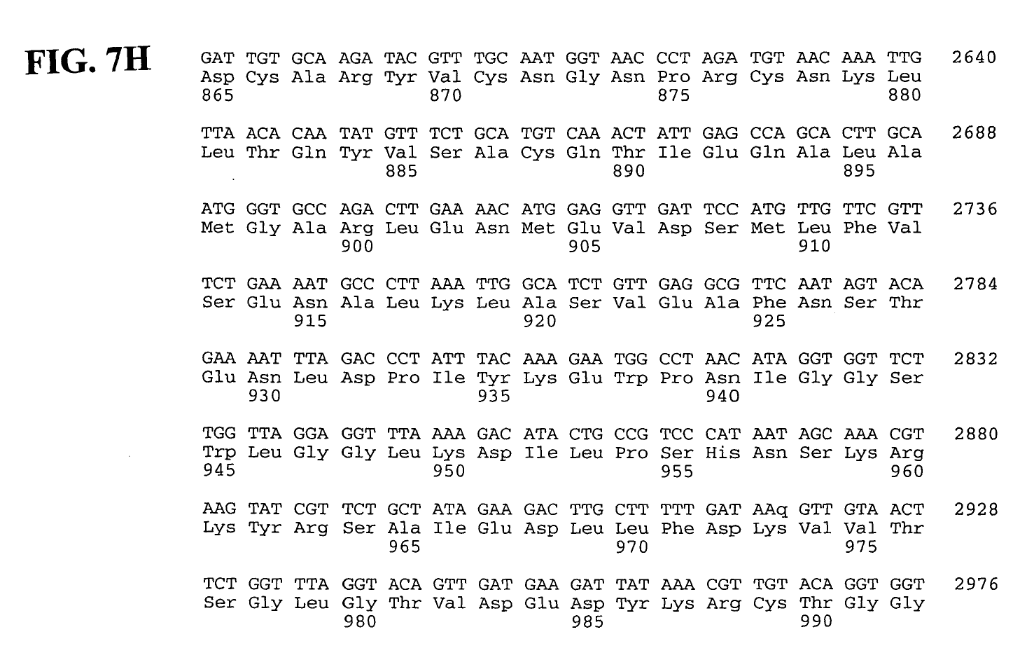

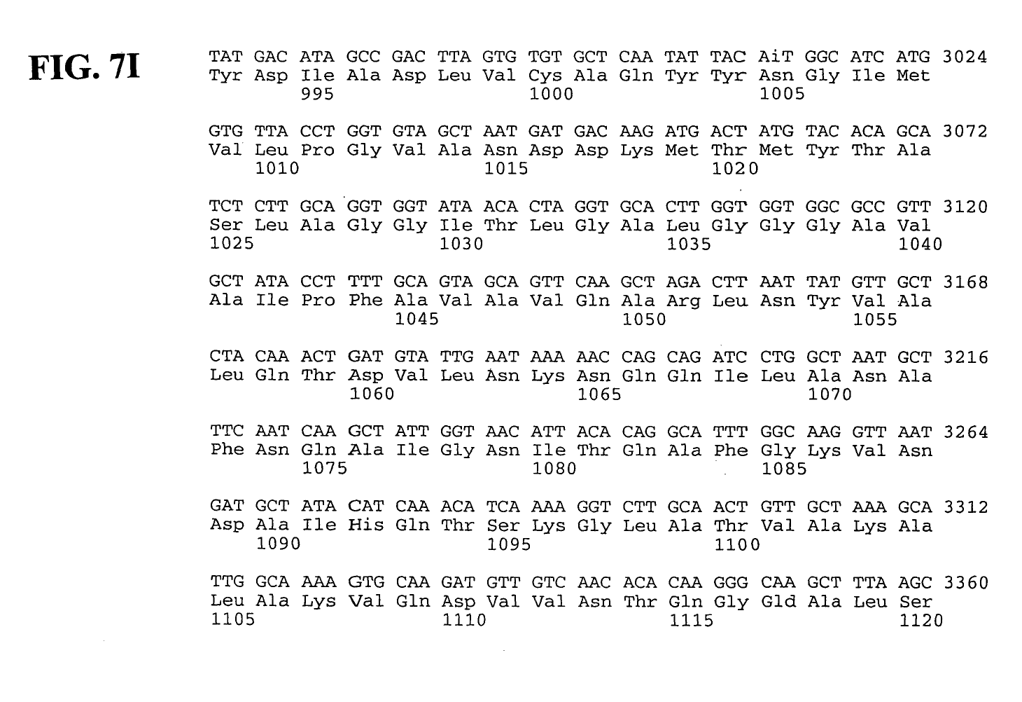

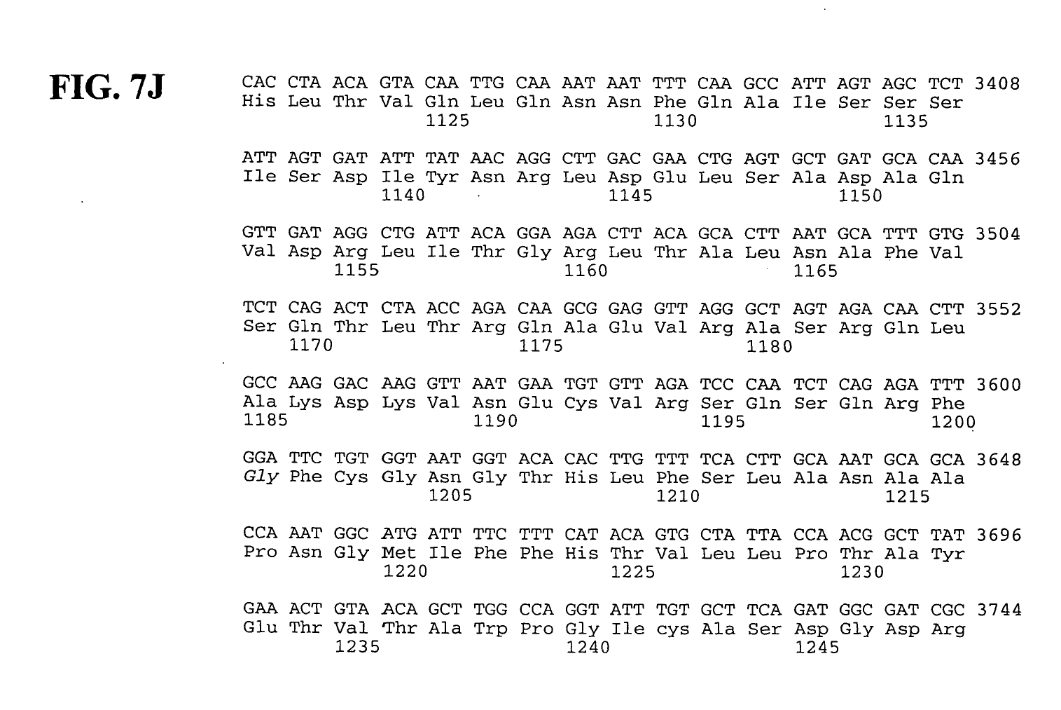

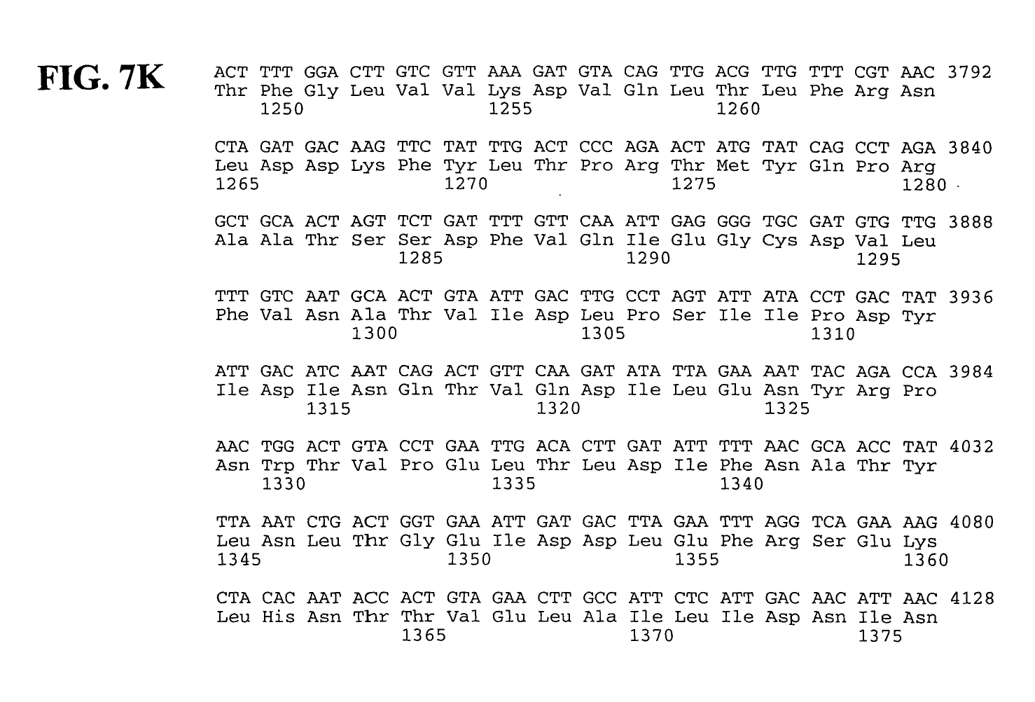

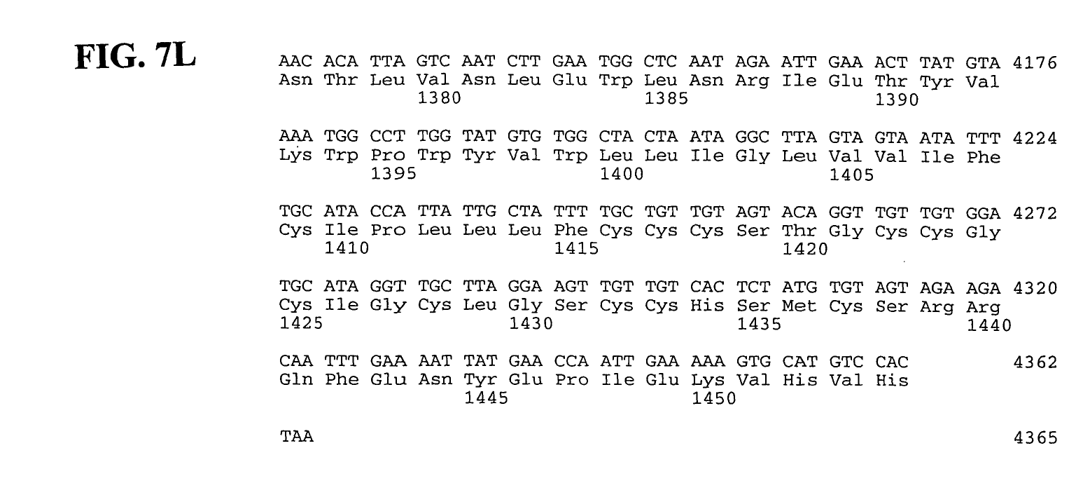

FIG. 7 provides the sequences of the S gene of the FECV virus from amino acid 1-1452 (SEQ ID NOS: 31 and 32).

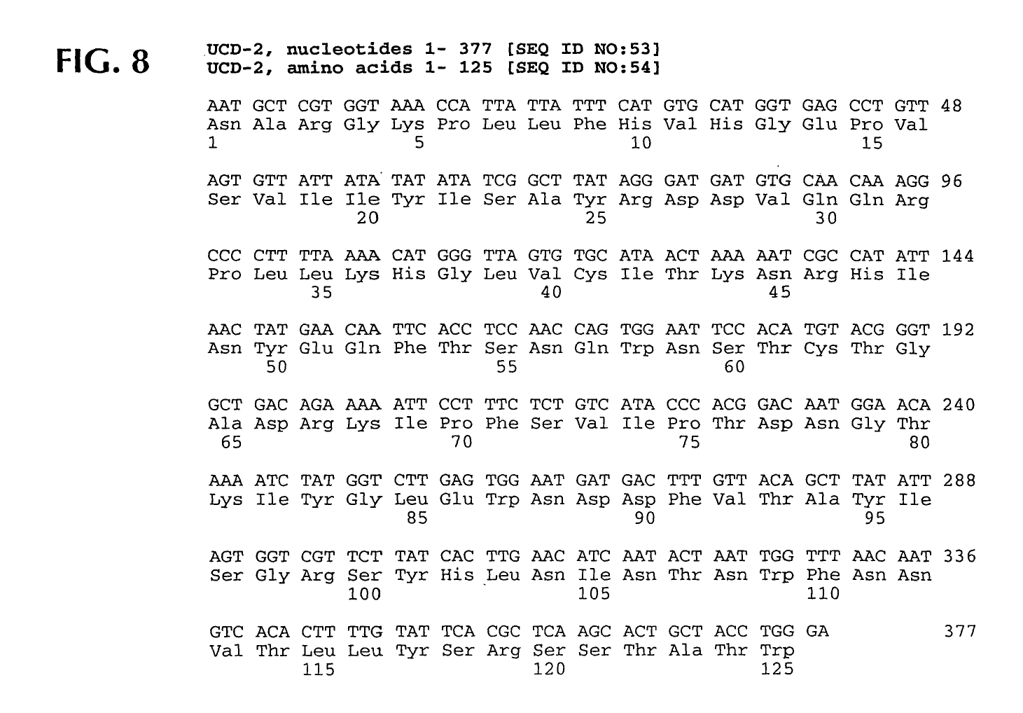

FIG. 8 provides the sequences of the S gene of the UCD-2 virus from amino acid 1-125 (SEQ ID NO: 53 and 54).

Differences between the nucleotide and amino acid sequences of FIPV, strains WT WSU 1146, WT DF2, DF2-HP, TS, TS-BP, WT TN406, FECV, UCD-2 and the Consensus Sequence, which extends from nucleotides 1-2246 (encoding amino acid 1-748) of the S gene are as follows, with the Consensus Sequence illustrated in FIG. 9 (SEQ ID NOS: 33 and 34) serving as the reference. No consensus sequence has been obtained for that portion of the gene beyond amino acid 748 (base pair 2246). Therefore, for the strains for which the genes have been sequenced beyond this point, reference is made to the published WT WSU 1146 sequence.

WT WSU 1146 differs from the Consensus Sequence (SEQ ID NO: 33) by the following nucleotide changes: C at 849; A at 2029; G at 1346 and deletions: 351-356. WT WSU 1146 contains the following amino acid changes: Gly at 449 and Asn at 677 and deletions: 119 and 120.

WT DF2 (SEQ ID NO: 21) differs from the Consensus Sequence (SEQ ID NO: 33) by the following nucleotide changes: A at 216, A at 218, C at 849, G at 1346, C at 1370, C at 1597, C at 1751, A at 2029. WT DF2 (SEQ ID NO: 22) contains the following amino acid changes: Gln at 73; Gly at 449, Ala at 459; His at 533; Pro at 584, and Asn at 677.

In addition, WT DF2 (SEQ ID NO: 21) differs from the published WT WSU 1146 sequence by the following nucleotide changes (the corresponding WT WSU 1146 numbers follow in parentheses): C at 2541 (T at 2601); C at 4121 (A at 4185); C at 4210 (T at 4273); T at 4330 (A at 4394.). WT DF2 (SEQ ID NO: 22) differs from the published WT WSU 1146 sequence by the following amino acid differences: Thr at 1374 (Asn at 1372) and Tyr at 1444 (Asn at 1442).

DF2-HP (SEQ ID NO: 23) differs from the Consensus Sequence (SEQ ID NO: 33) by the following nucleotide changes: G at 400; C at 1083; T at 849; G at 1346; C at 1791 and G at 2029. DF2-HP (SEQ ID NO: 24) contains the following amino acid changes: Glu at 134; Gly at 449 and Asp at 677.

TS (SEQ ID NO: 25) differs from the Consensus Sequence (SEQ ID NO: 33) by the following nucleotide changes: T at 90; T at 849; T at 956; A at 1346; C at 1889; A at 1984; and G at 2029. TS (SEQ ID NO: 26) contains the following amino acid changes: Val at 319; Thr at 630; Ile at 662; Asp at 449; and Asp at 677.

In addition, TS [SEQ ID NO: 25] differs from the published WT WSU 1146 sequence by the following nucleotide changes: T at 2309 (C at 2372); C at 2541 (T at 2604); A at 4024 (G at 4087) and G at 4074 (A at 4137). TS [SEQ ID NO: 26] differs from the amino acid sequence of WT WSU 1146 by the following amino acid changes: Ile at 770 (Thr at 768) and Thr at 1342 (Ala at 1340).

TS-BP (SEQ ID NO: 27) differs from the Consensus Sequence (SEQ ID NO: 33) by the following nucleotide changes: T at 849; A at 1346; G at 2029. TS-BP (SEQ ID NO: 28) contains the following amino acid inserts: Asp at 449 and Asp at 677.

WT TN406 (SEQ ID NO: 29) differs from the Consensus Sequence (SEQ ID NO: 33) by the following nucleotide changes: T at 659. WT TN406 (SEQ ID NO: 30) contains an amino acid change to Ile at position 220.

FECV (SEQ ID NO: 31) differs from the Consensus Sequence (SEQ ID NO: 33) by the following nucleotide changes: C at 36, T at 48, C at 53, G at 60, T at 61, C at 66, T at 72, T at 75, G at 77, A at 99, T at 120, C at 126, T at 130, T at 141, T at 158, A at 230, G at 232, A at 266, T at 276, T at 312, C at 313, T at 327, A at 336, A at 346, C at 348, C at 351, A at 360, G at 370, A at 393, G at 400, T at 412, T at 420, A at 433, G at 439, A at 445, C at 447, A at 448, C at 449, C at 450, A at 457, G at 458, G at 469, T at 476, A at 487, A at 488, G at 521, T at 525, G at 546, A at 564, G at 576, A at 598, T at 600, G at 602, A at 614, C at 618, T at 689, T at 742, T at 759, G at 765, T at 775, C at 789, C at 792, T at 795, C at 801, A at 810, T at 813, G at 814, T at 815, G at 816, A at 819, C at 849, T at 858, C at 873, A at 894, C at 906, C at 913, A at 918, G at 919, C at 924, A at 930, G at 984, T at 993, G at 996, G at 1001, A at 1008, A at 1026, T at 1046, C at 1056, G at 1089, G at 1095, T at 1096, G at 1107, G at 1126, A at 1139, T at 1160, T at 1182, T at 1200, G at 1209, G at 1245, T at 1266, A at 1346, C at 1360, A at 1376, C at 1413, G at 1419, G at 1455, G at 1491, G at 1548, T at 1551, C at 1555, T at 1557, G at 1560, T at 1586, G at 1594, C at 1597, T at 1599, A at 1606, G at 1637, C at 1641, A at 1662, A at 1665, T at 1669, T at 1680, T at 1701, C at 1704, A at 1707, G at 1734, T at 1737, T at 1755, T at 1757, T at 1761, G at 1764, A at 1797, T at 1815, C at 1818, G at 1833, A at 1878, C at 1917, C at 1923, C at 1941, A at 1965, T at 2013, G at 2085, A at 2029, G at 2079, T at 2082, A at 2120, C at 2042, C at 2207, inserts: CAA between nucleotides 135 and 136 of the consensus sequence; CCA between nucleotides 223 and 224 of the consensus sequence; and deletions at positions: 138-140; 216-218.

FECV (SEQ ID NO: 32) differs from the Consensus Sequence (SEQ ID NO: 33) by the following amino acid changes: Ser at 18, Ser at 21, Asn at 24, Arg at 26, Gln at 46, Ser at 47, Ile at 53, Thr at 73, Tyr at 77, Glu at 78, Asp at 89, Ile at 116, Gly at 124, Glu at 134, Leu at 138, Asn at 145, Asp at 147, Asn at 149, Thr at 150, Asp at 157, Ile at 159, Asn at 163, Arg at 174, Glu at 188, Asn at 200, Trp at 201, Asn at 205, Val at 230, Phe at 253, Tyr at 259, Val at 272, Val at 307, Ser at 334, Val at 376, Asn at 380, Phe at 388, Asp at 449, Asp at 459, Lys at 485, Leu at 519, Ile at 529, Ala at 532, His at 533, Ile at 536, Arg at 546, Ile at 586, Glu at 598, Asp at 605, Asn at 677, Glu at 693, and Gln at 707.

In addition, WT WSU 1146 differs from the nucleotide sequence of FECV by the following changes (the WT WSU 1146 nucleotide and nucleotide numbers appear before the FECV nucleotides and nucleotide numbers which are in parentheses): T at 2271 (C at 2208); C at 2372 (A at 2309); T at 2376 (C at 2313); G at 2385 (A at 2322); C at 2421 (T at 2358); G at 2426 (A at 2363); G at 2479 (A at 2416); T at 2496 (C at 2433); C at 2550 (T at 2487); A at 2579 (C at 2516); T at 2598 (C at 2535); T at 2604 (C at 2541); T at 2619 (C at 2556); G at 2628 (T at 2565); T at 2640 (C at 2577); T at 2676 (C at 2613); G at 2718 (T at 2655); A at 2739 (G at 2676); T at 2796 (C at 2733); C at 2799 (T at 2736); G at 2802 (T at 2739); T at 2859 (C at 2796); G at 2882 (A at 2819); C at 2899 (T at 2836); C at 2908 (T at 2845); T at 2916 (C at 2853); A at 2922 (G at 2859); G at 2950 (C at 2887); T at 2967 (C at 2904); A at 2982 (G at 2919); A at 2991 (T at 2928); T at 3033 (A at 2970); C at 3042 (T at 2979); A at 3051 (C at 2988); G at 3057 (A at 2994); T at 3090 (G at 3027); C at 3091 (T at 3028); A at 3096 (T at 3033); C at 3110 (A at 3047); A at 3138 (T at 3075); T at 3157 (C at 3094); G at 3183 (T at 3120); A at 3207 (T at 3144); G at 3210 (A at 3147); A at 3261 (G at 3198); T at 3312 (A at 3249); T at 3318 (C at 3255); C at 3349 (A at 3286); C at 3360 (A at 3297); G at 3375 (A at 3312); T at 3423 (C at 3360); T at 3429 (A at 3366); T at 3468 (C at 3405); T at 3540 (A at 3477); A at 3591 (G at 3528); A at 3621 (G at 3558); G at 3645 (A at 3582); T at 3648 (C at 3585); G at 3651 (A at 3588); C at 3663 (T at 3600); T at 3687 (C at 3624); A at 3699 (T at 3636); A at 3741 (G at 3678); A at 3753 (G at 3690); T at 3778 (C at 3715); C at 3813 (T at 3750); G at 3834 (A at 3771); T at 3855 (C at 3792); C at 3879 (T at 3816); T at 3905 (C at 3842); A at 3936 (G at 3873); T at 3942 (C at 3879); C at 3960 (T at 3897); G at 3963 (A at 3900); T at 3975 (C at 3912); T at 4008 (C at 3945); A at 4014 (G at 3951); C at 4026 (T at 3963); T at 4068 (G at 4005); C at 4083 (T at 4020); G at 4128 (A at 4065); T at 4149 (C at 4086); C at 4152 (T at 4089); T at 4155 (C at 4092); A at 4158 (T at 4095); T at 4182 (C at 4119); T at 4191 (C at 4128); T at 4194 (C at 4131); G at 4266 (A at 4203); T at 4272 (C at 4209); G at 4282 (A at 4219); C at 4300 (T at 4237); T at 4316 (G at 4253); C at 4320 (T at 4257); T at 4347 (C at 4284); and A at 4371 (G at 4308). FECV differs from the amino acid sequence of WT WSU 1146 by the following changes (WSU 1146 amino acids appear in parentheses): Lys at 770 (Thr at 768); Asn at 788 (Ser at 786); Ile at 806 (Val at 804); Thr at 839 (Asn at 837); Ile at 855 (Met at 853); Asn at 940 (Ser at 938); Arg at 963 (Gly at 961); Asp at 1016 (Ala at 1014); Lys at 1096 (Gln at 1094); Pro at 1239 (Ser at 1237); Ala at 1281 (Val at 1279); Leu at 1335 (Phe at 1333); Ile at 1407 (Val at 1405); Cys at 1418 (Phe at 1416); and Met at 1436 (Ile at 1434).

UCD-2 (SEQ ID NO: 54) differs from the amino acid sequence of the Consensus Sequence by the following amino acid change: Tyr at #21, Ile at #22. The are no nucleotide differences between the UCD-2 nucleic acid sequence and the Consensus Sequence.

The following general conclusions can be drawn from this information. FECV and all of the viruses derived from WT DF2 contain a 2 amino acid insert (Tyr Ile) at positions #119 and 120 which is absent in the WT WSU 1146 S gene. In general, however, the homology between WT WSU 1146 and WT DF2 derived strains is quite high (>99.0%). Six changes exist in the first 748 amino acids of the DF2-HP S gene as compared to the WT DF2 sequence. The majority of the changes are conservative but several (r459, #533) may perturb protein conformation. The overall amino acid homology between DF2 HP and DF2 remains >99.0%.

In the first half of the S gene, mutagenesis of the DF2 HP could have caused the five amino acid changes observed in TS FIPV. Again, the majority of the changes are conservative in nature. However, the amino acid substitutions at position #553 and #630 may cause changes in the protein plot structure. Overall, the similarity of the two viruses is greater than 99.0%.

The 1-748 amino acid sequences of TS FIPV (SEQ ID NO: 26) and TS-BP (SEQ ID NO: 28) are highly homologous (>99.0%). However, comparison of TS FIPV (SEQ ID NO: 26) with TS BP (SEQ ID NO: 28) did show three amino acid changes. Two of these represented conservative changes, from valine to alanine at #319 and from isoleucine to valine at #662. Examination of the plot structures at these two amino acid positions predicts that these two changes will have minimal effect of the protein conformation. The third change at #630 is significant: from a tyrosine in TS FIPV to a lysine residue in the TS BP. While this amino acid change may perturb protein folding, the consensus amino acid at this portion in WT DF2 (SEQ ID NO: 22), DF2 HP (SEQ ID NO: 24) and FECV (SEQ ID NO: 32) is a lysine. This result suggests that the change back to a lysine in TS BP is not associated with a return to virulence.

Only one amino acid change (#220, tryptophan to isoleucine) was observed in the sequence of the WT TN406 94-223 amino acid region with respect to the other FIPV strains, which are all Type II. WT TN406 is a Type I virus and typically requires greater than one exposure to cause disease in cats. The illustrated TN406 sequence consists of nucleotides 302-671 [SEQ ID NO: 29] and amino acid numbers 102-223 [SEQ ID NO: 30].

### EXAMPLE 13

Challenge Studies

To further identify FIPV and FECV strains that contained S gene sequences sufficiently non-homologous to be capable of selectively distinguishing various FIPV strains from FECV, sera was screened from either rabbits immunized with synthetic peptides representing amino acids 137-151 or 150-180 or cats challenged with specific feline coronaviruses. The results are as follows. Sera from cats immunized with FIPV strains WT WSU 1146 or WT DF2 did not recognize a fusion protein representing amino acids 94-223 of FECV when probed on a Western blot. In contrast, a fusion protein representing amino acids 94-223 of TS FIPV was not recognized by sera from cats infected with FECV but was detected on a Western blot probes with sera from IT WSU 1146-infected or WT DF2-infected cat sera. Sera from rabbits immunized with a synthetic peptide made to the WT WSU 1146 amino acid sequence at positions 137-151 recognized only the TS FIPV but not the FECV 94-223 fusion protein. These results suggest that specific sequences, such as 137-151 amino acids, within the 94-223 fusion protein, may be useful in differentiating FIPV from FECV. As illustrated in the following Table V, both the TS FIPV and FECV 94-223 amino acid fusion proteins were recognized by galK monoclonal antibody HIV env 41 AS1 [Beckman Instruments].

TABLE V

| | | TS FIPV | TS FIPV |
|---|---|---|---|
| Challenge | Serum | AR 58-3 | AR 58-399 |
| Virus Type | Type | 93-223 aa | 94-223 aa |
| WSU 1146 | Post-Chall* | + | − |
| WT DF2 | Post-Chall | + | − |
| FECV | Post-Vac-3 | − | + |
| Rabbit | WT FIPV aa 137-151 | ++ | − |
| | WT FIPV aa 150-180 | ++ | NT |
| Mouse | Anti-GalK Mab | ++ | ++ |

+/− denotes reactivity on Western blot with cat sera
*symptomatic cats which died from FIPV after challenge
NT not tested

### EXAMPLE 14

Antibody Recognition of Non-Homologous Sequences

Synthetic peptides made from the WT DF2/WT WSU 1146 sequence at amino acid positions #137-151 and #950-990 (a control) were used to immunize rabbits. As illustrated in the following Table VI, the antibody directed against the 137-151 synthetic peptide recognized fusion proteins representing WT DF2 and TS FIPV 94-223 amino acids, but not the analogous fusion protein made from FECV. As predicted, the control antibody did not recognize any 94-223 a.a. fusion protein tested. The monoclonal gal-K antibody recognized the galactokinase portion of all fusion proteins. On the following illustration of the Western Blot results, a “0” indicates no reaction and a “4” indicates a strong reaction.

TABLE VI

| | TS FIPV | TN406 | FECV |
|---|---|---|---|
| Sera | 94-223 | 94-223 | 94-223 |
| Rabbit α 137-151 aa | 2 | 2 | 0 |
| Rabbit α 950-990 aa | 0 | 0 | 0 |
| Mouse anti-galK | 4 | 2 | 4 |

Numerous modifications and variations of the present invention are included in the above-identified specification and are expected to be obvious to one skilled in the art. Such modification and alterations are believed to be encompassed in the scope of the claims appended hereto.

What is claimed is:

1. A peptide or protein useful in the diagnosis, treatment or prophylaxis of a disease caused by a coronavirus or related virus comprising a selected sequence from the S gene of a coronavirus strain, optionally fused in frame to a gene sequence encoding a selected fusion partner protein or portion thereof.

2. The protein according to claim 1 wherein the S gene is obtained from the feline coronavirus, Feline Infectious Peritonitis Virus.

3. The protein according to claim 1 wherein said coronavirus is selected from the group consisting of WT FIPV DF2, WT FIPV WSU 1146, TS FIPV, WT FIPV UCD-2, WT FIPV TN406, WT FIPV UCD-1, FIPV DF2-HP, and FIPV TS-BP.

4. The protein according to claim 1 wherein the S gene is obtained from the feline coronavirus, FECV.

5. The protein according to claim 1 wherein said selected S gene sequence encodes a peptide comprising a sequence homologous to amino acid numbers 1-1454 of the said S protein, or a fragment thereof.

6. The protein according to claim 5 wherein said selected S gene sequence encodes a peptide comprising a sequence homologous to amino acid numbers 1-748 of said S protein, or a fragment thereof.

7. The protein according to claim 1 wherein said selected S gene sequence encodes a peptide comprising amino acid numbers 94-223 of said S protein.

8. The protein according to claim 1 comprising a peptide selected from the group consisting of amino acid numbers 18-26 [SEQ ID NO: 36], 46-53 [SEQ ID NO: 38], 73-78 [SEQ ID NO: 40], 124-174, 145-150 [SEQ ID NO: 42], 138-159 [SEQ ID NO: 44], 143-150 [SEQ ID NO: 46], 200-205 [SEQ ID NO: 48], and 529-536 [SEQ ID NO: 50] from FECV, corresponding peptides of FIPV, corresponding peptides of the consensus sequence, and fragments thereof, said peptides capable of distinguishing between FIPV strains and FECV.

9. The protein according to claim 1 wherein said selected fusion partner protein is selected from the group consisting of galactokinase, beta-galactosidase, ubiquitin, a mating factor, and influenza NS-1 or portions thereof.

10. The protein according to claim 9 wherein said selected fusion partner protein comprises the N-terminal 52 amino acids of galactokinase.

11. A peptide or protein useful in the diagnosis, treatment or prophylaxis of Feline Infectious Peritonitis Virus comprising a selected sequence from the S gene of a feline coronavirus strain, optionally fused in frame to a galactokinase gene or the N terminal 52 amino acids thereof.

12. A peptide or protein comprising all or a portion of the amino acid sequences selected from the group consisting of the amino acid sequences of FIG. 3 SEQ ID NO: 20, FIG. 4 SEQ ID NOS: 22 and 24, FIG. 5 SEQ ID NOS: 26 and 28, FIG. 6 SEQ ID NO: 30, FIG. 7 SEQ ID NO: 32, FIG. 8 SEQ ID NO: 54, and FIG. 9 SEQ ID NO: 34.

13. A DNA sequence useful in the diagnosis, treatment or prophylaxis of a disease caused by a coronavirus or related virus comprising a selected nucleotide sequence from the S gene of a feline coronavirus strain.

14. The DNA sequence according to claim 13 wherein the S gene is obtained from the feline coronavirus, Feline Infectious Peritonitis Virus.

15. The DNA sequence according to claim 13 wherein said coronavirus is selected from the group consisting of WT FIPV DF2, WT FIPV WSU 1146, TS FIPV, WT FIPV UCD-2, WT FIPV TN406, WT FIPV UCD-1, FIPV DF2-HP, and FIPV TS-BP.

16. The DNA sequence according to claim 13 wherein the S gene is obtained from the feline coronavirus, FECV.

17. The DNA sequence according to claim 13 comprising a sequence spanning nucleotides 1 to about 4365 of said S gene, or fragments thereof.

18. The DNA sequence according to claim 17 comprising a sequence spanning nucleotide numbers 1 to 2246 of said S gene, or a fragment thereof.

19. The DNA sequence according to claim 17 comprising a sequence spanning nucleotide numbers 1-370 of said S gene, or a fragment thereof.

20. The DNA sequence according to claim 13 selected from the group consisting of FECV nucleotide numbers 52-78 [SEQ ID NO: 35], 136-159 [SEQ ID NO: 37], 214-231 [SEQ ID NO: 39], 370-519 [SEQ ID NO: 41], 433-450 [SEQ ID NO: 43], 412-477 [SEQ ID NO: 45], 427-450 [SEQ ID NO: 47], 598-615 [SEQ ID NO: 49], and 1585-1608 [SEQ ID NO: 513 corresponding sequences of FIPV, corresponding sequences of the consensus sequence and fragments thereof, said sequences capable of distinguishing between FIPV strains and FECV.

21. A DNA sequence useful in the diagnosis, treatment or prophylaxis of Feline Infectious Peritonitis virus comprising a selected sequence from the S gene of a feline coronavirus strain, optionally fused in frame to a DNA sequence encoding the galactokinase gene or the N terminal 52 amino acids thereof.

22. A DNA sequence comprising all or a portion of the nucleotide sequences selected from the group consisting of the amino acid sequences of FIG. 3 SEQ ID NO: 19, FIG. 4 SEQ ID NO: 21 and 23, FIG. 5 SEQ ID NO: 25 and 27, FIG. 6 SEQ ID NO: 29, FIG. 7 SEQ ID NO: 31, FIG. 8 SEQ ID NO: 53, and FIG. 9 SEQ ID NO: 33.

23. A method for production of a recombinant protein useful in the diagnosis, treatment or prophylaxis of diseases caused by feline coronaviruses comprising culturing a selected host cell transformed with a DNA sequence encoding a selected sequence from the S gene of a feline coronavirus strain, optionally fused in frame to a gene sequence encoding a selected fusion partner in operative association with regulatory sequences capable of regulating the expression of said protein.

24. The method according to claim 23 wherein said fusion partner protein is selected from the group consisting of galactokinase, beta-galactosidase, ubiquitin, a mating factor, and influenza NS-1 or portions thereof.

25. The method according to claim 23 wherein the feline coronavirus is Feline Infectious Peritonitis Virus.

26. The method according to claim 23 wherein the feline coronavirus is FECV.

27. The method according to claim 23 wherein selected sequence comprises a peptide selected from the group consisting of amino acid numbers 18-26 [SEQ ID NO: 36], 46-53 [SEQ ID NO: 38], 73-78 [SEQ ID NO: 40], 124-174, 145-150 [SEQ ID NO: 42], 138-159 [SEQ ID NO: 44], 143-150 [SEQ ID NO: 46], 200-205 [SEQ ID NO: 48], and 529-536 [SEQ ID NO: 50] from FECV, corresponding peptides of FIPV, corresponding peptides of the consensus sequence, and fragments thereof, said peptides capable of distinguishing between FIPV strains and FECV.

28. The method according to claim 23 wherein said cells are bacterial cells.

29. The method according to claim 23 wherein said cells are E. coli cells.

30. The method according to claim 23 further comprising separating said coronavirus S fusion protein by affinity chromatography using monoclonal antibodies to said fusion partner protein or portion thereof.

31. A recombinant DNA molecule comprising a DNA sequence coding for a selected portion of a feline coronavirus S gene, optionally fused in frame to a DNA sequence encoding a selected portion of a fusion partner protein, said DNA sequences in operative association with regulatory sequences capable of directing the expression thereof in host cells.

32. The molecule according to claim 31 wherein said fusion partner protein is galactokinase, the N-terminal 52 amino acids thereof, beta-galactosidase, ubiquitin, a mating factor, and influenza NS-1 or portions thereof.

33. The molecule according to claim 31 wherein said DNA sequence is selected from the group consisting of FECV nucleotide numbers 52-78 [SEQ ID NO: 35], 136-159 [SEQ ID NO: 37], 214-231 [SEQ ID NO: 39], 370-519 [SEQ ID NO: 41], 433-450 [SEQ ID NO: 43], 412-477 [SEQ ID NO: 45], 427-450 [SEQ ID NO: 47], 598-615 [SEQ ID NO: 49], and 1585-1608 [SEQ ID NO: 51], corresponding sequences of FIPV, corresponding sequences of a consensus sequence, and fragments thereof, said sequences capable of distinguishing between FIPV strains and FECV.

34. The molecule according to claim 31 comprising pOTSKF33.

35. A galactokinase-feline coronavirus S fusion protein gene expression unit comprising a DNA sequence encoding said protein, and a regulatory sequence capable of directing the transcription of the protein coding sequence and subsequent translation within a bacterial cell.

36. A vaccine composition comprising an immunogenic amount of a feline coronavirus protein comprising a selected sequence from the S gene of a feline coronavirus strain, optionally fused in frame to a gene sequence encoding a selected fusion partner protein or portion thereof and an optional carrier.

37. The vaccine composition according to claim 36 wherein said fusion partner comprises the first 52 N-terminal amino acids of galactokinase.

38. The vaccine composition according to claim 36 comprising at least 1-10 feline coronavirus S fusion proteins per ml.

39. The vaccine composition according to claim 36 further comprising an immunogenic amount of one or more additional antigens.

40. The vaccine composition according to claim 39 wherein said additional antigens are feline antigens.

41. The vaccine composition according to claim 39 wherein said feline antigens are coronaviruses.

42. The vaccine composition according to claim 37 further comprising a temperature sensitive FIPV antigen.

43. The vaccine composition according to claim 37 comprising a dosage unit of 0.1 μg to 100 μg per ml of a sterile solution of an immunogenic amount of a galactokinase-feline coronavirus S protein, optionally fused in frame to a gene sequence encoding a selected fusion partner protein or portion thereof.

44. A method for vaccinating a naive animal against Feline Infectious Peritonitis Virus which comprises internally administering to the animal an effective immunogenic amount of a protein according to claim 1.

45. A method for vaccinating a naive animal against Feline Infectious Peritonitis Virus which comprises internally administering to the animal a vaccine composition of claim 42.

46. A pharmaceutical composition for treating Feline Infectious Peritonitis Virus infection in an infected animal comprising an effective non-toxic amount of a feline coronavirus protein comprising a selected sequence from the S gene of a feline coronavirus strain, optionally fused in frame to a gene sequence encoding a selected fusion partner protein or portion thereof and an optional pharmaceutical carrier.

47. A diagnostic kit for distinguishing between native FIPV exposure and vaccinated animals and between first and second FIPV exposure in FIPV-infected animals comprising a protein according to claim 1 or a DNA sequence according to claim 13.

48. A diagnostic agent comprising a protein of claim 1, a primer sequence of Table II (SEQ ID NO: 1 through SEQ ID NO: 18), or a DNA sequence according to claim 13, optionally associated with a detectable label, said agent capable of detecting a selected coronavirus in a biological sample.

49. A method for distinguishing one coronavirus from another coronavirus, including one species coronavirus from another species coronavirus, comprising employing a protein of claim 1, a primer sequence of Table II (SEQ ID NO: 1 through SEQ ID NO: 18), or a DNA sequence according to claim 13.

50. An antibody to a peptide or protein according to claim 1, said antibody capable directed to an epitope capable of distinguish FIPV strains and FECV.


SEQUENCE LISTING (54 sequences)

SEQ ID NO: 1 - DNA (genomic), 39 bp; double-stranded, unknown
gtgcccccgggtatgattgtgctcgtaacttgcctcttg

SEQ ID NO: 2 - DNA (genomic), 35 bp; single-stranded, unknown
aaatacccgggcactggtaatgcacgtggtaaacc

SEQ ID NO: 3 - DNA (genomic), 35 bp; single-stranded, unknown
gtattcccgggcacgctcaagcactgctacctggg

SEQ ID NO: 4 - DNA (genomic), 36 bp; single-stranded, unknown
cagatcccggggtacaatctggtatgggtgctacag

SEQ ID NO: 5 - DNA (genomic), 39 bp; single-stranded, unknown
gcttacccggggtggttatggtcaacccatagcctcgac

SEQ ID NO: 6 - DNA (genomic), 39 bp; single-stranded, unknown
tgtgacccgggcgccatgtgatgtaagcgcacaagcggc

SEQ ID NO: 7 - DNA (genomic), 37 bp; single-stranded, unknown
gcaatcccggggggtgccagacttgaaaacatggagg

SEQ ID NO: 8 - DNA (genomic), 37 bp; single-stranded, unknown
cattacccgggggtgcacttggtggtggcgccgtggc

SEQ ID NO: 9 - DNA (genomic), 39 bp; single-stranded, unknown
taggtcccgggctcagtctcagagattcggattctgtgg

SEQ ID NO: 10 - DNA (genomic), 36 bp; single-stranded, unknown
ataataggcctggtttaccacgtgcattaccagtgc

SEQ ID NO: 11 - DNA (genomic), 35 bp; single-stranded, unknown
gtattaggcctcccaggtagcagtgcttgagcgtg

SEQ ID NO: 12 - DNA (genomic), 36 bp; single-stranded, unknown
aaataaggcctctgtagcacccataccagattgtac

SEQ ID NO: 13 - DNA (genomic), 39 bp; single-stranded, unknown
ttagtaggcctgtcgaggctatgggttgaccataaccac

SEQ ID NO: 14 - DNA (genomic), 39 bp; single-stranded, unknown
taacaaggcctgccgcttgtgcgcttacatcacatggcg

SEQ ID NO: 15 - DNA (genomic), 37 bp; single-stranded, unknown
atcaaaggcctcctccatgttttcaagtctggcaccc

SEQ ID NO: 16 - DNA (genomic), 37 bp; single-stranded, unknown
gtataaggcctgccacggcgccaccaccaagtgcacc

SEQ ID NO: 17 - DNA (genomic), 39 bp; single-stranded, unknown
cattaaggcctccacagaatccgaatctctgagactgag

SEQ ID NO: 18 - DNA (genomic), 38 bp; single-stranded, unknown
taaataggcctttagtggacatgcactttttcaattgg

SEQ ID NO: 19 - DNA (cDNA), 573 bp; double-stranded, unknown
  CDS 1..570
atggatcccgaattccaagaaaaaacacaatctctgtttgccaacgcatttggctaccctgccactcacaccattcagggccctggccgcgtgaatttgattggtgaacacaccgactacaacgacggtttcgttctgccctgcgcgattgattatcaaaccgtgatccctaatacccggggcactggtaatgcacgtggtaaaccattattatttcatgtgcatggtgagcctgttagtgttattatatatatatcggcttatagggatgatgtgcaacaaaggccccttttaaaacatgggttagtgtgtataactaaaaatcgccatattaactatgaacaattcgcctccaaccagtggaattccacatgtacgggtgctgacagaaaaattcccttctctgtcatacccacggacaatggaacaaaaatctatggtcttgagtggaatgatgactttgttacagcttatattagtggtcgttcttatcacttgaacatcaatactaattggtttaacaatgtcacacttttgtattcacgcagcagcactgctacctgggaggcctag

SEQ ID NO: 20 - protein, 190 aa
MDPEFQEKTQSLFANAFGYPATHTIQGPGRVNLIGEHTDYNDGFVLPCAIDYQTVIPNTRGTGNARGKPLLFHVHGEPVSVIIYISAYRDDVQQRPLLKHGLVCITKNRHINYEQFASNQWNSTCTGADRKIPFSVIPTDNGTKIYGLEWNDDFVTAYISGRSYHLNINTNWFNNVTLLYSRSSTATWEA

SEQ ID NO: 21 - DNA (cDNA), 4365 bp; double-stranded, unknown
  CDS 1..4362
atgattgtgctcgtaacttgcctcttgttgttatgttcataccacacagttttgagtacaacaaataatgaatgcatacaagttaacgtaacacaattggctggcaatgaaaaccttatcagagattttctgtttagtaactttaaagaagaaggaagtgtagttgttggtggttattaccctacagaggtgtggtacaactgctctagaacagcacaaactactgcctttcagtattttaataatatacatgccttttattttgttatggaagccatggaaaatagcactggtaatgcacgtggtaaaccattattatttcatgtgcatggtgagcctgttagtgttattatatatatatcggcttatagggatgatgtgcaacaaaggccccttttaaaacatgggttagtgtgcataactaaaaatcgccatattaactatgaacaattcacctccaaccagtggaattccacatgtacgggtgctgacagaaaaattcctttctctgtcatacccacggacaatggaacaaaaatctatggtcttgagtggaatgatgactttgttacagcttatattagtggtcgttcttatcacttgaacatcaatactaattggtttaacaatgtcacacttttgtattcacgctcaagcactgctacctgggaatacagtgctgcatatgcttaccaaggtgtttctaacttcacttattacaagttaaataacaccaatggtctaaaaacctatgaattatgtgaagattatgaacattgcactggctatgctaccaatgtatttgctccgacatcaggtggttacatacctgatggatttagttttaacaattggttcttgcttacaaatagttccacttttgttagtggcaggtttgtaacaaatcaaccattattgattaattgcttgtggccagtgcccagttttggtgtagcagcacaagaattttgttttgaaggtgcacagtttagccaatgtaatggtgtgtctttaaataacacagtggatgttattagattcaaccttaatttcactgcagatgtacaatctggtatgggtgctacagtattttcactgaatacaacaggtggtgtcattcttgaaatttcatgttatagtgacacagtgagtgagtctagttcttacagttatggtgaaatcccgttcggcataactgacggaccacgatactgttatgtactttacaatggcacagctcttaaatatttaggaacattaccacccagtgtaaaggaaattgctattagtaagtggggccatttttatattaatggttacaatttctttagcacatttcctattggttgtatatcttttaatttaaccactggtgctagtggagctttttggacaattgcttacacatcgtatactgaagcattagtacaagttgaaaacacagctattaaaaatgtgacgtattgtaacagtcacattaataacattaaatgttctcaacttactgctaatttgaataatggattttatcctgttgcttcaagtgaagtaggtttcgttaataagagtgttgtgttattacctagctttttcacacacaccgctgtcaatataaccattgatcttggtatgaagcttagtggttatggtcaacccatagcctcgacactaagtaacatcacactaccaatgcaggataacaatactgatgtgtactgtattcgttctaaccaattctcagtttatgttccttccacttgcaaaagttctttatgggacaatatttttaatcaagactgcacggatgttttagaggctacagctgttataaaaactggtacttgtcctttctcatttgataaattgaacaattacttgacttttaacaagttctgtttgtcgttgagtcctgttggtgctaattgcaagtttgatgttgctgcacgtacaagaaccaatgagcaggttgttagaagtctatatgtaatatatgaagaaggagacaacatagtgggtgtaccgtctgataatagcggtctgcacgatttgtctgtgctacacctagactcctgtacagattacaatatatatggtagaactggtgttggtattattagacgaactaacagtacgctacttagtggcttatattacacatcactatcaggtgatttgttaggctttaaaaatgttagtgatggtgtcatttattctgtgacgccatgtgatgtaagcgcacaagcggctgttattgatggtgccatagttggagctatgacttccattaacagtgaactgttaggtctaacacattggacaacgacacctaatttttattactactctatatataattacacaagtgagaggactcgtggcactgcaattgacagtaacgatgttgattgtgaacctgtcataacctattctaatataggtgtttgtaaaaatggtgctttggtttttattaacgtcacacattctgacggagacgtgcaaccaattagcactggtaatgtcacgatacctacaaattttaccatatctgtgcaagttgaatacatgcaggtttacactacaccagtatcaatagattgtgcaagatacgtttgtaatggtaaccctagatgtaacaaattgttaacacaatatgtgtctgcatgtcaaactattgaacaagcacttgcaatgggtgccagacttgaaaacatggaggttgattccatgttgtttgtctcggaaaatgcccttaaattggcatctgttgaggcgttcaatagtacagaaaatttagatcctatttacaaagaatggcctagcataggtggttcttggctaggaggtctaaaagatatactaccgtcccataatagcaaacgtaagtatggttctgctatagaagatttgctttttgataaagttgtaacatctggtttaggtacagttgatgaagattataaacgttgtactggtggttacgacatagcagacttggtgtgtgctcaatattacaatggcatcatggttctaccaggtgtagctaatgctgacaagatgactatgtacacagcatcacttgcaggtggtataacattaggtgcacttggtggtggcgccgtggctataccttttgcagtagcagtacaggctagacttaattatgttgctctacaaactgatgtattgaataaaaaccaacagatcctggctaatgctttcaatcaagctattggtaacattacacaggcttttggtaaggttaatgatgctatacatcaaacatcacaaggtcttgccactgttgctaaagcgttggcaaaagtgcaagatgttgtcaacacacaagggcaagctttaagtcaccttacagtacaattgcaaaataattttcaagccattagtagttctattagtgatatttataacaggcttgacgaactgagtgctgatgcacaagttgataggctgattacaggtagacttacagcacttaatgcatttgtgtctcagactctaaccagacaagcagaggttagggctagtagacaacttgccaaagacaaggttaatgaatgtgttaggtctcagtctcagagattcggattctgtggtaatggtacacatttgttttcactagcaaatgcagcaccaaatggcatgattttctttcatacagtactattaccaacagcttatgaaactgtaacagcttggtcaggtatttgtgcttcagatggcgatcgcactttcggacttgtcgttaaagatgtgcagttgacgttgtttcgtaatctagatgacaagttctatttgacccccagaactatgtatcagcctagagttgcaactagttctgattttgttcaaattgaagggtgtgatgtgttgtttgtcaacgcgactgtaattgatttgcctagtattatacctgactatattgacattaatcaaactgttcaagacatattagaaaattacagaccaaactggactgtacctgaatttacacttgatattttcaacgcaacctatttaaatctgactggtgaaattgatgacttagagtttaggtcagaaaagctacataacactacagtagaacttgccattctcattgataccattaataatacattagtcaatcttgaatggctcaatagaattgaaacttatgtaaaatggccttggtatgtgtggctactgataggtctagtagtagtattttgcataccattactgctattttgctgttttagcacaggttgttgtggatgcataggttgtttaggaagttgttgtcactctatatgtagtagaagacaatttgaatattatgaaccaattgaaaaagtgcatgtccactaa

SEQ ID NO: 22 - protein, 1454 aa
MIVLVTCLLLLCSYHTVLSTTNNECIQVNVTQLAGNENLIRDFLFSNFKEEGSVVVGGYYPTEVWYNCSRTAQTTAFQYFNNIHAFYFVMEAMENSTGNARGKPLLFHVHGEPVSVIIYISAYRDDVQQRPLLKHGLVCITKNRHINYEQFTSNQWNSTCTGADRKIPFSVIPTDNGTKIYGLEWNDDFVTAYISGRSYHLNINTNWFNNVTLLYSRSSTATWEYSAAYAYQGVSNFTYYKLNNTNGLKTYELCEDYEHCTGYATNVFAPTSGGYIPDGFSFNNWFLLTNSSTFVSGRFVTNQPLLINCLWPVPSFGVAAQEFCFEGAQFSQCNGVSLNNTVDVIRFNLNFTADVQSGMGATVFSLNTTGGVILEISCYSDTVSESSSYSYGEIPFGITDGPRYCYVLYNGTALKYLGTLPPSVKEIAISKWGHFYINGYNFFSTFPIGCISFNLTTGASGAFWTIAYTSYTEALVQVENTAIKNVTYCNSHINNIKCSQLTANLNNGFYPVASSEVGFVNKSVVLLPSFFTHTAVNITIDLGMKLSGYGQPIASTLSNITLPMQDNNTDVYCIRSNQFSVYVPSTCKSSLWDNIFNQDCTDVLEATAVIKTGTCPFSFDKLNNYLTFNKFCLSLSPVGANCKFDVAARTRTNEQVVRSLYVIYEEGDNIVGVPSDNSGLHDLSVLHLDSCTDYNIYGRTGVGIIRRTNSTLLSGLYYTSLSGDLLGFKNVSDGVIYSVTPCDVSAQAAVIDGAIVGAMTSINSELLGLTHWTTTPNFYYYSIYNYTSERTRGTAIDSNDVDCEPVITYSNIGVCKNGALVFINVTHSDGDVQPISTGNVTIPTNFTISVQVEYMQVYTTPVSIDCARYVCNGNPRCNKLLTQYVSACQTIEQALAMGARLENMEVDSMLFVSENALKLASVEAFNSTENLDPIYKEWPSIGGSWLGGLKDILPSHNSKRKYGSAIEDLLFDKVVTSGLGTVDEDYKRCTGGYDIADLVCAQYYNGIMVLPGVANADKMTMYTASLAGGITLGALGGGAVAIPFAVAVQARLNYVALQTDVLNKNQQILANAFNQAIGNITQAFGKVNDAIHQTSQGLATVAKALAKVQDVVNTQGQALSHLTVQLQNNFQAISSSISDIYNRLDELSADAQVDRLITGRLTALNAFVSQTLTRQAEVRASRQLAKDKVNECVRSQSQRFGFCGNGTHLFSLANAAPNGMIFFHTVLLPTAYETVTAWSGICASDGDRTFGLVVKDVQLTLFRNLDDKFYLTPRTMYQPRVATSSDFVQIEGCDVLFVNATVIDLPSIIPDYIDINQTVQDILENYRPNWTVPEFTLDIFNATYLNLTGEIDDLEFRSEKLHNTTVELAILIDTINNTLVNLEWLNRIETYVKWPWYVWLLIGLVVVFCIPLLLFCCFSTGCCGCIGCLGSCCHSICSRRQFEYYEPIEKVHVH

SEQ ID NO: 23 - DNA (cDNA), 2246 bp; double-stranded, unknown
  CDS 1..2244
atgattgtgctcgtaacttgcctcttgttgttatgttcataccacacagttttgagtacaacaaataatgaatgcatacaagttaacgtaacacaattggctggcaatgaaaaccttatcagagattttctgtttagtaactttaaagaagaaggaagtgtagttgttggtggttattaccctacagaggtgtggtacaactgctctagaacagctcgaactactgcctttcagtattttaataatatacatgccttttattttgttatggaagccatggaaaatagcactggtaatgcacgtggtaaaccattattatttcatgtgcatggtgagcctgttagtgttattatatatatatcggcttatagggatgatgtgcaacaaaggccccttttagaacatgggttagtgtgcataactaaaaatcgccatattaactatgaacaattcacctccaaccagtggaattccacatgtacgggtgctgacagaaaaattcctttctctgtcatacccacggacaatggaacaaaaatctatggtcttgagtggaatgatgactttgttacagcttatattagtggtcgttcttatcacttgaacatcaatactaattggtttaacaatgtcacacttttgtattcacgctcaagcactgctacctgggaatacagtgctgcatatgcttaccaaggtgtttctaacttcacttattacaagttaaataacaccaatggtctaaaaacctatgaattatgtgaagattatgaacattgcactggctatgctaccaatgtatttgctccgacatcaggtggttacatacctgatggatttagttttaataattggttcttgcttacaaatagttccacttttgttagtggcaggtttgtaacaaatcaaccattattgattaattgcttgtggccagtgcccagttttggtgtagcagcacaagaattttgttttgaaggtgcacagtttagccaatgtaatggtgtgtctttaaataacacagtggatgttattagattcaaccttaatttcactgcagatgtacaatctggtatgggtgccacagtattttcactgaatacaacaggtggtgtcattcttgaaatttcatgttatagtgacacagtgagtgagtctagttcttacagttatggtgaaatcccgttcggcataactgacggaccacgatactgttatgtactttacaatggcacagctcttaaatatttaggaacattaccacccagtgtaaaggaaattgctattagtaagtggggccatttttatattaatggttacaatttctttagcacatttcctattggttgtatatcttttaatttaaccactggtgttagtggagctttttggacaattgcttacacatcgtatactgaagcattagtacaagttgaaaacacagctattaaaaatgtgacgtattgtaacagtcacattaataacattaaatgttctcaacttactgctaatttgaataatggattttatcctgttgcttcaagtgaagtaggtttcgttaataagagtgttgtgttattacctagctttttcacatacaccgctgtcaatataaccattgatcttggtatgaagcttagtggttatggtcaacccatagcctcgacactaagtaacatcacactaccaatgcaggataacaatactgatgtgtactgtattcgttctaaccaattctcagtttatgttcattccacttgcaaaagttctttatgggacaatatctttaatcaagactgcacggatgttttagaggctacagctgttataaaaactggtacttgtcctttctcatttgataaattgaacaattacttgacttttaacaagttctgtttgtcgttgagtcctgttggtgctaattgcaagtttgatgttgctgcacgtacaagaaccaatgagcaggttgttagaagtctatatgtaatatatgaagaaggagacaacatagtgggtgtaccgtctgatgatagcggtctgcacgatttgtctgtgctacacctagactcctgtacagattacaatatatatggtagaactggtgttggtattattagacgaactaacagtacgctacttagtggcttatattacacatcactatcaggtgatttgttaggctttaaaaatgttagtgatggtgtcatttattctgtgacgccatgtgatgtaagcgcacaagcggc

SEQ ID NO: 24 - protein, 748 aa
MIVLVTCLLLLCSYHTVLSTTNNECIQVNVTQLAGNENLIRDFLFSNFKEEGSVVVGGYYPTEVWYNCSRTARTTAFQYFNNIHAFYFVMEAMENSTGNARGKPLLFHVHGEPVSVIIYISAYRDDVQQRPLLEHGLVCITKNRHINYEQFTSNQWNSTCTGADRKIPFSVIPTDNGTKIYGLEWNDDFVTAYISGRSYHLNINTNWFNNVTLLYSRSSTATWEYSAAYAYQGVSNFTYYKLNNTNGLKTYELCEDYEHCTGYATNVFAPTSGGYIPDGFSFNNWFLLTNSSTFVSGRFVTNQPLLINCLWPVPSFGVAAQEFCFEGAQFSQCNGVSLNNTVDVIRFNLNFTADVQSGMGATVFSLNTTGGVILEISCYSDTVSESSSYSYGEIPFGITDGPRYCYVLYNGTALKYLGTLPPSVKEIAISKWGHFYINGYNFFSTFPIGCISFNLTTGVSGAFWTIAYTSYTEALVQVENTAIKNVTYCNSHINNIKCSQLTANLNNGFYPVASSEVGFVNKSVVLLPSFFTYTAVNITIDLGMKLSGYGQPIASTLSNITLPMQDNNTDVYCIRSNQFSVYVHSTCKSSLWDNIFNQDCTDVLEATAVIKTGTCPFSFDKLNNYLTFNKFCLSLSPVGANCKFDVAARTRTNEQVVRSLYVIYEEGDNIVGVPSDDSGLHDLSVLHLDSCTDYNIYGRTGVGIIRRTNSTLLSGLYYTSLSGDLLGFKNVSDGVIYSVTPCDVSAQA

SEQ ID NO: 25 - DNA (cDNA), 4365 bp; double-stranded, unknown
  CDS 1..4362
atgattgtgctcgtaacttgcctcttgttgttatgttcataccacacagttttgagtacaacaaataatgaatgcatacaagttaacgttacacaattggctggcaatgaaaaccttatcagagattttctgtttagtaactttaaagaagaaggaagtgtagttgttggtggttattaccctacagaggtgtggtacaactgctctagaacagctcgaactactgcctttcagtattttaataatatacatgccttttattttgttatggaagccatggaaaatagcactggtaatgcacgtggtaaaccattattatttcatgtgcatggtgagcctgttagtgttattatatatatatcggcttatagggatgatgtgcaacaaaggccccttttaaaacatgggttagtgtgcataactaaaaatcgccatattaactatgaacaattcacctccaaccagtggaattccacatgtacgggtgctgacagaaaaattcctttctctgtcatacccacggacaatggaacaaaaatctatggtcttgagtggaatgatgactttgttacagcttatattagtggtcgttcttatcacttgaacatcaatactaattggtttaacaatgtcacacttttgtattcacgctcaagcactgctacctgggaatacagtgctgcatatgcttaccaaggtgtttctaacttcacttattacaagttaaataacaccaatggtctaaaaacctatgaattatgtgaagattatgaacattgcactggctatgctaccaatgtatttgctccgacatcaggtggttacatacctgatggatttagttttaataattggttcttgcttacaaatagttccacttttgttagtggcaggtttgtaacaaatcaaccattattgattaattgcttgtggccagtgcccagttttggtgtagtagcacaagaattttgttttgaaggtgcacagtttagccaatgtaatggtgtgtctttaaataacacagtggatgttattagattcaaccttaatttcactgcagatgtacaatctggtatgggtgctacagtattttcactgaatacaacaggtggtgtcattcttgaaatttcatgttatagtgacacagtgagtgagtctagttcttacagttatggtgaaatcccgttcggcataactgacggaccacgatactgttatgtactttacaatggcacagctcttaaatatttaggaacattaccacccagtgtaaaggaaattgctattagtaagtggggccatttttatattaatggttacaatttctttagcacatttcctattgattgtatatcttttaatttaaccactggtgttagtggagctttttggacaattgcttacacatcgtatactgaagcattagtacaagttgaaaacacagctattaaaaatgtgacgtattgtaacagtcacattaataacattaaatgttctcaacttactgctaatttgaataatggattttatcctgttgcttcaagtgaagtaggtttcgttaataagagtgttgtgttattacctagctttttcacatacaccgctgtcaatataaccattgatcttggtatgaagcttagtggttatggtcaacccatagcctcgacactaagtaacatcacactaccaatgcaggataacaatactgatgtgtactgtattcgttctaaccaattctcagtttatgttcattccacttgcaaaagttctttatgggacaatatttttaatcaagactgcacggatgttttagaggctacagctgttataaaaactggtacttgtcctttctcatttgataaattgaacaattacttgacttttaacacgttctgtttgtcgttgagtcctgttggtgctaattgcaagtttgatgttgctgcacgtacaagaaccaatgagcaggttgttagaagtctatatataatatatgaagaaggagacaacatagtgggtgtaccgtctgatgatagcggtctgcacgatttgtctgtgctacacctagactcctgtacagattacaatatatatggtagaactggtgttggtattattagacgaactaacagtacgctacttagtggcttatattacacatcactatcaggtgatttgttaggctttaaaaatgttagtgatggtgtcatttattctgtgacgccatgtgatgtaagcgcacaagcggctgttattgatggtgccatagttggagctatgacttccattaacagtgaactgttaggtctaatacattggacaacgacacctaatttttattactactctatatataattacacaagtgagaggactcgtggcactgcaattgacagtaacgatgttgattgtgaacctgtcataacctattctaatataggtgtttgtaaaaatggtgctttggtttttattaacgtcacacattctgacggagacgtgcaaccaattagcactggtaatgtcacgatacctacaaattttaccatatctgtgcaagttgaatacatgcaggtttacactacaccagtatcaatagattgtgcaagatacgtttgtaatggtaaccctagatgtaacaaattgttaacacaatatgtgtctgcatgtcaaactattgaacaagcacttgcaatgggtgccagacttgaaaacatggaggttgattccatgttgtttgtctcggaaaatgcccttaaattggcatctgttgaggcgttcaatagtacagaaaatttagatcctatttacaaagaatggcctagcataggtggttcttggctaggaggtctaaaagatatactaccgtcccataatagcaaacgtaagtatggttctgctatagaagatttgctttttgataaagttgtaacatctggtttaggtacagttgatgaagattataaacgttgtactggtggttacgacatagcagacttggtgtgtgctcaatattacaatggcatcatggttctaccaggtgtagctaatgctgacaagatgactatgtacacagcatcacttgcaggtggtataacattaggtgcacttggtggtggcgccgtggctataccttttgcagtagcagtacaggctagacttaattatgttgctctacaaactgatgtattgaataaaaaccaacagatcctggctaatgctttcaatcaagctattggtaacattacacaggcttttggtaaggttaatgatgctatacatcaaacatcacaaggtcttgccactgttgctaaagcgttggcaaaagtgcaagatgttgtcaacacacaagggcaagctttaagtcaccttacagtacaattgcaaaataattttcaagccattagtagttctattagtgatatttataacaggcttgacgaactgagtgctgatgcacaagttgataggctgattacaggtagacttacagcacttaatgcatttgtgtctcagactctaaccagacaagcagaggttagggctagtagacaacttgccaaagacaaggttaatgaatgtgttaggtctcagtctcagagattcggattctgtggtaatggtacacatttgttttcactagcaaatgcagcaccaaatggcatgattttctttcatacagtactattaccaacagcttatgaaactgtaacagcttggtcaggtatttgtgcttcagatggcgatcgcactttcggacttgtcgttaaagatgtgcagttgacgttgtttcgtaatctagatgacaagttctatttgacccccagaactatgtatcagcctagagttgcaactagttctgattttgttcaaattgaagggtgtgatgtgttgtttgtcaacgcgactgtaattgatttgcctagtattatacctgactatattgacattaatcaaactgttcaagacatattagaaaattacagaccaaactggactgtacctgaatttacacttgatattttcaacacaacctatttaaatctgactggtgaaattgatgacttagagtttaggtcggaaaagctacataacactacagtagaacttgccattctcattgataacattaataatacattagtcaatcttgaatggctcaatagaattgaaacttatgtaaaatggccttggtatgtgtggctactgataggtttagtagtagtattttgcataccattactgctattttgctgttttagcacaggttgttgtggatgcataggttgtttaggaagttgttgtcactctatatgtagtagaagacaatttgaaaattatgaaccaattgaaaaagtgcatgtccactaa

SEQ ID NO: 26 - protein, 1454 aa
MIVLVTCLLLLCSYHTVLSTTNNECIQVNVTQLAGNENLIRDFLFSNFKEEGSVVVGGYYPTEVWYNCSRTARTTAFQYFNNIHAFYFVMEAMENSTGNARGKPLLFHVHGEPVSVIIYISAYRDDVQQRPLLKHGLVCITKNRHINYEQFTSNQWNSTCTGADRKIPFSVIPTDNGTKIYGLEWNDDFVTAYISGRSYHLNINTNWFNNVTLLYSRSSTATWEYSAAYAYQGVSNFTYYKLNNTNGLKTYELCEDYEHCTGYATNVFAPTSGGYIPDGFSFNNWFLLTNSSTFVSGRFVTNQPLLINCLWPVPSFGVVAQEFCFEGAQFSQCNGVSLNNTVDVIRFNLNFTADVQSGMGATVFSLNTTGGVILEISCYSDTVSESSSYSYGEIPFGITDGPRYCYVLYNGTALKYLGTLPPSVKEIAISKWGHFYINGYNFFSTFPIDCISFNLTTGVSGAFWTIAYTSYTEALVQVENTAIKNVTYCNSHINNIKCSQLTANLNNGFYPVASSEVGFVNKSVVLLPSFFTYTAVNITIDLGMKLSGYGQPIASTLSNITLPMQDNNTDVYCIRSNQFSVYVHSTCKSSLWDNIFNQDCTDVLEATAVIKTGTCPFSFDKLNNYLTFNTFCLSLSPVGANCKFDVAARTRTNEQVVRSLYIIYEEGDNIVGVPSDDSGLHDLSVLHLDSCTDYNIYGRTGVGIIRRTNSTLLSGLYYTSLSGDLLGFKNVSDGVIYSVTPCDVSAQAAVIDGAIVGAMTSINSELLGLIHWTTTPNFYYYSIYNYTSERTRGTAIDSNDVDCEPVITYSNIGVCKNGALVFINVTHSDGDVQPISTGNVTIPTNFTISVQVEYMQVYTTPVSIDCARYVCNGNPRCNKLLTQYVSACQTIEQALAMGARLENMEVDSMLFVSENALKLASVEAFNSTENLDPIYKEWPSIGGSWLGGLKDILPSHNSKRKYGSAIEDLLFDKVVTSGLGTVDEDYKRCTGGYDIADLVCAQYYNGIMVLPGVANADKMTMYTASLAGGITLGALGGGAVAIPFAVAVQARLNYVALQTDVLNKNQQILANAFNQAIGNITQAFGKVNDAIHQTSQGLATVAKALAKVQDVVNTQGQALSHLTVQLQNNFQAISSSISDIYNRLDELSADAQVDRLITGRLTALNAFVSQTLTRQAEVRASRQLAKDKVNECVRSQSQRFGFCGNGTHLFSLANAAPNGMIFFHTVLLPTAYETVTAWSGICASDGDRTFGLVVKDVQLTLFRNLDDKFYLTPRTMYQPRVATSSDFVQIEGCDVLFVNATVIDLPSIIPDYIDINQTVQDILENYRPNWTVPEFTLDIFNTTYLNLTGEIDDLEFRSEKLHNTTVELAILIDNINNTLVNLEWLNRIETYVKWPWYVWLLIGLVVVFCIPLLLFCCFSTGCCGCIGCLGSCCHSICSRRQFENYEPIEKVHVH

SEQ ID NO: 27 - DNA (cDNA), 2246 bp; double-stranded, unknown
  CDS 1..2244
atgattgtgctcgtaacttgcctcttgttgttatgttcataccacacagttttgagtacaacaaataatgaatgcatacaagttaacgtaacacaattggctggcaatgaaaaccttatcagagattttctgtttagtaactttaaagaagaaggaagtgtagttgttggtggttattaccctacagaggtgtggtacaactgctctagaacagctcgaactactgcctttcagtattttaataatatacatgccttttattttgttatggaagccatggaaaatagcactggtaatgcacgtggtaaaccattattatttcatgtgcatggtgagcctgttagtgttattatatatatatcggcttatagggatgatgtgcaacaaaggccccttttaaaacatgggttagtgtgcataactaaaaatcgccatattaactatgaacaattcacctccaaccagtggaattccacatgtacgggtgctgacagaaaaattcctttctctgtcatacccacggacaatggaacaaaaatctatggtcttgagtggaatgatgactttgttacagcttatattagtggtcgttcttatcacttgaacatcaatactaattggtttaacaatgtcacacttttgtattcacgctcaagcactgctacctgggaatacagtgctgcatatgcttaccaaggtgtttctaacttcacttattacaagttaaataacaccaatggtctaaaaacctatgaattatgtgaagattatgaacattgcactggctatgctaccaatgtatttgctccgacatcaggtggttacatacctgatggatttagttttaataattggttcttgcttacaaatagttccacttttgttagtggcaggtttgtaacaaatcaaccattattgattaattgcttgtggccagtgcccagttttggtgtagcagcacaagaattttgttttgaaggtgcacagtttagccaatgtaatggtgtgtctttaaataacacagtggatgttattagattcaaccttaatttcactgcagatgtacaatctggtatgggtgctacagtattttcactgaatacaacaggtggtgtcattcttgaaatttcatgttatagtgacacagtgagtgagtctagttcttacagttatggtgaaatcccgttcggcataactgacggaccacgatactgttatgtactttacaatggcacagctcttaaatatttaggaacattaccacccagtgtaaaggaaattgctattagtaagtggggccatttttatattaatggttacaatttctttagcacatttcctattgattgtatatcttttaatttaaccactggtgttagtggagctttttggacaattgcttacacatcgtatactgaagcattagtacaagttgaaaacacagctattaaaaatgtgacgtattgtaacagtcacattaataacattaaatgttctcaacttactgctaatttgaataatggattttatcctgttgcttcaagtgaagtaggtttcgttaataagagtgttgtgttattacctagctttttcacatacaccgctgtcaatataaccattgatcttggtatgaagcttagtggttatggtcaacccatagcctcgacactaagtaacatcacactaccaatgcaggataacaatactgatgtgtactgtattcgttctaaccaattctcagtttatgttcattccacttgcaaaagttctttatgggacaatatttttaatcaagactgcacggatgttttagaggctacagctgttataaaaactggtacttgtcctttctcatttgataaattgaacaattacttgacttttaacaagttctgtttgtcgttgagtcctgttggtgctaattgcaagtttgatgttgctgcacgtacaagaaccaatgagcaggttgttagaagtctatatgtaatatatgaagaaggagacaacatagtgggtgtaccgtctgatgatagcggtctgcacgatttgtctgtgctacacctagactcctgtacagattacaatatatatggtagaactggtgttggtattattagacgaactaacagtacgctacttagtggcttatattacacatcactatcaggtgatttgttaggctttaaaaatgttagtgatggtgtcatttattctgtgacgccatgtgatgtaagcgcacaagcggc

SEQ ID NO: 28 - protein, 748 aa
MIVLVTCLLLLCSYHTVLSTTNNECIQVNVTQLAGNENLIRDFLFSNFKEEGSVVVGGYYPTEVWYNCSRTARTTAFQYFNNIHAFYFVMEAMENSTGNARGKPLLFHVHGEPVSVIIYISAYRDDVQQRPLLKHGLVCITKNRHINYEQFTSNQWNSTCTGADRKIPFSVIPTDNGTKIYGLEWNDDFVTAYISGRSYHLNINTNWFNNVTLLYSRSSTATWEYSAAYAYQGVSNFTYYKLNNTNGLKTYELCEDYEHCTGYATNVFAPTSGGYIPDGFSFNNWFLLTNSSTFVSGRFVTNQPLLINCLWPVPSFGVAAQEFCFEGAQFSQCNGVSLNNTVDVIRFNLNFTADVQSGMGATVFSLNTTGGVILEISCYSDTVSESSSYSYGEIPFGITDGPRYCYVLYNGTALKYLGTLPPSVKEIAISKWGHFYINGYNFFSTFPIDCISFNLTTGVSGAFWTIAYTSYTEALVQVENTAIKNVTYCNSHINNIKCSQLTANLNNGFYPVASSEVGFVNKSVVLLPSFFTYTAVNITIDLGMKLSGYGQPIASTLSNITLPMQDNNTDVYCIRSNQFSVYVHSTCKSSLWDNIFNQDCTDVLEATAVIKTGTCPFSFDKLNNYLTFNKFCLSLSPVGANCKFDVAARTRTNEQVVRSLYVIYEEGDNIVGVPSDDSGLHDLSVLHLDSCTDYNIYGRTGVGIIRRTNSTLLSGLYYTSLSGDLLGFKNVSDGVIYSVTPCDVSAQA

SEQ ID NO: 29 - DNA (cDNA), 370 bp; double-stranded, unknown
  CDS 3..368
gtggtaaaccattattatttcatgtgcatggtgagcctgttagtgttattatatatatatcggcttatagggatgatgtgcaacaaaggccccttttaaaacatgggttagtgtgcataactaaaaatcgccatattaactatgaacaattcacctccaaccagtggaattccacatgtacgggtgctgacagaaaaattcctttctctgtcatacccacggacaatggaacaaaaatctatggtcttgagtggaatgatgactttgttacagcttatattagtggtcgttcttatcacttgaacatcaatactaattggtttaacaatgtcacacttttgtattcacgctcaagcattgctacctggga

SEQ ID NO: 30 - protein, 122 aa
GKPLLFHVHGEPVSVIIYISAYRDDVQQRPLLKHGLVCITKNRHINYEQFTSNQWNSTCTGADRKIPFSVIPTDNGTKIYGLEWNDDFVTAYISGRSYHLNINTNWFNNVTLLYSRSSIATW

SEQ ID NO: 31 - DNA (cDNA), 4365 bp; double-stranded, unknown
  CDS 1..4362
atgattgtgctcgtaacttgcctcttgttgttatgctcataccacactgtttcgagtacgtcaaacaatgattgtagacaagttaacgtaacacaattagctggcaatgaaaaccttattagagactttttgtttcaaagttttaaagaagaaggaattgtagttgttggtggttattaccctacagaggtgtggtacaactgctctagaacagcaactaccactgcctatgagtattttaataatatacatgccttttattttgatatggaagctatggaaaatagcactggtaatgcacgtggtaaacctctattatttcatgttcatggtgaacctgttagtatcatcatatatatatcagcttatggggatgatgtgcaacaaaggccacttttagaacatgggttattgtgcattactaaaaatcgcaatattgactataacaccttcaccagcaaccagtgggattccatatgtacgggtaatgacagaaaaattcctttctctgtcatacccagggataatggaacaaaaatctatgggcttgagtggaatgatgaatttgttacagcgtatattagtggtcgttcttataattggaacatcaataataactggtttaacaatgtcacacttttgtattcacgctcaagcactgctacctgggaatacagtgctgcatatgtttaccaaggtgtttctaacttcacttattacaagttaaataacaccaatggtttaaaaacctatgaattttgtgaggattatgaatattgcactggctacgccactaatgtctttgctccaactgtgggaggttacatacctgatggatttagttttaacaattggtttttgcttacaaatagctccacttttgttagtggcagatttgtaacaaaccaaccactattagttaactgcttatggccagtgcccagttttggtgtagcagcacaagaattttgttttgaaggtgcgcagtttagtcagtgtagtggtgtatctttaaataacacagtagatgttattagattcaatcttaatttcaccgcagatgtacaatctggtatgggtgctacagtgttttcgttgaatacaacgggtggtgtcattcttgaagtttcatgttataatgacacagtgagtgagtctagtttttacagttatggtgaaattccgttcggcataactgatggaccacggtactgttatgtactttacaatggcacagctcttaagtatttaggaacattaccacctagtgtaaaggaaattgctattagtaagtggggccatttttatattaatggttacaatttctttagcacatttcctattgattgtatatcttttaacttaaccactggtgatagtggagctttttggacaattgcttacacatcgtacactgaggcattagtacaagttgaaaacacagctattaaaaaggtgacgtattgtaacagtcacattaataacattaagtgttctcaacttactgctaatttgaataatggattttatcctgttgcttcaagtgaggttggtcttgtgaataagagtgttgtgttattacctatctttttcgcacataccgctatcaatataaccattgatcttggtatgaagcgtagcggttatggtcaacccatagcatcaacattaagtaacattacactaccaatgcaggataataacacagatgtgtactgtattcgttctaaccagttttcagtttatgttcattctatttgtaagagttctttatgggacaatatttttaatcaagaatgcacggatgttttagatgccacagctgttataaagactggtacttgtcctttctcatttgataaattgaacaattacttaacttttaacaagttctgtttgtcgttgagtcctgttggcgctaactgcaagtttgatgttgccgcacgtacaagaaccaatgagcaagttgttagaagtctatatgtaatatatgaagaaggagacaacatagttggtgtaccgtctgataatagcggtctgcacgatttgtctgtgctacacctagactcctgtacagagtataatatatatggtagaactggtgttggtattattagacaaactaacagtacgctacttagcggcttatattacacatcactatcaggtgatttgttaggctttaaaaatgttagtgatggtgtcatctattctgtgacgccatgtgatgtaagcgcacaagcggctgttattgatggtgccatagttggagctatgacttccattaacagtgaactgttaggtctaaaacactggacaacaacacctaatttttattactactctatatataattatacaaatgagaggactcgtggcactgcaattgacagtaacgatgttgattgtgaacctatcataacctattctaacataggtgtttgtaaaaatggtgctttggtttttattaacgtcacacattctgatggagacgtgcaaccaattagcactggtactgtcacgatacctacaaactttaccatatctgtgcaagtcgaatacattcaggtttacaccacaccagtatcaatagattgtgcaagatacgtttgcaatggtaaccctagatgtaacaaattgttaacacaatatgtttctgcatgtcaaactattgagcaagcacttgcaatgggtgccagacttgaaaacatggaggttgattccatgttgttcgtttctgaaaatgcccttaaattggcatctgttgaggcgttcaatagtacagaaaatttagaccctatttacaaagaatggcctaacataggtggttcttggttaggaggtttaaaagacatactgccgtcccataatagcaaacgtaagtatcgttctgctatagaagacttgctttttgataaggttgtaacttctggtttaggtacagttgatgaagattataaacgttgtacaggtggttatgacatagccgacttagtgtgtgctcaatattacaatggcatcatggtgttacctggtgtagctaatgatgacaagatgactatgtacacagcatctcttgcaggtggtataacactaggtgcacttggtggtggcgccgttgctataccttttgcagtagcagttcaagctagacttaattatgttgctctacaaactgatgtattgaataaaaaccagcagatcctggctaatgctttcaatcaagctattggtaacattacacaggcatttggcaaggttaatgatgctatacatcaaacatcaaaaggtcttgcaactgttgctaaagcattggcaaaagtgcaagatgttgtcaacacacaagggcaagctttaagccacctaacagtacaattgcaaaataattttcaagccattagtagctctattagtgatatttataacaggcttgacgaactgagtgctgatgcacaagttgataggctgattacaggaagacttacagcacttaatgcatttgtgtctcagactctaaccagacaagcggaggttagggctagtagacaacttgccaaggacaaggttaatgaatgtgttagatcccaatctcagagatttggattctgtggtaatggtacacacttgttttcacttgcaaatgcagcaccaaatggcatgattttctttcatacagtgctattaccaacggcttatgaaactgtaacagcttggccaggtatttgtgcttcagatggcgatcgcacttttggacttgtcgttaaagatgtacagttgacgttgtttcgtaacctagatgacaagttctatttgactcccagaactatgtatcagcctagagctgcaactagttctgattttgttcaaattgaggggtgcgatgtgttgtttgtcaatgcaactgtaattgacttgcctagtattatacctgactatattgacatcaatcagactgttcaagatatattagaaaattacagaccaaactggactgtacctgaattgacacttgatatttttaacgcaacctatttaaatctgactggtgaaattgatgacttagaatttaggtcagaaaagctacacaataccactgtagaacttgccattctcattgacaacattaacaacacattagtcaatcttgaatggctcaatagaattgaaacttatgtaaaatggccttggtatgtgtggctactaataggcttagtagtaatattttgcataccattattgctattttgctgttgtagtacaggttgttgtggatgcataggttgcttaggaagttgttgtcactctatgtgtagtagaagacaatttgaaaattatgaaccaattgaaaaagtgcatgtccactaa

SEQ ID NO: 32 - protein, 1454 aa
MIVLVTCLLLLCSYHTVSSTSNNDCRQVNVTQLAGNENLIRDFLFQSFKEEGIVVVGGYYPTEVWYNCSRTATTTAYEYFNNIHAFYFDMEAMENSTGNARGKPLLFHVHGEPVSIIIYISAYGDDVQQRPLLEHGLLCITKNRNIDYNTFTSNQWDSICTGNDRKIPFSVIPRDNGTKIYGLEWNDEFVTAYISGRSYNWNINNNWFNNVTLLYSRSSTATWEYSAAYVYQGVSNFTYYKLNNTNGLKTYEFCEDYEYCTGYATNVFAPTVGGYIPDGFSFNNWFLLTNSSTFVSGRFVTNQPLLVNCLWPVPSFGVAAQEFCFEGAQFSQCSGVSLNNTVDVIRFNLNFTADVQSGMGATVFSLNTTGGVILEVSCYNDTVSESSFYSYGEIPFGITDGPRYCYVLYNGTALKYLGTLPPSVKEIAISKWGHFYINGYNFFSTFPIDCISFNLTTGDSGAFWTIAYTSYTEALVQVENTAIKKVTYCNSHINNIKCSQLTANLNNGFYPVASSEVGLVNKSVVLLPIFFAHTAINITIDLGMKRSGYGQPIASTLSNITLPMQDNNTDVYCIRSNQFSVYVHSICKSSLWDNIFNQECTDVLDATAVIKTGTCPFSFDKLNNYLTFNKFCLSLSPVGANCKFDVAARTRTNEQVVRSLYVIYEEGDNIVGVPSDNSGLHDLSVLHLDSCTEYNIYGRTGVGIIRQTNSTLLSGLYYTSLSGDLLGFKNVSDGVIYSVTPCDVSAQAAVIDGAIVGAMTSINSELLGLKHWTTTPNFYYYSIYNYTNERTRGTAIDSNDVDCEPIITYSNIGVCKNGALVFINVTHSDGDVQPISTGTVTIPTNFTISVQVEYIQVYTTPVSIDCARYVCNGNPRCNKLLTQYVSACQTIEQALAMGARLENMEVDSMLFVSENALKLASVEAFNSTENLDPIYKEWPNIGGSWLGGLKDILPSHNSKRKYRSAIEDLLFDKVVTSGLGTVDEDYKRCTGGYDIADLVCAQYYNGIMVLPGVANDDKMTMYTASLAGGITLGALGGGAVAIPFAVAVQARLNYVALQTDVLNKNQQILANAFNQAIGNITQAFGKVNDAIHQTSKGLATVAKALAKVQDVVNTQGQALSHLTVQLQNNFQAISSSISDIYNRLDELSADAQVDRLITGRLTALNAFVSQTLTRQAEVRASRQLAKDKVNECVRSQSQRFGFCGNGTHLFSLANAAPNGMIFFHTVLLPTAYETVTAWPGICASDGDRTFGLVVKDVQLTLFRNLDDKFYLTPRTMYQPRAATSSDFVQIEGCDVLFVNATVIDLPSIIPDYIDINQTVQDILENYRPNWTVPELTLDIFNATYLNLTGEIDDLEFRSEKLHNTTVELAILIDNINNTLVNLEWLNRIETYVKWPWYVWLLIGLVVIFCIPLLLFCCCSTGCCGCIGCLGSCCHSMCSRRQFENYEPIEKVHVH

SEQ ID NO: 33 - DNA (cDNA), 2246 bp; unknown-stranded, unknown
  CDS 1..2244
atgattgtgctcgtaacttgcctcttgttgttatgttcataccacacagttttgagtacaacaaataatgaatgcatacaagttaacgtaacacaattggctggcaatgaaaaccttatcagagattttctgtttagtaactttaaagaagaaggaagtgtagttgttggtggttattaccctacagaggtgtggtacaactgctctagaacagctcgaactactgcctttcagtattttaataatatacatgccttttattttgttatggaagccatggaaaatagcactggtaatgcacgtggtaaaccattattatttcatgtgcatggtgagcctgttagtgttattatatatatatcggcttatagggatgatgtgcaacaaaggccccttttaaaacatgggttagtgtgcataactaaaaatcgccatattaactatgaacaattcacctccaaccagtggaattccacatgtacgggtgctgacagaaaaattcctttctctgtcatacccacggacaatggaacaaaaatctatggtcttgagtggaatgatgactttgttacagcttatattagtggtcgttcttatcacttgaacatcaatactaattggtttaacaatgtcacacttttgtattcacgctcaagcactgctacctgggaatacagtgctgcatatgcttaccaaggtgtttctaacttcacttattacaagttaaataacaccaatggtctaaaaacctatgaattatgtgaagattatgaacattgcactggctatgctaccaatgtatttgctccgacatcaggtggttacatacctgatggatttagttttaayaattggttcttgcttacaaatagttccacttttgttagtggcaggtttgtaacaaatcaaccattattgattaattgcttgtggccagtgcccagttttggtgtagcagcacaagaattttgttttgaaggtgcacagtttagccaatgtaatggtgtgtctttaaataacacagtggatgttattagattcaaccttaatttcactgcagatgtacaatctggtatgggtgctacagtattttcactgaatacaacaggtggtgtcattcttgaaatttcatgttatagtgacacagtgagtgagtctagttcttacagttatggtgaaatcccgttcggcataactgacggaccacgatactgttatgtactttacaatggcacagctcttaaatatttaggaacattaccacccagtgtaaaggaaattgctattagtaagtggggccatttttatattaatggttacaatttctttagcacatttcctattgrttgtatatcttttaatttaaccactggtgttagtggagctttttggacaattgcttacacatcgtatactgaagcattagtacaagttgaaaacacagctattaaaaatgtgacgtattgtaacagtcacattaataacattaaatgttctcaacttactgctaatttgaataatggattttatcctgttgcttcaagtgaagtaggtttcgttaataagagtgttgtgttattacctagctttttcacatacaccgctgtcaatataaccattgatcttggtatgaagcttagtggttatggtcaacccatagcctcgacactaagtaacatcacactaccaatgcaggataacaatactgatgtgtactgtattcgttctaaccaattctcagtttatgttcattccacttgcaaaagttctttatgggacaatatttttaatcaagactgcacggatgttttagaggctacagctgttataaaaactggtacttgtcctttctcatttgataaattgaacaattacttgacttttaacaagttctgtttgtcgttgagtcctgttggtgctaattgcaagtttgatgttgctgcacgtacaagaaccaatgagcaggttgttagaagtctatatgtaatatatgaagaaggagacaacatagtgggtgtaccgtctgatratagcggtctgcacgatttgtctgtgctacacctagactcctgtacagattacaatatatatggtagaactggtgttggtattattagacgaactaacagtacgctacttagtggcttatattacacatcactatcaggtgatttgttaggctttaaaaatgttagtgatggtgtcatttattctgtgacgccatgtgatgtaagcgcacaagcggc

SEQ ID NO: 34 - protein, 748 aa
MIVLVTCLLLLCSYHTVLSTTNNECIQVNVTQLAGNENLIRDFLFSNFKEEGSVVVGGYYPTEVWYNCSRTARTTAFQYFNNIHAFYFVMEAMENSTGNARGKPLLFHVHGEPVSVIIYISAYRDDVQQRPLLKHGLVCITKNRHINYEQFTSNQWNSTCTGADRKIPFSVIPTDNGTKIYGLEWNDDFVTAYISGRSYHLNINTNWFNNVTLLYSRSSTATWEYSAAYAYQGVSNFTYYKLNNTNGLKTYELCEDYEHCTGYATNVFAPTSGGYIPDGFSFNNWFLLTNSSTFVSGRFVTNQPLLINCLWPVPSFGVAAQEFCFEGAQFSQCNGVSLNNTVDVIRFNLNFTADVQSGMGATVFSLNTTGGVILEISCYSDTVSESSSYSYGEIPFGITDGPRYCYVLYNGTALKYLGTLPPSVKEIAISKWGHFYINGYNFFSTFPIXCISFNLTTGVSGAFWTIAYTSYTEALVQVENTAIKNVTYCNSHINNIKCSQLTANLNNGFYPVASSEVGFVNKSVVLLPSFFTYTAVNITIDLGMKLSGYGQPIASTLSNITLPMQDNNTDVYCIRSNQFSVYVHSTCKSSLWDNIFNQDCTDVLEATAVIKTGTCPFSFDKLNNYLTFNKFCLSLSPVGANCKFDVAARTRTNEQVVRSLYVIYEEGDNIVGVPSDXSGLHDLSVLHLDSCTDYNIYGRTGVGIIRRTNSTLLSGLYYTSLSGDLLGFKNVSDGVIYSVTPCDVSAQA

SEQ ID NO: 35 - DNA (cDNA), 27 bp; double-stranded, unknown
  CDS 1..27
tcgagtacgtcaaacaatgattgtaga

SEQ ID NO: 36 - protein, 9 aa
SSTSNNDCR

SEQ ID NO: 37 - DNA (cDNA), 24 bp; double-stranded, unknown
  CDS 1..24
caaagttttaaagaagaaggaatt

SEQ ID NO: 38 - protein, 8 aa
QSFKEEGI

SEQ ID NO: 39 - DNA (cDNA), 18 bp; double-stranded, unknown
  CDS 1..18
gcaactaccactgcctat

SEQ ID NO: 40 - protein, 6 aa
ATTTAY

SEQ ID NO: 41 - DNA (cDNA), 150 bp; double-stranded, unknown
  CDS 1..150
ggggatgatgtgcaacaaaggccacttttagaacatgggttattgtgcattactaaaaatcgcaatattgactataacaccttcaccagcaaccagtgggattccatatgtacgggtaatgacagaaaaattcctttctctgtcataccc

SEQ ID NO: 42 - protein, 50 aa
GDDVQQRPLLEHGLLCITKNRNIDYNTFTSNQWDSICTGNDRKIPFSVIP

SEQ ID NO: 43 - DNA (cDNA), 18 bp; double-stranded, unknown
  CDS 1..18
aatattgactataacacc

SEQ ID NO: 44 - protein, 6 aa
NIDYNT

SEQ ID NO: 45 - DNA (cDNA), 66 bp; double-stranded, unknown
  CDS 1..66
ttgtgcattactaaaaatcgcaatattgactataacaccttcaccagcaaccagtgggattccata

SEQ ID NO: 46 - protein, 22 aa
LCITKNRNIDYNTFTSNQWDSI

SEQ ID NO: 47 - DNA (cDNA), 24 bp; double-stranded, unknown
  CDS 1..24
aatcgcaatattgactataacacc

SEQ ID NO: 48 - protein, 8 aa
NRNIDYNT

SEQ ID NO: 49 - DNA (cDNA), 18 bp; double-stranded, unknown
  CDS 1..18
aattggaacatcaataat

SEQ ID NO: 50 - protein, 6 aa
NWNINN

SEQ ID NO: 51 - DNA (cDNA), 24 bp; unknown-stranded, unknown
  CDS 1..24
atctttttcgcacataccgctatc

SEQ ID NO: 52 - protein, 8 aa
IFFAHTAI

SEQ ID NO: 53 - DNA (cDNA), 377 bp; double-stranded, unknown
  CDS 1..375
aatgctcgtggtaaaccattattatttcatgtgcatggtgagcctgttagtgttattatatatatatcggcttatagggatgatgtgcaacaaaggccccttttaaaacatgggttagtgtgcataactaaaaatcgccatattaactatgaacaattcacctccaaccagtggaattccacatgtacgggtgctgacagaaaaattcctttctctgtcatacccacggacaatggaacaaaaatctatggtcttgagtggaatgatgactttgttacagcttatattagtggtcgttcttatcacttgaacatcaatactaattggtttaacaatgtcacacttttgtattcacgctcaagcactgctacctggga

SEQ ID NO: 54 - protein, 125 aa
NARGKPLLFHVHGEPVSVIIYISAYRDDVQQRPLLKHGLVCITKNRHINYEQFTSNQWNSTCTGADRKIPFSVIPTDNGTKIYGLEWNDDFVTAYISGRSYHLNINTNWFNNVTLLYSRSSTATW